(19) United States
(12) Patent Application Publication
(10) Pub. No.: US 2011/0006287 A1
(43) Pub. Date: Jan. 13, 2011

(54) POLYMERS WITH TUNABLE BAND GAPS FOR PHOTONIC AND ELECTRONIC APPLICATIONS

(76) Inventors: Wei You, Durham, NC (US); Shengqiang Xiao, Wuhan, NC (US)

Correspondence Address: MYERS BIGEL SIBLEY & SAJOVEC, PO BOX 37428, RALEIGH, NC, 27627, US

(21) Appl. No.: 12/726,896

(22) Filed: Mar. 18, 2010

Related U.S. Application Data
(60) Provisional application No. 61/224,583, filed on Jul. 10, 2009.

(51) Int. Cl. H01L 51/30; C08G 75/00; C07D 513/04; C07D 495/14; C07D 513/14; C07D 495/22

(52) U.S. Cl. 257/40; 528/380; 528/377; 528/321; 548/151; 548/149; 544/343; 548/423; 548/418; 548/148; 544/342; 549/41; 257/E51.027

(57) ABSTRACT

A copolymer comprising at least one donor monomer and at least one acceptor monomer is described. The polymer may optionally further comprise, consist or consist essentially of at least one additional comonomer. Various donor monomers, acceptor monomers and additional comonomers are also described. The polymer is useful in the manufacture of microelectronic devices such as optoelectronic devices.

### RELATED APPLICATIONS

This application claims the benefit of commonly owned, copending application Ser. No. 61/224,583, filed Jul. 10, 2009, the disclosure of which is incorporated by reference herein in its entirety.

### GOVERNMENT FUNDING

This invention was made with Government Support from the National Science Foundation STC Program at UNC Chapel Hill (CHE-9876674). The US Government has certain rights to this invention.

### FIELD OF THE INVENTION

The present invention concerns monomers, polymers, and semiconductor devices comprising such polymers.

### BACKGROUND OF THE INVENTION

Tremendous research efforts have been devoted to the development of polymer-based organic photovoltaic (OPV) cells during the last two decades due to projected advantages of these solar cells over their inorganic counterparts, including flexibility, facile processing and manipulation, low weight and low cost. The mechanism by which light is converted into electricity in these OPV devices consists of the following fundamental steps: light absorption, exciton generation, exciton migration, exciton dissociation and charge transport. The bulk heterojunction (BHJ) of regioregular poly(3-hexylthiophene) (RR-P3HT) and [6,6]-phenyl C₆₁-butyric acid methyl ester (PCBM) represents one of the most successful systems with reproducible efficiencies approaching 5% after careful optimization.^{1,2} To further improve the performance of polymer-based BHJs, one has to carefully address the following issues. First, the HOMO and LUMO energy levels of the donor and acceptor components need to have optimal offset to maximize the attainable open circuit voltage (V_{oc}). Secondly, the active layer should have compatible absorption with respect to the solar spectrum to maximize the efficiencies of exciton generation, which sets the upper limit for the short circuit current J_{sc}. Finally, the morphology of the active layer, which governs the physical interaction between the donor and the acceptor, should be optimized to promote charge separation and favorable transport of photogenerated charges and to maximize the attainable J_{sc} and fill factor (FF).^{3,4}

Fulfilling these requirements presents serious challenges in the design of new semiconductive conjugated polymers to be employed as active donors in polymer-based BHJ photovoltaic devices. For example, a number of low band gap polymers have been developed in recent years in the attempt to increase the device efficiency by improving light harvesting.⁵⁻¹⁵ However, none of them can outperform P3HT in terms of energy conversion efficiency, mainly due to high lying HOMO energy level with regard to the LUMO of the acceptor (usually PCBM), which reduces the V_{oc}, or ill-defined morphology of the active blend, which reduces the J_{sc} and FF (or both). In our search for new donor materials, polycyclic aromatic moieties drew our attention. Their rigidly enforced planarity would benefit more effective π electron delocalization when incorporated into the conjugated polymer backbone, which would lead to decreased optical band gaps while providing π interactions between polymer chains in thin solid films, thereby improving charge carrier mobility in devices.¹⁶⁻²¹

### SUMMARY OF THE INVENTION

A first aspect of the present invention is a copolymer comprising, consisting of or consisting essentially of at least one (e.g., 1, 2, 3, 4) donor monomer and at least one (e.g., 1, 2, 3, 4) acceptor monomer. The polymer may optionally further comprise, consist or consist essentially of at least one (e.g., 1, 2, 3, 4) additional comonomer. Various donor monomers, acceptor monomers and additional comonomers are described below.

A further aspect of the invention is a polymer comprising, consisting of or consisting essentially of at least one (e.g., 1, 2, 3, 4) donor monomer selected from a subset of the donor monomers described below. Such polymers may be homopolymers or copolymers.

A further aspect of the invention is a donor monomer as described herein.

A further aspect of the present invention is the use of a polymer as described herein as a charge-transport, semiconducting, el conducting, photoconducting, or light emitting material.

A further aspect of the present invention is a microelectronic device comprising a polymer as described herein (e.g. as said heterojunction therein).

The present invention is explained in greater detail in the drawings herein and the specification set forth below. The disclosures of all United States patent references cited herein are incorporated by reference herein in their entirety.

### BRIEF DESCRIPTION OF THE DRAWINGS

FIG. 1. The structure of three alternating copolymers PBDT, PNDT, PQDT.

FIG. 2. Normalized UV-Vis absorption spectra of PBDT, PNDT and PQDT in (a) toluene solution and (b) as thin films.

FIG. 3. Energy diagram with HOMO/LUMO levels of PBDT, PNDT, PQDT and PCBM in relation to the work functions of the electrode materials ITO/PEDOT:PSS and Al in a BHJ polymer/PCBM OPV device.

FIG. 4. Typical I-V characteristics (AM 1.5G, 100 mW/cm²) of ITO/PEDOT:PSS(45 nm)/copolymer:PCBM (1:1.6, w/w)/Al (100 nm) devices.

FIG. 5. IPCE spectra (gray circled line) of BHJ photovoltaic devices ITO/PEDOT:PSS (45 nm)/copolymer:PCBM (1:1.6, w/w)/Al (100 nm) and the optical absorptions for the corresponding films of the blend from polymers and PCBM (black solid line) with the thickness of 100 nm.

### DETAILED DESCRIPTION OF THE PREFERRED EMBODIMENTS

“Alkyl” as used herein alone or as part of another group, refers to a straight or branched chain hydrocarbon containing from 1 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like.

“Fluoroalkyl” as used herein refers to an alkyl group as described above, substituted one or more times (e.g., 1, 2, 3, 4, 6, 8, etc.) with a fluoro group. When all hydrogens of an alkyl group are replaced with fluorine

“Alkoxy” as used herein alone or as part of another group, refers to an alkyl or loweralkyl group, as defined herein (and thus including substituted versions such as fluorolyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and loweralkyl above.

“Reactive functional group” as used herein includes any suitable reactive group. Examples include, but are not limited to, reactive halide functional groups (e.g., fluoro, chloro, bromo, iodo), reactive boron functional groups (e.g., boronic acids, boronic esters, boranes), and reactive tin functional groups (e.g., trialkyl tin). Such reactive functional groups are known. See, e.g., U.S. Pat. Nos. 7,534,503; 7,348,428; and 5,777,070

As noted above, the present invention provides a copolymer comprising, consisting of or consisting essentially of at least one (e.g., 1, 2, 3, 4) donor monomer and at least one (e.g., 1, 2, 3, 4) acceptor monomer. The donor monomer can be selected from the group consisting of:

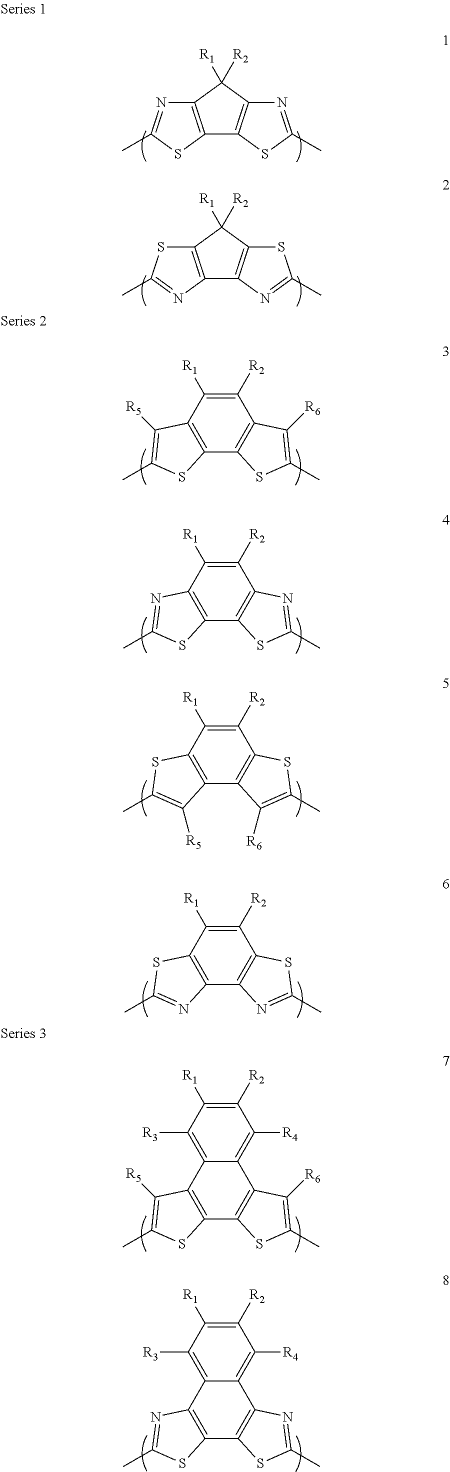

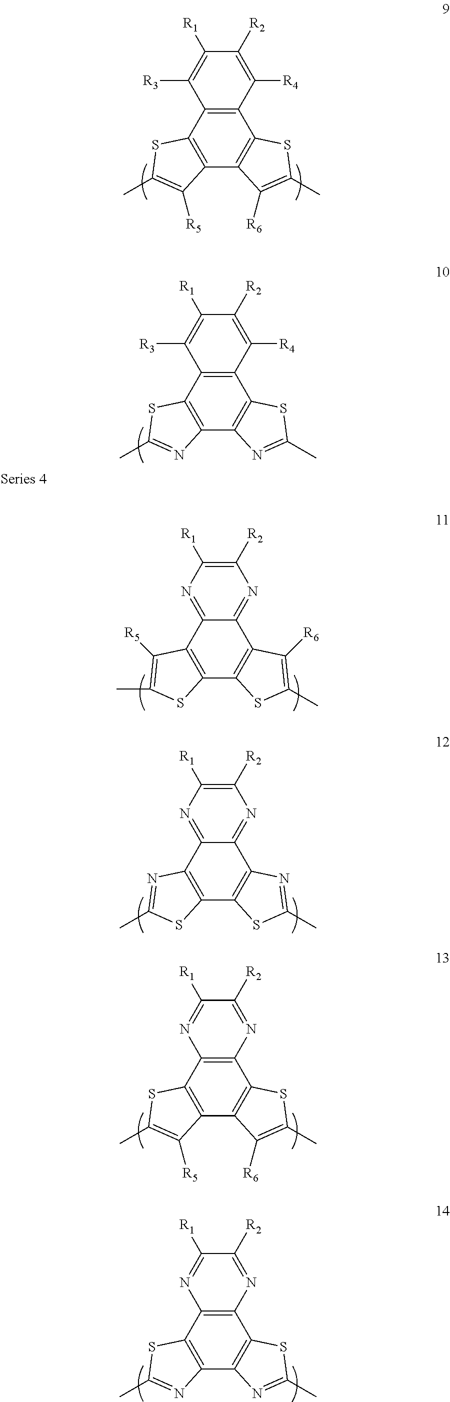

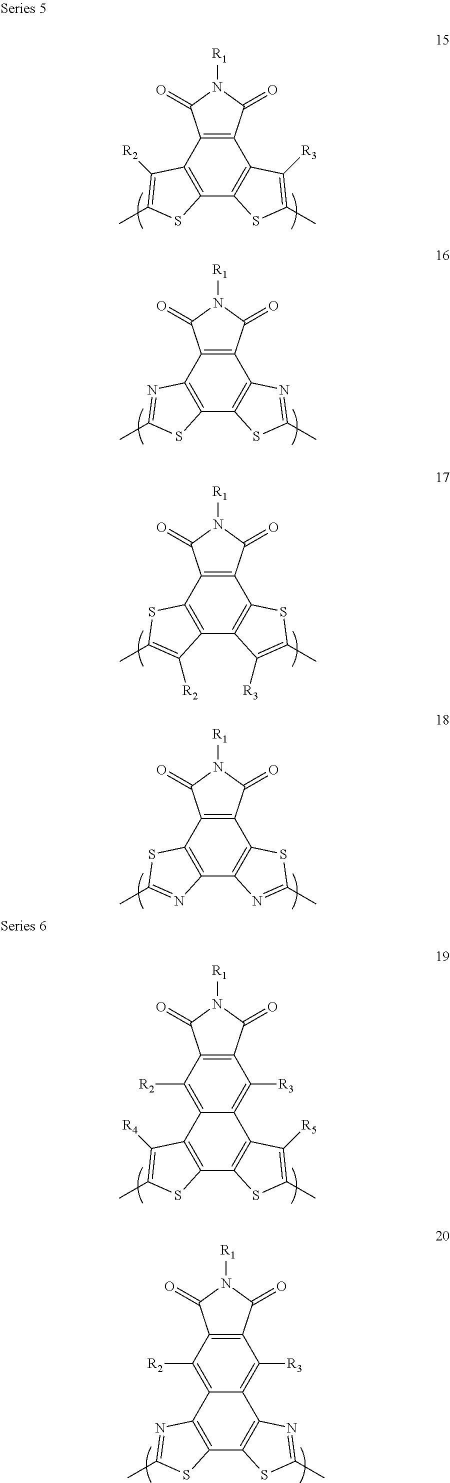

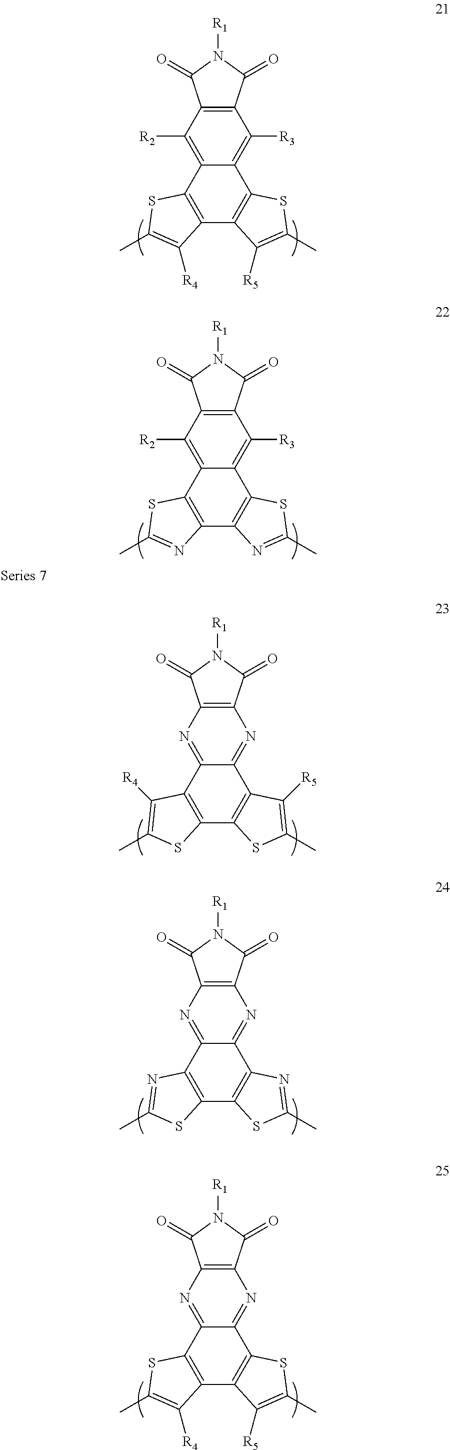

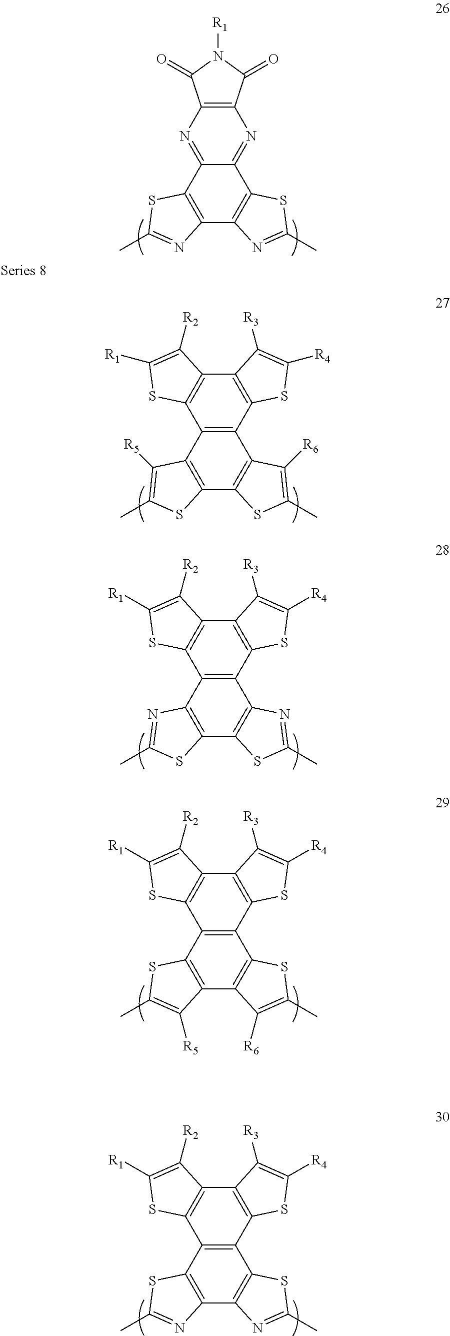

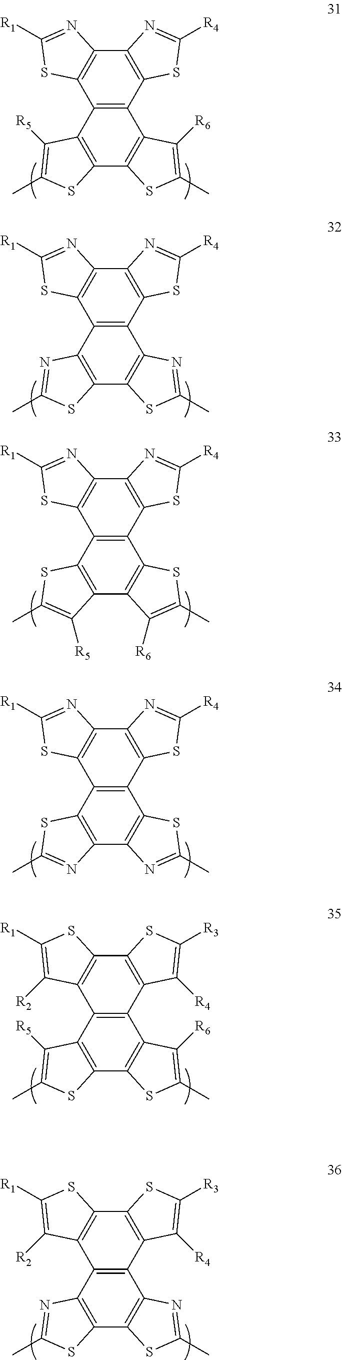

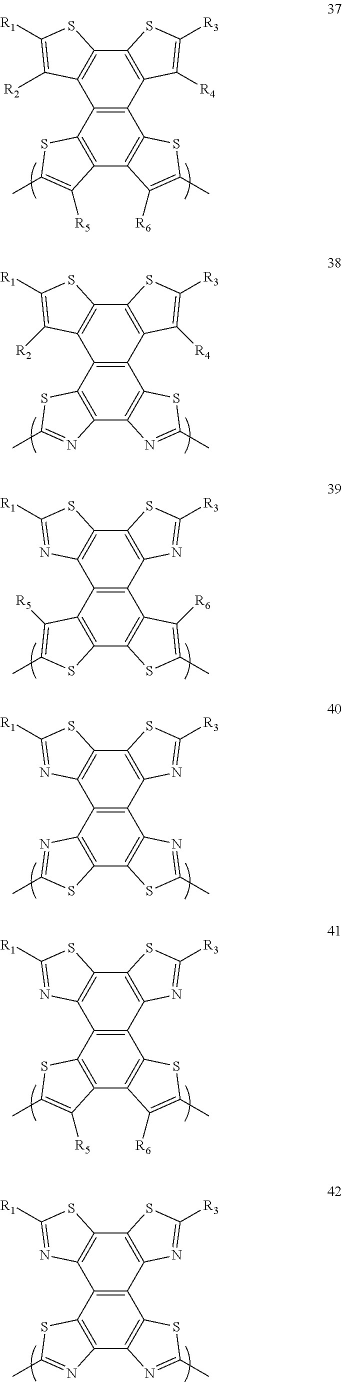

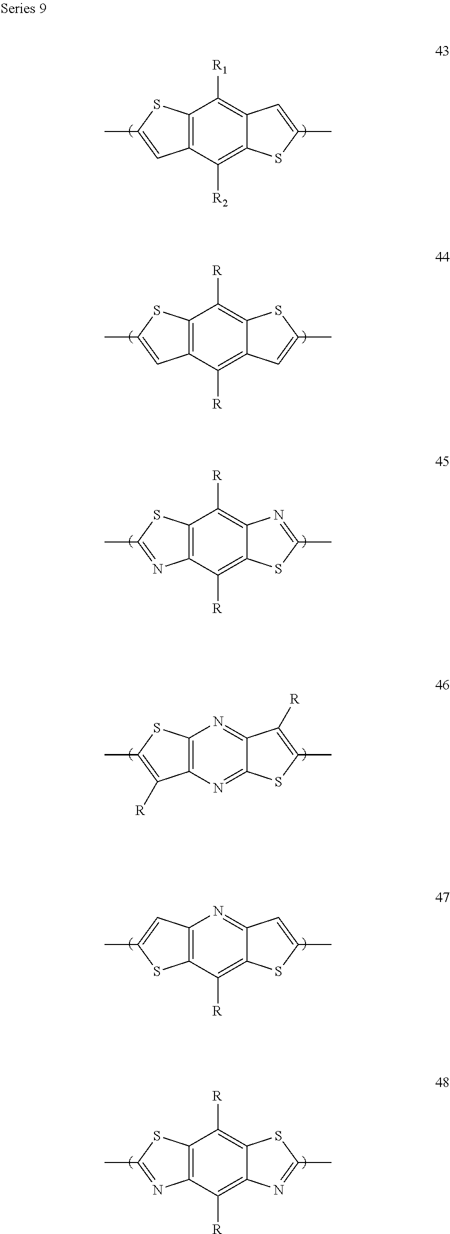

and the acceptor monomer can be selected from the group consisting of:

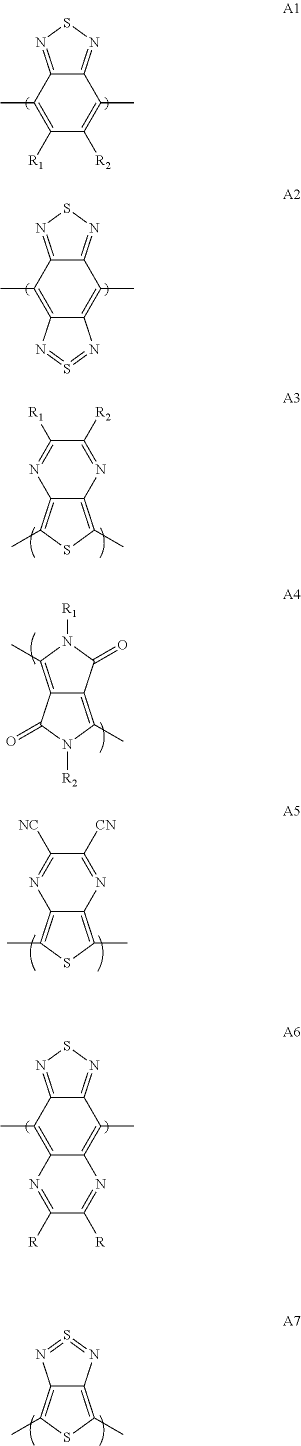

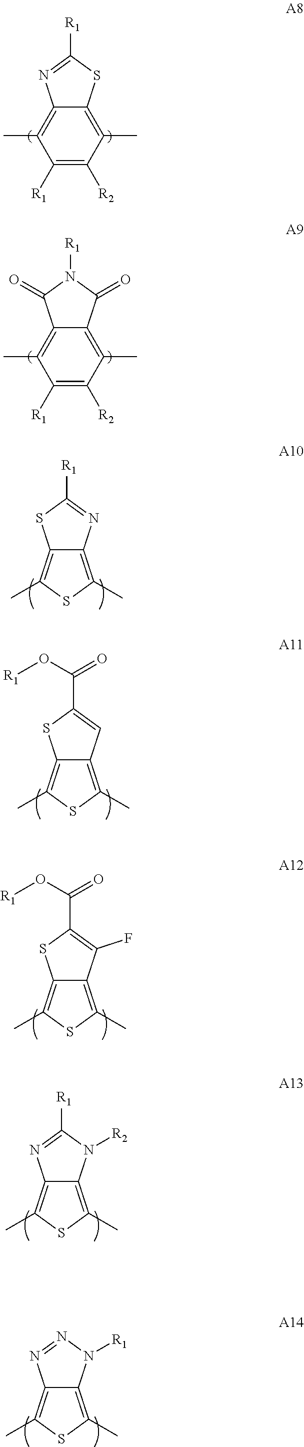

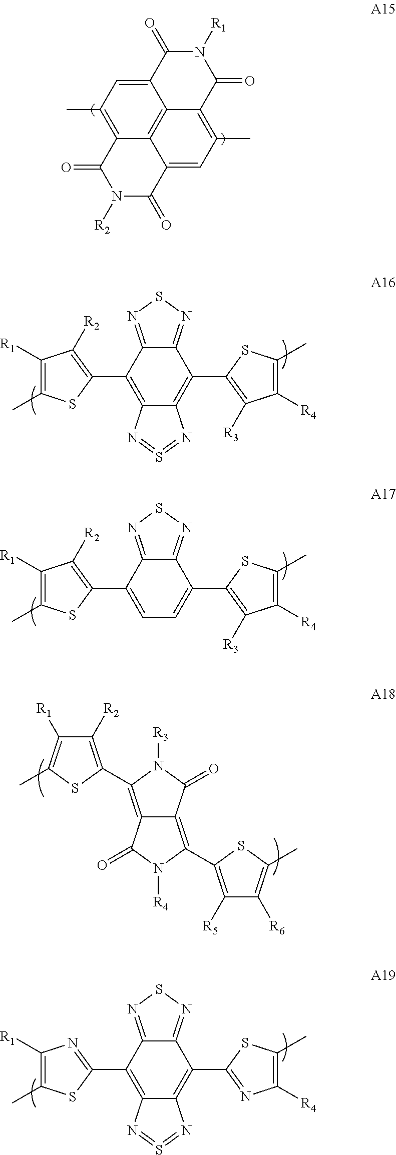

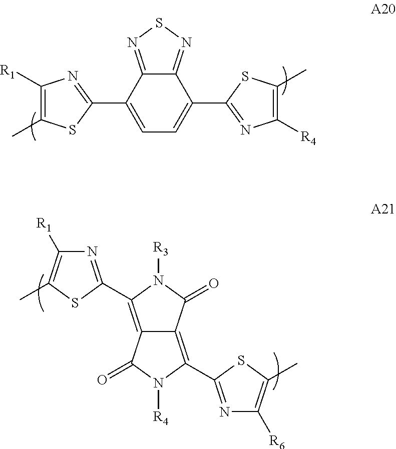

wherein each R₁, R₂, R₃, R₄, R₅, and R₆ is independently selected from the group consisting of H, C1-C20 alkyl, C1-C20 fluoroalkyl, C1-C20 alkoxy, C1-C20 fluoroalkoxy, halo, and aryl. Some embodiments are subject to the proviso that said acceptor monomer is not A1, A3, A11 or A12 when said donor monomer is 43. More generally, some embodiments are subject to the proviso that said acceptor monomer is not A1, A2, A3, A10, A11, A12, A13 or A14 when said donor monomer is 43, 44, 46 or 47. The polymer may optionally further comprise, consist or consist essentially of at least one (e.g., 1, 2, 3, 4) additional comonomer, such as a:

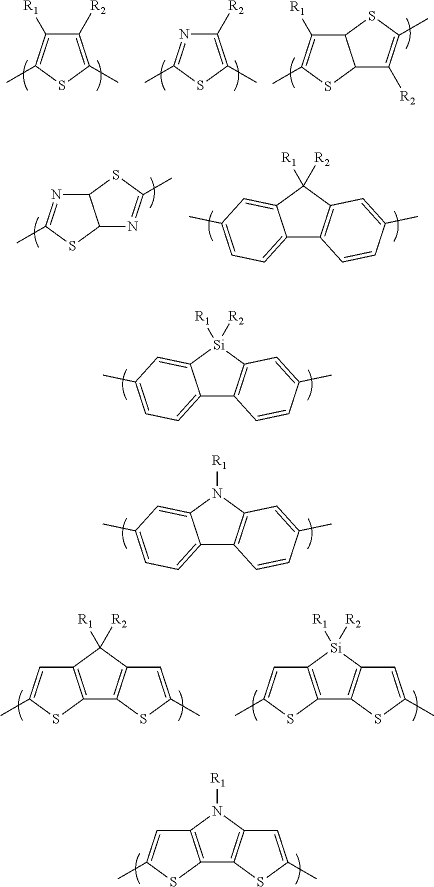

or combinations thereof, wherein R₁ and R₂ are as given above. In some embodiments, the polymer has the formula:

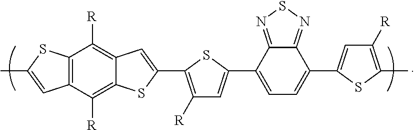

wherein each R is independently as given above. In some embodiments, the polymer has the formula:

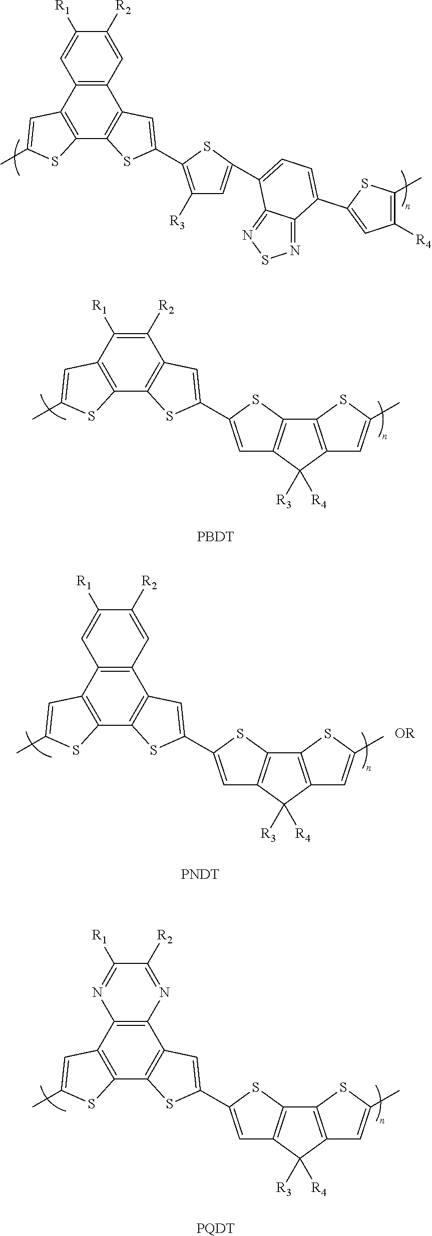

wherein each R₁, R₂, R₃ and R₄ is independently as given above.

A further aspect of the invention is a polymer comprising, consisting of or consisting essentially of at least one (e.g., 1, 2, 3, 4) donor monomer selected from the group consisting of:

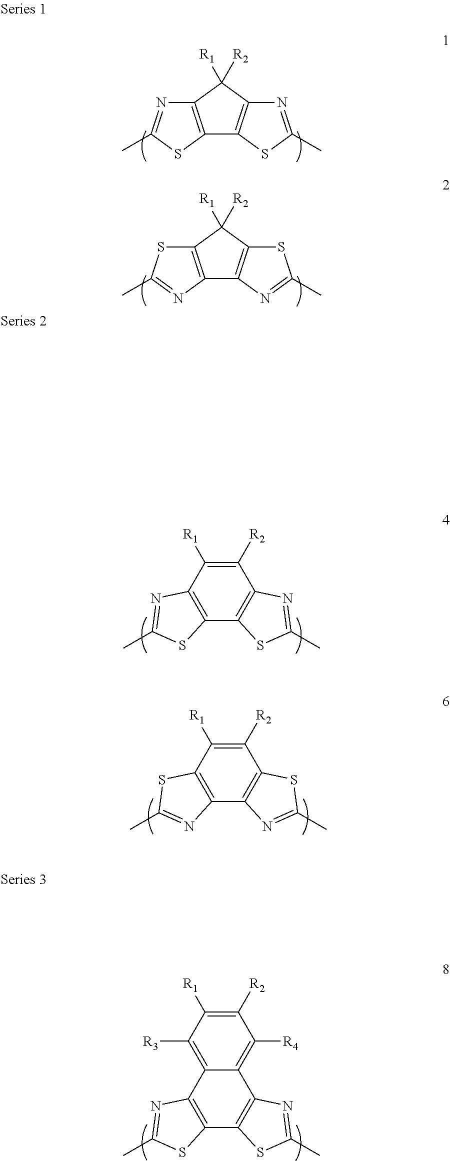

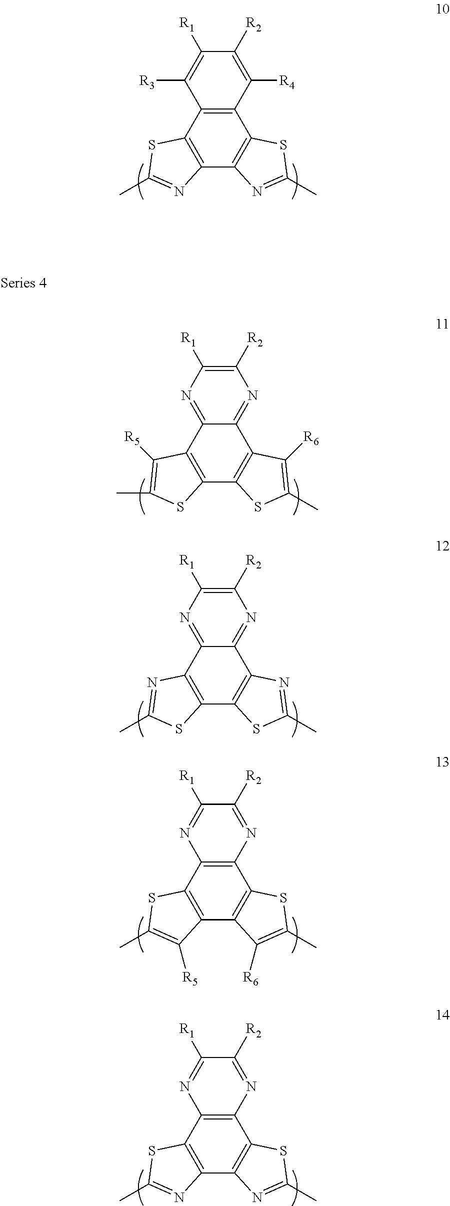

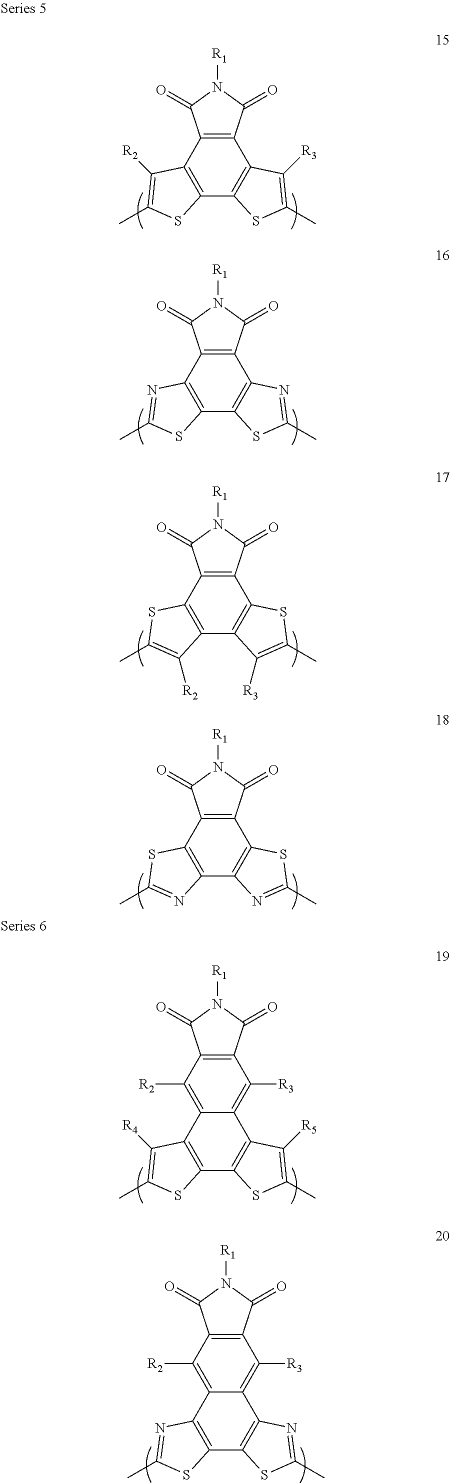

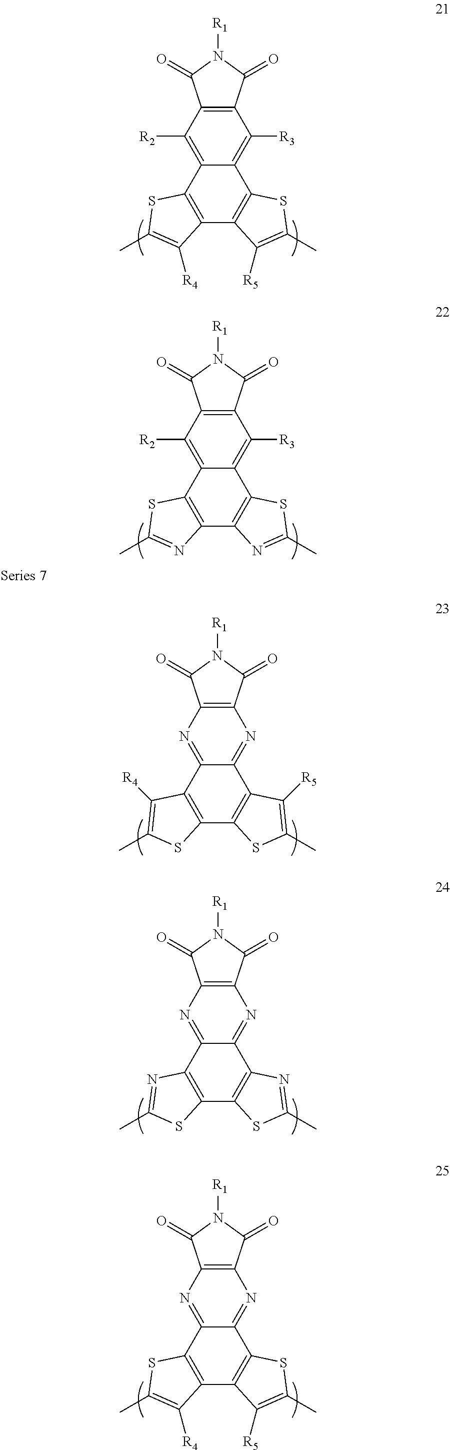

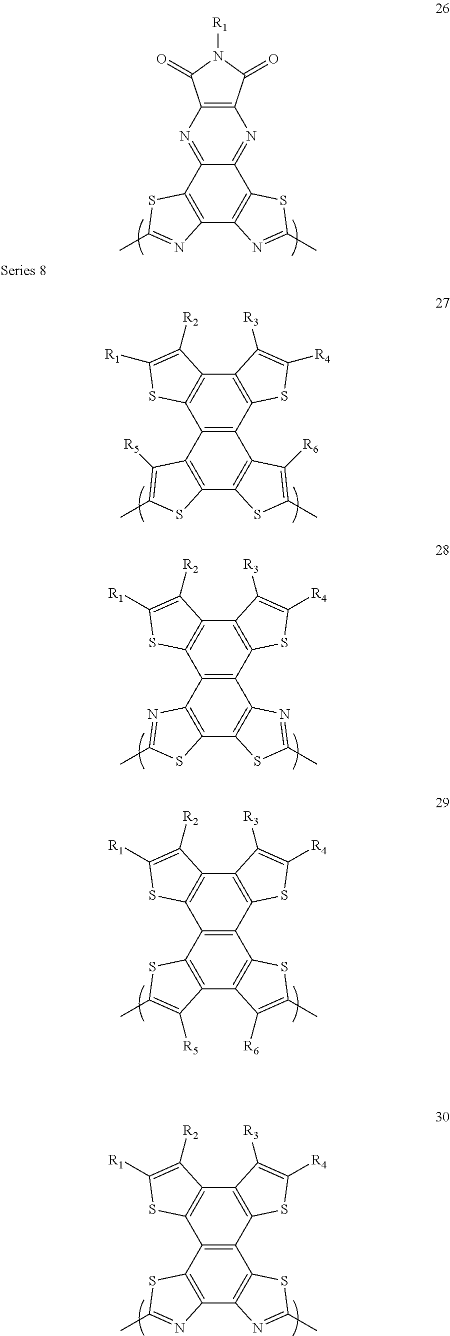

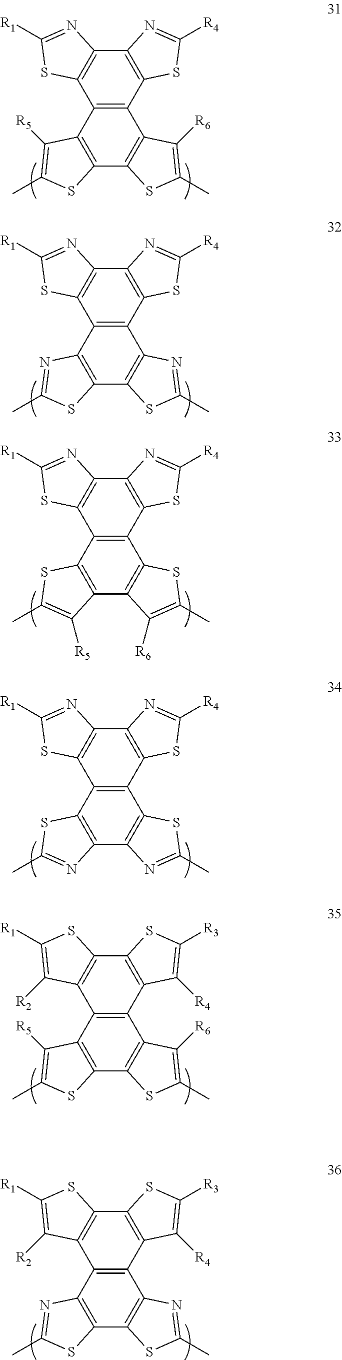

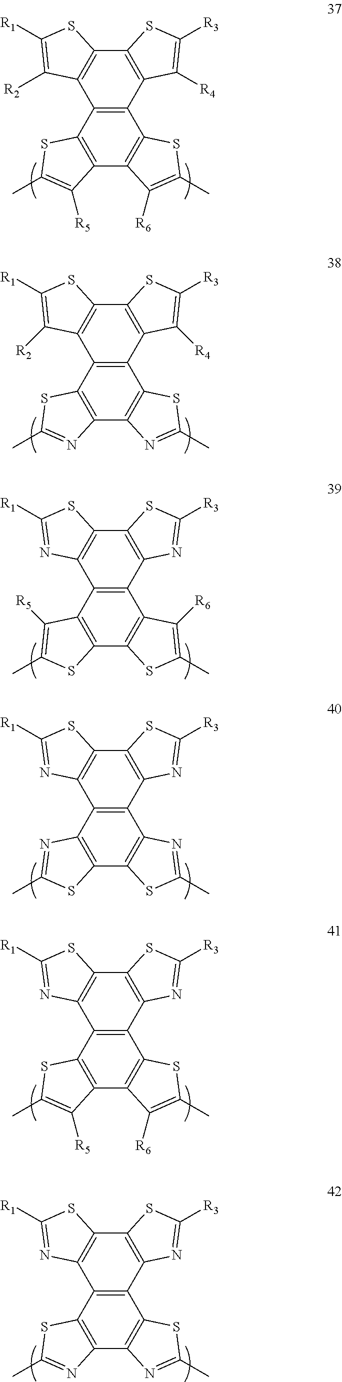

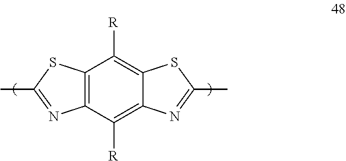

wherein each R₁, R₂, R₃, R₄, R₅, and R₆ is as given above. Such polymers may be homopolymers or copolymers with at least one (e.g., 1, 2, 3, 4) additional monomer. When a copolymer, the copolymer may comprise an acceptor monomer such as described above, or an additional comonomer such as described above.

In some embodiments, the polymers described above have a number average molecular weight of from 500 to 1,000,000 grams per mole.

Monomers illustrated above are shown with open bonds. It will be understood that open bonds can be replaced with a suitable substituent (e.g., X and Y respectively) when the monomer is expressed as a compound per se. For example, the monomers may be expressed as:

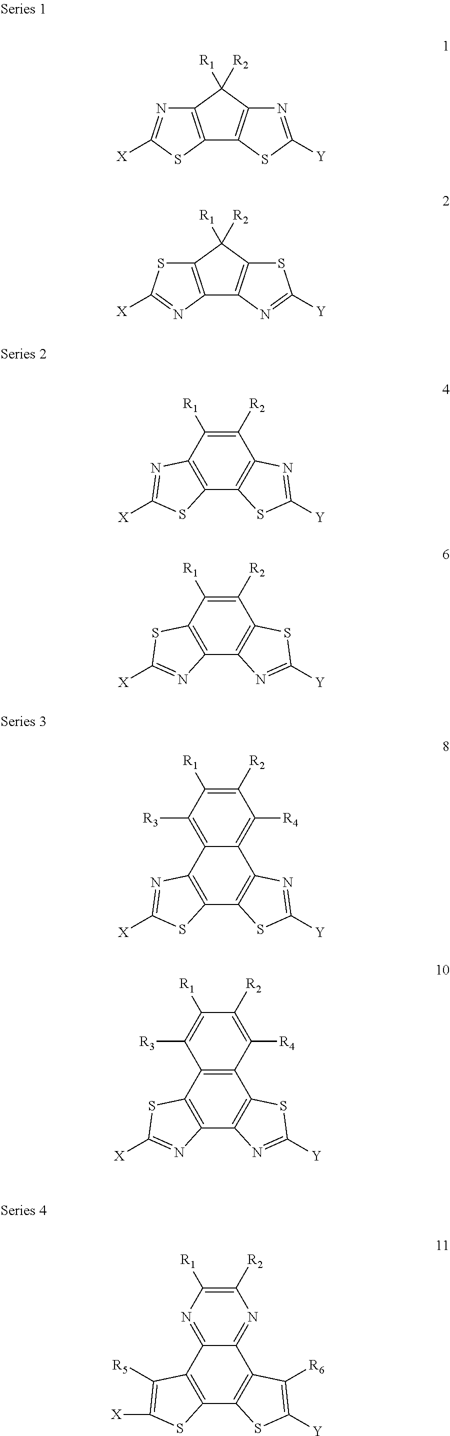

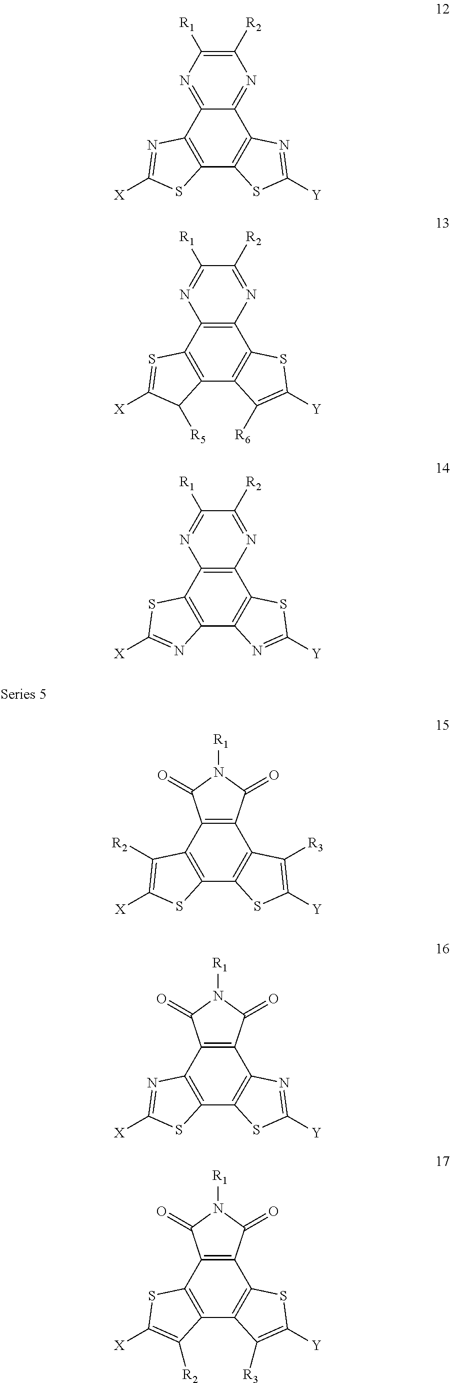

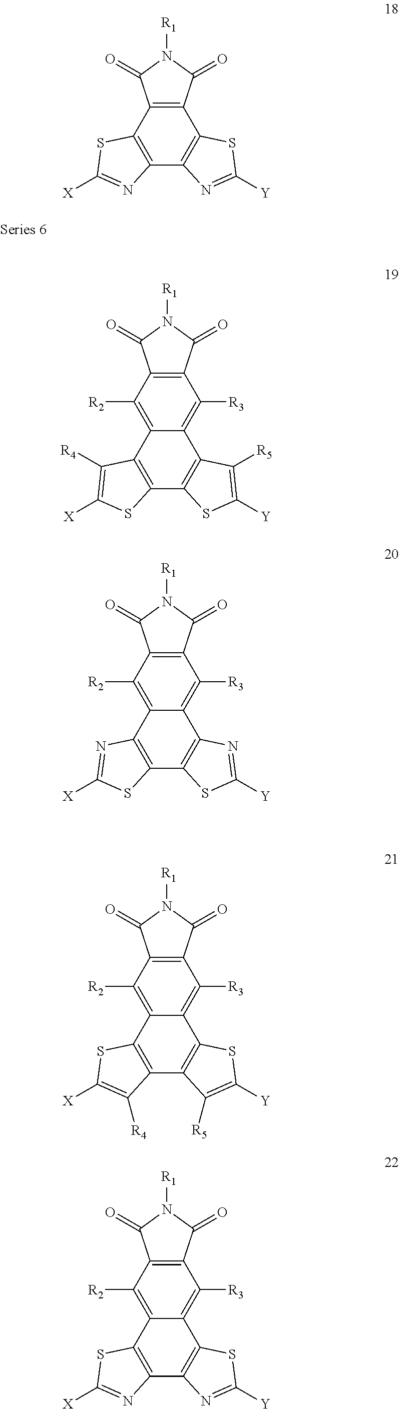

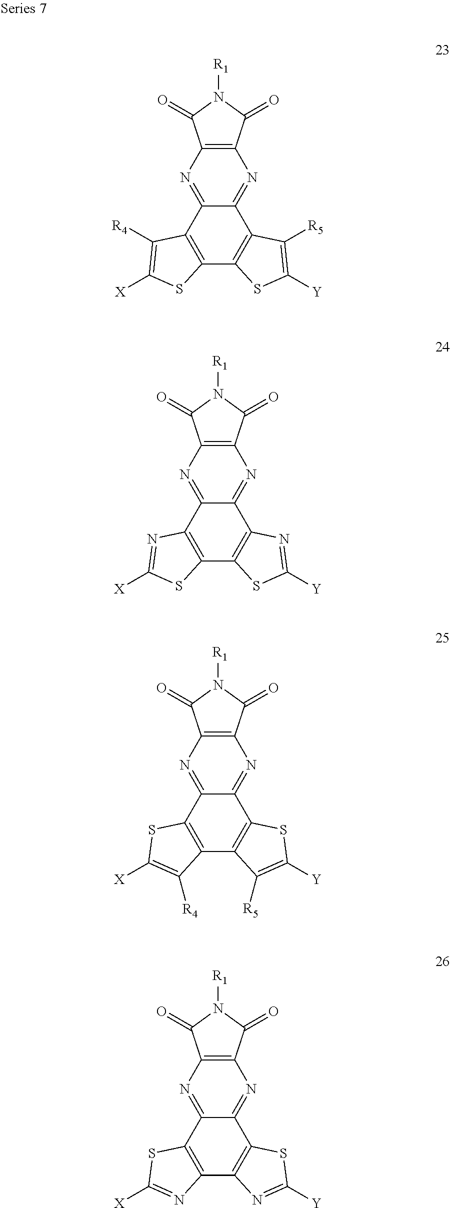

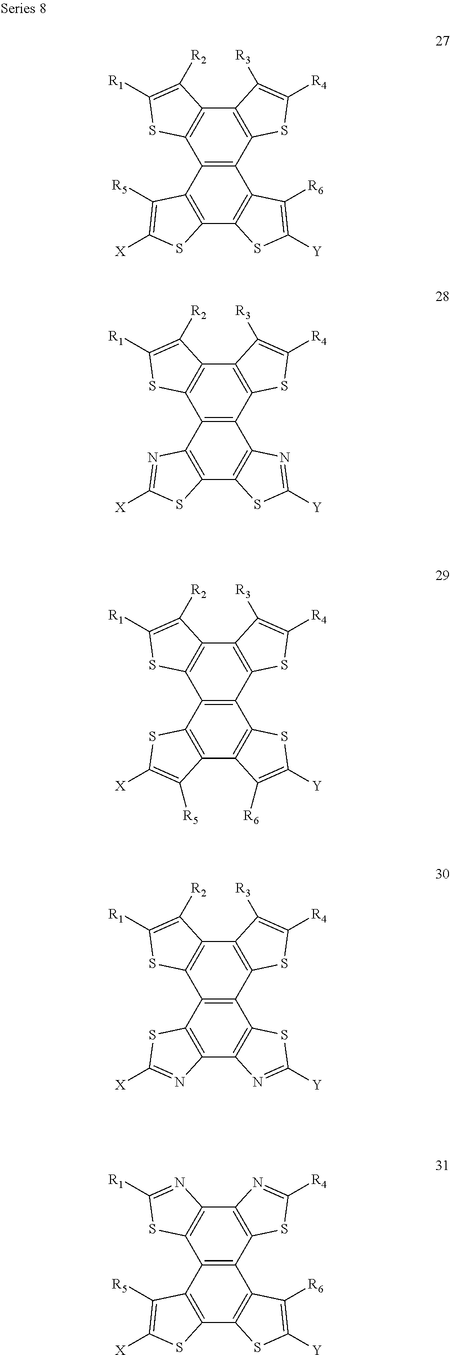

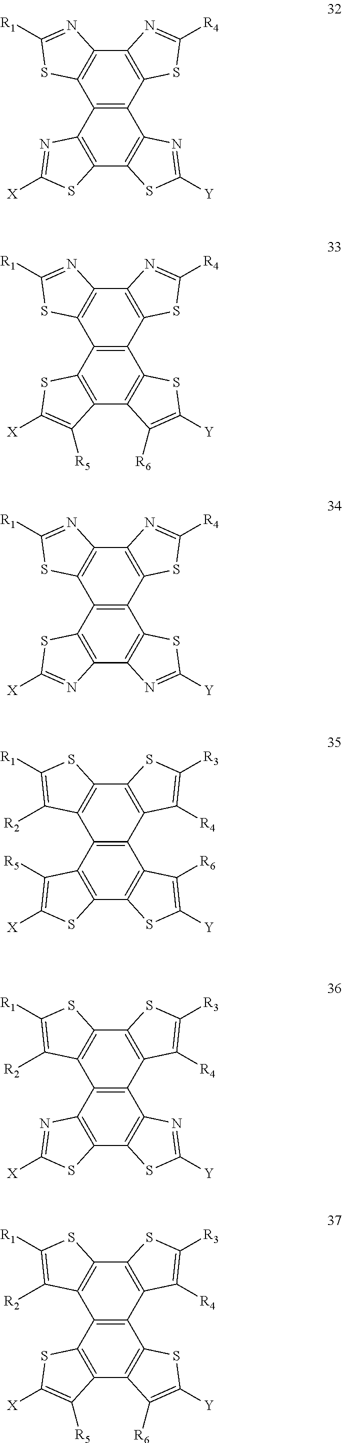

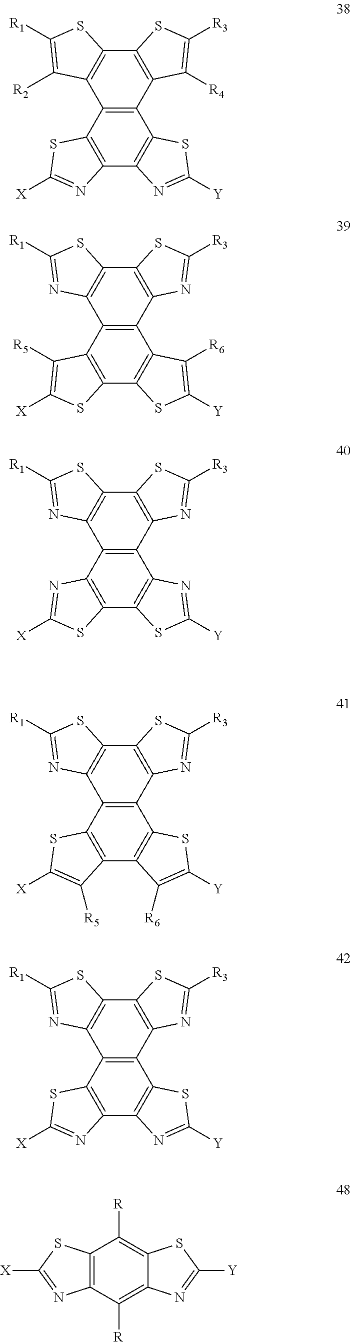

wherein each R₁, R₂, R₃, R₄, R₅, and R₆ is as given above, and X and Y are each independently selected from the group consisting of H and reactive functional groups.

In some embodiments, at least one of X and Y is a halide functional group.

In some embodiments, at least one of X and Y is either a boron functional group or a reactive tin functional group.

In some embodiments, one of X and Y is a halide functional group, and the other is either a boron functional group or a reactive tin functional group.

In some embodiments, both X and Y are halo.

In some embodiments, both X and Y are a trialkyltin.

Monomers and polymers of the present invention can be made in accordance with the techniques described herein, or variations thereof that will be apparent to those skilled in the art based upon the present disclosure. The polymers are useful for the production of microelectronic devices such as optoelectronic devices in accordance with known techniques or variations thereof that will be apparent to those skilled in the art. See, e.g., U.S. Pat. Nos. 7,534,503; and 7,348,428; US Patent Application Publication No. US 2007/0017571; PCT Patent Application No. WO 2008/000664. In some embodiments, the polymer comprises a heterojunction in the device. In some embodiments, the device comprises a first electrode, a second electrode, and a photoactive material disposed between said first and second electrode, said photoactive material comprising said polymer. Illustrative devices include, but are not limited to, a photovoltaic cell, field effect transistor, light emitting diode, photodectector, photovoltaic detector, imaging device, lasing device, storage element, amplifier, emitter, or electrochromic display

The present invention is explained in greater detail in the following non-limiting examples.

### EXPERIMENTAL

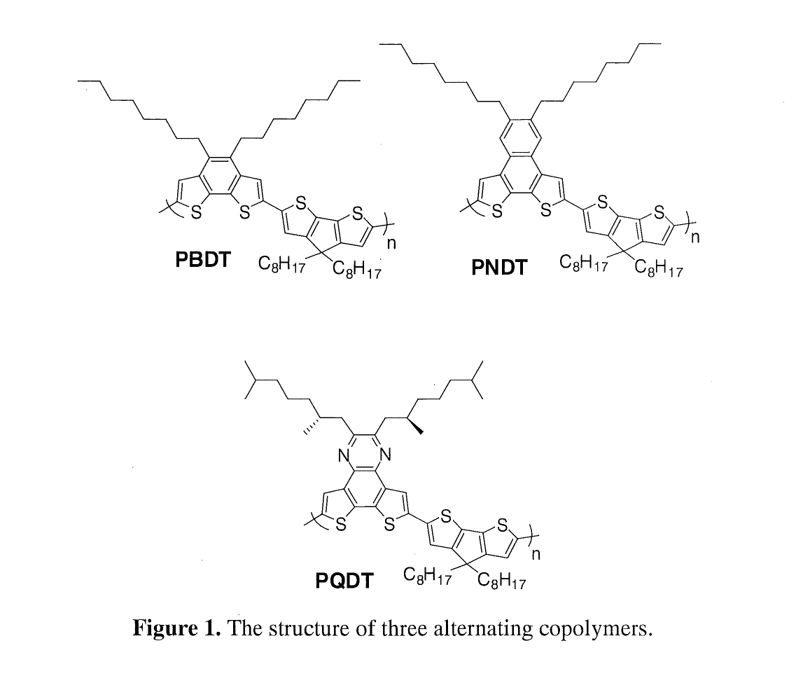

To exemplify the application of the unique features associated with these polycyclic aromatic moieties for polymer-based photovoltaics, we synthesized a family of three structurally related conjugated alternating copolymers, namely poly[2,6-(4,4-dioctyl-4H-cyclopenta[2,1-b:3,4-b′]dithiophene)-alt-2,7-(4,5-dioctylbenzo[2,1-b:3,4-b′]dithiophene)] (PBDT), poly[2,6-(4,4-dioctyl-4H-cyclopenta[2,1-b:3,4-b′]dithiophene)-alt-2,9-(5,6-dioctylnaphtho[2,1-b:3,4-b′]dithiophene)] (PNDT) and poly[2,6-(4,4-dioctyl-4H-cyclopenta[2,1-b:3,4-b′]dithiophene)-alt-6,9-(2,3-bis((S)-2,6-dimethylheptyl)dithieno[3,2-f:2′,3′-h]quinoxaline] (PQDT) (FIG. 1). The repeating units of these three copolymers consist of two modified bithiophene units with one of them planarized by bridging benzo, naphtho and quinoxalino segment, respectively. The known 2,6-(4,4-dioctyl-4H-cyclopenta)[2,1-b;3,4-b′]dithiophene moieties were introduced as the other bithiophene unit to improve the solubility of resultant copolymers, facilitating polymer characterization and photovoltaic devices fabrications. The intrinsic electronic properties of these planarized bithiophene moieties offered moderate flexibility in fine-tuning electronic properties of the corresponding copolymers. In this paper, we present the synthesis, the physical properties and the preliminary photovoltaic performances of these structurally related copolymers. The elucidated structure/property relationships will assist the intelligent exploration of future design of materials for OPV applications.

### Results and Discussion

Monomer Synthesis. To obtain benzo[2,1-b:3,4-b′]dithiophene, naphtho[2,1-b:3,4-b′]dithiophene and quinoxalino[2,1-b:3,4-b′]dithiophene moieties for the preparation of polymers PBDT, PNDT and PQDT, different synthetic strategies were applied to bridge various π systems to the bithiophene unit. Side alkyl chains were incorporated to improve solubility of resulting polymers. The synthetic route for the preparation of di-brominated monomer quinoxalino[2,1-b:3,4-b′]dithiophene 7 for PQDT is shown in Scheme 1. Quinoxalino[2,1-6:3,4-b′]dithiophene was achieved via the condensation reaction of an alkylated vicinal diamine 4 with 1,2-diketone of benzo[2,1-b:3,4-b′]bithiophene-4,5-quinone (5). 1,2-diamine 4 was obtained through multi-step synthesis from commercially available aldehyde 1. The classical acyloin condensation of aldehyde 1 followed by PCC oxidation gave alkylated 1,2-diketone 2, which was converted to 1,2-dioxime 3 and followed by Pt catalyzed hydrogenation to give the hydrogen chloride salt of 1,2-diamine in almost quantitative yield. The condensation of 1,2-diamine 4 with 1,2-diketone benzo[2,1-b:3,4-b′]bithiophene-4,5-quinone 5 under aerobic conditions directly afforded the dehydrogenated product of quinoxalino[2,1-b:3,4-b′]dithiophene 6. Di-bromination of 6 was accomplished using N-bromosuccinimide (NBS) to provide final co-monomer 7. The other co-monomer 8 was prepared by dilithiation of 4,4-dioctyl-4H-cyclopenta[2,1-b:3,4-b′]dithiophene using t-BuLi followed by quenching the intermediate with 2-isopropoxy-4,4,5,5-tetramethyl[1,3,2]dioxaborolane.

The preparation of 2,7-dibromo-4,5-dioctylbenzo[2,1-b:3,4-b′]dithiophene is depicted in Scheme 2. The synthesis was completed by a palladium catalyzed coupling reaction between 3,3′-diiodo-2,2′-bithiophene and 9-octadecyne^{22,23} followed by NBS bromination in a mixed solvent of chloroform/acetic acid.

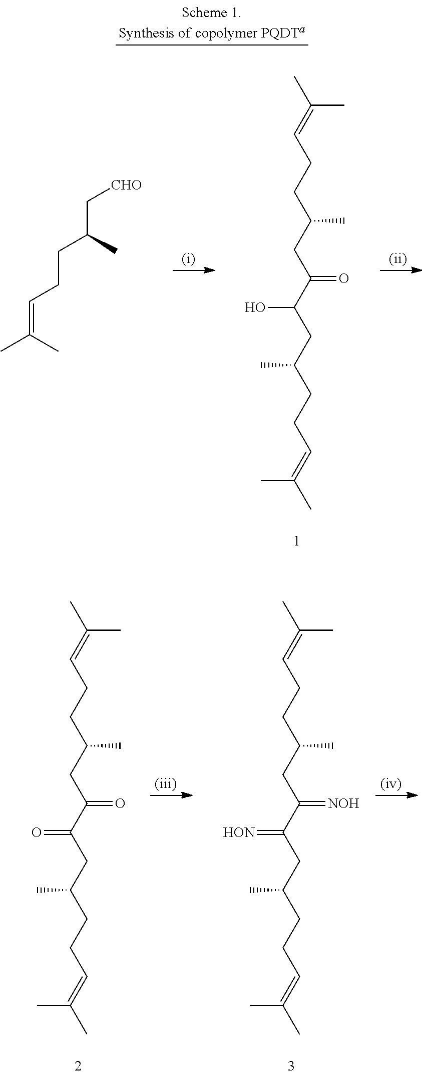

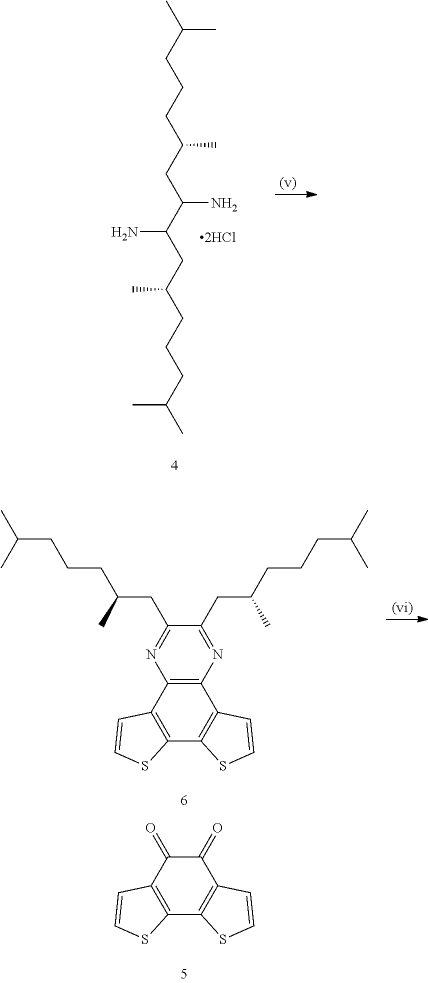

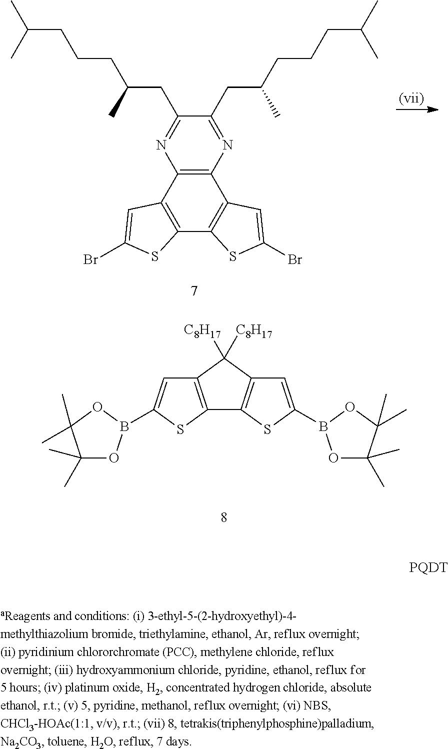

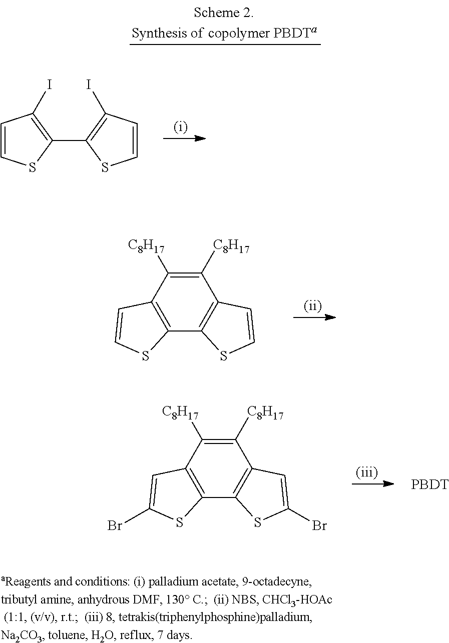

As outlined in Scheme 3, the preparation of 2,9-dibromo-5,6-dioctylnaphtho[2,1-b:3,4-b′]dithiophene started from 1,2-dichlorobenzene. A nickel-catalyzed Kumada coupling reaction between 1,2-dichlorobenzene and freshly prepared octylmagnesium bromide offered 1,2-dioctylbenzene. Iodination of 1,2-dioctylbenzene followed by palladium-catalyzed Suzuki coupling reaction with 3-thiophene boronic acid provided 4,5-bis(3-thienyl)-1,2-dioctylbenzene at high yield.¹⁸ 5,6-dioctylnaphtho[2,1-b:3,4-b′]dithiophene was then prepared via oxidative photocyclization by irradiation of a diluted toluene solution of 4,5-bis(3-thienyl)-1,2-dioctylbenzene under ambient conditions in the presence of a catalytic amount of iodine.^{24,25} Subsequent bromination using NBS in a mixed solvent of chloroform/acetic acid offered the co-monomer 2,9-dibromo-5,6-dioctylnaphtho[2,1-b:3,4-b′]dithiophene.

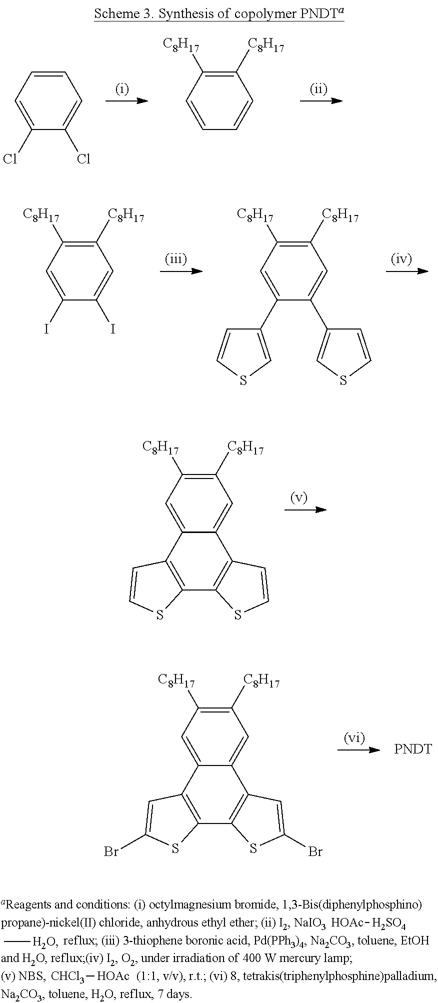

Polymer Synthesis. All copolymers were synthesized by a polycondensation of 2,6-bis(4,4,5,5-tetramethyl-1,3,2-dioxaborolan-2-yl)-4,4-dioctyl-4H-cyclopenta[2,1-b: 3,4-b′]dithiophene (8) and corresponding dibrominated co-monomers through Suzuki coupling reactions. All crude copolymers were washed successively by water and methanol and extracted by methanol and acetone successively using a Soxhlet apparatus to remove byproducts and oligomers. Finally, the polymers were extracted by chloroform, re-collected by precipitating them into methanol, and dried under vacuum. The alternating copolymers PBDT, PNDT and PQDT are soluble in common organic solvents such as methylene chloride, chloroform, THF and toluene and can be easily processed into thin films for further characterizations. The molecular structures of all alternating copolymers were confirmed by ¹H NMR spectroscopy (supporting information).

The yields and molecular weights of three copolymers are listed in Table 1. High polymer yields (˜90%) were obtained from Suzuki-coupling polymerizations. The molecular weights were determined by gel permeation chromatography (GPC) in THF using polystyrene standards. Thermogravimetric analysis (TGA) and differential scanning calorimetry (DSC) analysis revealed that all polymers did not degrade below 430° C. nor did they melt (supporting information).

TABLE 1
Polymerization results for polymers PBDT, PNDT and PQDT.

| | Yield^{a} | M_{w}^{b} | Mₙ^{b} | | T_{d}^{c} |
|---|---|---|---|---|---|
| | [%] | [kg/mol] | [kg/mol] | PDI^{b} | [° C.] |
| PBDT | 88 | 39.1 | 20.6 | 1.90 | 432 |
| PNDT | 90 | 25.8 | 16.6 | 1.54 | 432 |
| PQDT | 86 | 30.9 | 16.1 | 1.91 | 430 |

^{a}Soluble polymers extracted by CHCl₃ with respect to the overall yield.
^{b}Determined by GPC in THF using polystyrene standards.
^{c}The temperature of degradation corresponding to a 5% weight loss determined by TGA at a heating rate of 10° C./min.

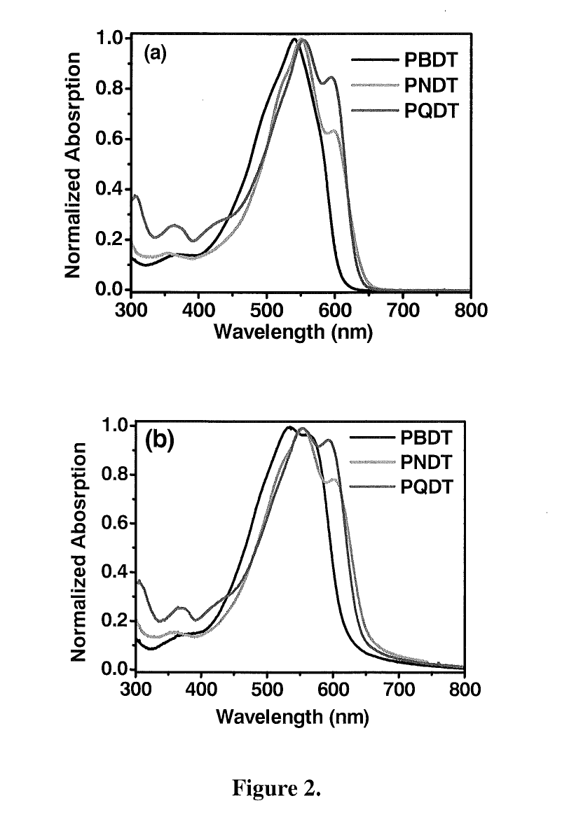

Optical Absorption. The electronic absorption data of the three alternating copolymers are listed in Table 2. All spectroscopic properties were measured both in toluene solutions (FIG. 2a) and as thin films on glass slides (FIG. 2b). As shown in FIG. 2a, PNDT and PQDT have almost identical absorption maxima at 552 nm, which is 15 nm red-shifted compared to that of PBDT. The low energetic edge of the absorption spectrum of individual polymer was used to approximate the band gap of corresponding polymer. The band gap of PBDT was estimated to be 2.06 eV (absorption edge: ˜600 nm), while a smaller band gap of 1.96 eV was calculated for PNDT and PQDT (absorption edge: ˜631 nm). Such a decrease in the band gap can be explained by the fact that the naphthalene and quinoxalene units provide more conjugation than the benzene unit when incorporated into the bithiophene unit in the conjugated backbone of copolymers. A similar behavior was observed for the absorption spectra of the three polymers at thin films (FIG. 2b). Unexpectedly, only a tiny red shift (less than 5 nm) was observed for the absorptions from solution to thin film for all copolymers, which suggests less inter-chain stacking induced by π-π interaction.¹⁶ The negligible absorption shift between solution and thin film of three copolymers may be caused by the two octyl groups in 4H-cyclopenta[2,1-b:3,4-b′]-dithiophene moiety which imparts steric hindrance and affects the planarity of the conjugated backbone.

Electrochemistry. Cyclic voltammetry (CV) was employed to investigate the electrochemical properties of the three copolymers and to determine the energy levels of individual copolymers. Cyclic voltammograms of the oxidation and reduction behaviors (supporting information) were recorded from thin films of PBDT, PNDT and PQDT drop-casted from chloroform solutions as described in the experimental section. The potentials were internally calibrated using the ferrocene/ferrocenium redox couple (Fc/Fc⁺) which has a known reduction potential of 4.8 eV.^{26,27} The highest occupied molecular orbital (HOMO) and lowest unoccupied molecular orbital (LUMO) energy levels of copolymers were calculated from the onset oxidation potentials (Eₒₙₑₛₜ^{ox}) and onset reductive potentials (Eₒₙₑₛₜ^{red}), respectively, according to equation (1) and (2). The electrochemically determined band gaps were deduced from the difference between onset potentials from oxidation and reduction of copolymers as depicted in equation (3).

$$\begin{matrix}HOMO = - ( {E}_{onest}^{ox} + 4.8 ) ( eV ) & ( 1 ) \\ LUMO = - ( {E}_{onest}^{ox} + 4.8 ) ( eV ) & ( 2 ) \\ {E}_{gap}^{EC} = {E}_{onest}^{ox} - {E}_{onest}^{red} & ( 3 )\end{matrix}$$

The CV data of three copolymers are presented in Table 2. The band gap of PNDT or PQDT with bridged naphtho or quinoxalino segment to bithiophene moiety showed a decrease of ca. 0.1 eV compared to that of PBDT with bridged benzo segment. This behavior is consistent with the results from UV-Vis absorption spectra. However, the HOMO energy level of PNDT remained unchanged as compared to that of PBDT (−5.04 eV). Compared with PBDT and PNDT, PQDT showed a decrease of ca. 0.1 eV in its HOMO energy level (−5.15 eV). The LUMO energy level of PQDT also decreased about 0.1 eV accordingly to maintain a band gap of 2.10 eV. The noticeably lower HOMO and LUMO levels in the case of PQDT, are ascribed to the two nitrogen atoms in the planarized π system, because these two nitrogen atoms render the resulting conjugated molecule more electron-deficient. From these results, we conclude that bridging different π segments with intrinsically different electronic properties to the bithiophene moieties allows a moderate modulation of the band gap and energy level of resulting polymers. This finding will assist future design of semiconductive polymers with tunable electronic properties towards OPV applications.

TABLE 2
Optical and electrochemical data of the polymers PBDT, PNDT, PQDT

| | Uv-Vis absorption data | | | | | | Cyclic Voltammetry | | |
|---|---|---|---|---|---|---|---|---|---|
| | toluene solution | | | film | | | Eₒₙₑₛₜ^{ox} | Eₒₙₑₛₜ^{red} | |
| | λₘₐₓ | λₒₙₛₑₜ | E_{g} ^{a} | λₘₐₓ | λₒₙₛₑₜ | E_{g} ^{a} | HOMO | LUMO | E_{gap}^{EC} |
| polymer | [nm] | [nm] | [eV] | [nm] | [nm] | [eV] | [V/eV] | [V/eV] | [eV] |
| PBDT | 538 | 602 | 2.06 | 538 | 617 | 2.00 | 0.24/−5.04 | −1.97/−2.83 | 2.21 |
| PNDT | 552 | 631 | 1.96 | 555 | 650 | 1.91 | 0.24/−5.04 | −1.86/−2.94 | 2.10 |
| PQDT | 554 | 631 | 1.96 | 555 | 641 | 1.94 | 0.35/−5.15 | −1.75/−3.05 | 2.10 |

^{a} Calculated from the intersection of the tangent on the low energetic edge of the absorption spectrum with the abscissa.

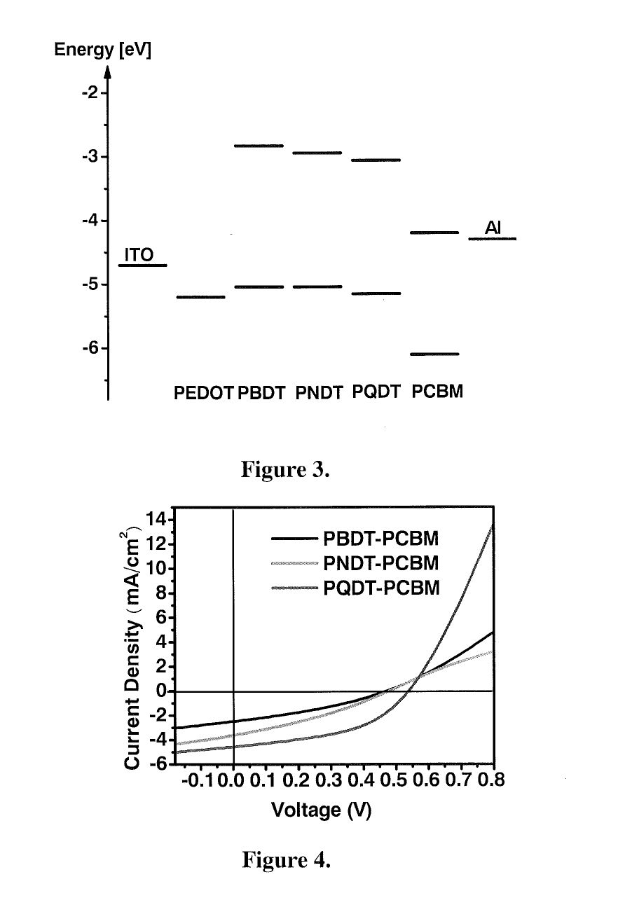

Photovoltaic Properties. PCBM as the electron accepting component has been widely used in OPV devices. FIG. 3 exhibits a diagram of energy levels of three alternating copolymers in relation to that of PCBM, and the work functions of indium tin oxide (ITO), poly(3,4-ethylenedioxythiophene) poly(styrenesulfonate) (PEDOT:PSS) and aluminum (Al) used as electrodes in an OPV device. The LUMO energy levels of three copolymers are distinctively higher than that of PCBM. The difference between the LUMO energy levels of three copolymers and PCBM is over 1.2 eV, which is sufficiently high to enable an unrestricted and directed charge transfer.²⁸ Thus, all three copolymers were applied as donors into a conventional BHJ type OPV device with PCBM as acceptor in order to investigate the effect of different bridging π segments within the bithiophene moiety on the photovoltaic properties. Hole mobility values for all copolymers were estimated via space-charge limit current (SCLC) by fabricating a hole-only device according to Blom's device configuration^{29,30} as detailed in the experimental section. The hole mobilities were found to be 3.01×10⁻⁵ cm² V⁻¹ S⁻¹, 1.3×10⁻⁵ cm² V⁻¹ S⁻¹, 5.15×10⁻⁵ cm² V⁻¹ S⁻¹ for PBDT, PNDT, and PQDT respectively.

Typical I-V characteristics of ITO/PEDOT:PSS/copolymer:PCBM (1:1.6, w/w)/Al devices are depicted in FIG. 4 under AM 1.5G irradiation (100 mW/cm²). The devices with PBDT:PCBM layers (90 nm) showed an open circuit voltage (V_{oc}) of 0.47 V, a short circuit current density (J_{sc}) of 2.47 mA/cm², and a fill factor (FF) of 0.32, giving an energy conversion efficiency (η) of 0.38%. The V_{oc} value is close to the difference (0.82 V) between the HOMO energy level of PBDT and LUMO energy level of PCBM after the correction for an expected voltage loss of around 0.2 V at each electrode due to band bending.³¹ The devices with PNDT:PCBM blends (90 nm) demonstrated a Vₒ, value of 0.47 V, a J_{sc} value of 3.61 mA/cm², a FF of 0.33, leading to the η value of 0.55%, a slightly improved performance relative to PBDT. The same V_{oc} value of PBDT and PNDT based devices can be explained by the identical HOMO energy levels of both polymers (Table 2). Although the hole mobility of PBDT is slightly higher (3.01×10⁻⁵ cm² V⁻¹ S⁻¹) than that of PNDT (1.3×10⁻⁵ cm² V⁻¹ S⁻¹), the broader absorption of PNDT than that of PBDT resulted in higher short circuit current and thus the slightly improved overall efficiency for PNDT based devices. For the BHJ devices made from PQDT:PCBM films (100 nm), the devices exhibited an increased V_{oc} value of 0.53 V, also an increased J_{sc} value of 4.56 mA/cm² and an improved FF of 0.47, resulting in the significantly improved energy conversion efficiency of 1.14%. The increased V_{oc} value of PQDT based devices is expected since PQDT has a lower HOMO energy level (−5.15 eV) than that of PBDT and PNDT (−5.04 eV). The increased current relative to that of PNDT is ascribed mainly to the fact that PQDT has higher hole mobility than that of PNDT since both polymers have the same band gap (2.10 eV). Tapping-mode atomic force microscopy (AFM) studies were carried out to investigate the film morphology of polymer:PCBM blends on their photovoltaic performances. Rough surfaces and blend phase separation were observed for PBDT:PCBM and PNDT:PCBM films (supporting information) compared to relatively smooth surface and more intimate mixing for PQDT:PCBM layer, which somehow explained better hole mobility of PQDT in devices over PBDT and PNDT. The improved miscibility of PQDT and PCBM, together with the higher V_{oc} value and smaller band gap, leads to the improved overall energy conversion efficiency.

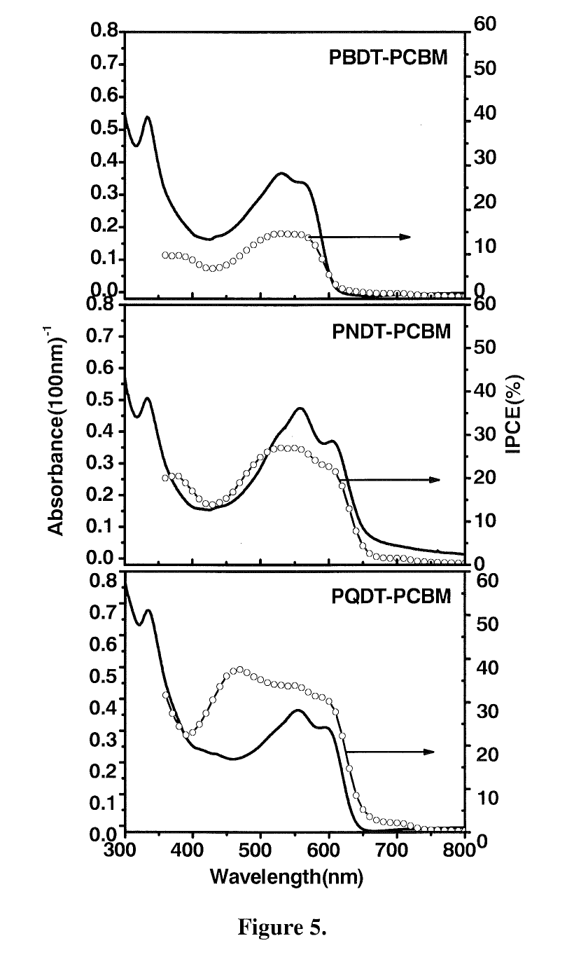

The incident-photon-to-current efficiency (IPCE) spectra of the photovoltaic devices from copolymer:PCBM blends are presented in FIG. 5 together with the absorption of thin films from copolymer: PCBM blends. The IPCE spectra of PBDT and PNDT match the optical absorptions well and show the maximum of 15% at 538 nm for PBDT and 27% at 555 nm for PNDT, respectively. For PBDT, a broad plateau around the maximum in IPCE spectrum exists between 500 and 580 nm, while occurring between 480 and 620 nm for PNDT. This phenomenon was caused by the stronger and wider absorption of PNDT:PCBM blend between 450 and 700 nm than that for PBDT:PCBM at the same film thickness. A similar match is found between the absorption spectrum and the IPCE spectrum for PQDT:PCBM films. The IPCE spectrum shows a maximum of 37% at 460 nm and an average value of 33% in the absorption area from 430 to 620 nm for devices based on PQDT:PCBM films. The higher IPCE value over the entire absorption wavelength region further explained the improved photovoltaic performance of PQDT:PCBM over the blends of the other two copolymers with PCBM.

Conclusions. We have successfully synthesized three alternating copolymers based on 4H-cyclopenta[2,1-b:3,4-b′]dithiophene as the common unit, while employing different structurally related conjugated units, namely, benzo[2,1-b:3,4-b′]dithiophene (PBDT), naphtho[2,1-b:3,4-b′]dithiophene (PNDT) and quinoxalino[2,1-b:3,4-b′]dithiophene (PQDT). By bridging intrinsically different π system to bithiophene moiety to obtain enhanced π-electron delocalization and incorporating them into semiconductive alternating copolymers, the band gap, the HOMO and LUMO energy levels of resulting copolymers can be fine-tuned as demonstrated from the investigation of optical absorption properties and electrochemical studies of PBDT, PNDT and PQDT. The three copolymers were applied as electron-donating materials with PCBM as acceptor in conventional BHJ photovoltaic devices. A peak IPCE value of 37% and an overall power conversion efficiency of 1.14% was obtained from a PQDT/PCBM blend device, which is very encouraging given the quite large band gap of 2.1 eV for PQDT. Although the energy conversion efficiencies for these un-optimized photovoltaic devices are still not sufficiently high, this study enriched our understanding of tuning the electronic properties of conjugated semiconductive polymers for photovoltaic applications and provided further insights for future materials design.

### Experimental Section

### Reagents and Instrumentation

All reagents and chemicals were purchased from commercial sources (Aldrich, Acros, Strem, Fluka) and used without further purification unless stated otherwise. Reagent grade solvents were dried when necessary and purified by distillation. Melting points were uncorrected. Elemental analysis was carried out at the Atlantic Microlab. Gel permeation chromatography (GPC) measurements were performed on a Waters 2695 Separations Module apparatus with a differential refractive index detector with tetrahydrofuran (THF) as eluent. The obtained molecular weight is relative to the polystyrene standard. Thermogravimetric analysis (TGA) measurements were carried out with a PerkinElmer thermogravimetric analyzer (Pyris 1 TGA) at a heating rate of 10° C. min⁻¹ under a nitrogen atmosphere. The temperature of degradation (T_{d}) is correlated to a 5% weight loss. Differential scanning calorimetry (DSC) analyses were recorded on a DSC220C instrument from SII Seiko Instruments. ¹H nuclear magnetic resonance (NMR) measurements were recorded either with a Bruker Avance 300 MHz AMX or Bruker 400 MHz DRX spectrometer. ¹³C nuclear magnetic resonance (NMR) measurements were carried out with a Bruker 400 MHz DRX spectrometer. Chemical shifts were expressed in parts per million (ppm), and splitting patterns are designated as s (singlet), d (doublet), t (triplet) and m (multiplet). Coupling constants J are reported in Hertz (Hz). The mass spectroscopy was carried out on Micromass Quattro II Triple Quadrupole Mass Spectrometer. 3,3′-diiodo-2,2′-bithiophene,²² 4,4-dioctyl-4H-cyclopenta[2,1-b:3,4-b′]dithiophene,³² and benzo[2,1-b:3,4-b′]bithiophene-4,5-quinone (5)^{33,34} were synthesized according to literature procedures.

Electrochemistry. Cyclic voltammetry measurements were carried out using a Bioanalytical Systems (BAS) Epsilon potentiostat equipped with a standard three-electrode configuration. Typically, a three electrodes cell equipped with a glassy carbon working electrode, a Ag/AgNO₃ (0.01M in anhydrous acetonitrile) reference electrode, and a Pt wire counter electrode was employed. The measurements were done in anhydrous acetonitrile with tetrabutyl ammonium hexafluorophosphate (0.1 M) as the supporting electrolyte under an argon atmosphere at a scan rate of 100 mV/s. Polymer films were drop cast onto the glassy carbon working electrode from a 2.5 mg/mL chloroform solution and dried under house nitrogen stream prior to measurements. The potential of Ag/AgNO₃ reference electrode was internally calibrated by using the ferrocene/ferrocenium redox couple (Fc/Fc⁺). The electrochemical onsets were determined at the position where the current starts to differ from the baseline.

Spectroscopy. UV-Visible absorption spectra were obtained by a Shimadzu UV-2401PC spectrophotometer. Fluorescence spectra were recorded on a Shimadzu RF-5301PC spectrofluorophotometer. For the measurements of thin films, polymers were spin-coated onto pre-cleaned glass slides from 10 mg/mL polymer solutions in chlorobenzene.

AFM. Tapping mode with a Nanoscope III AFM (Digital Instruments, Inc., Santa Barbara, Calif.), The measurements were performed at ambient conditions (in air, 20° C.) using Si cantilevers with a spring constant of −50 N/m, a tip radius of 8 nm, and a resonance frequency of about 300 kHz.

Polymer solar cell fabrication and testing. Glass substrates coated with patterned indium-doped tin oxide (ITO) were purchased from Thin Film Devices, Inc. The 150 nm sputtered ITO pattern had a resistivity of 15Ω/□. Prior to use, the substrates were ultrasonicated for 10 minutes in deionized water followed by the rinse with deionized water and the treatment in acetone and then 2-propanol in the same way. The substrates were dried under a stream of nitrogen and subjected to the treatment of UV-Ozone over 20 minutes. A filtered dispersion of PEDOT:PSS in water (Baytron-PH500) was then spin-coated onto clean ITO substrates under 4000 rpm for 60 seconds and then baked at 130° C. for 15 minutes to give a thin film with a thickness of 45 nm. A blend of polymer and PCBM (1:1.6 w/w, 10 mg/mL for polymers) was dissolved in chlorobenzene with heating at 60° C. for 2 hours, filtered through a 0.45 μm poly(tetrafluoroethylene) (PTFE) filter, spin-coated at 1200 rpm for 60 seconds onto PEDOT:PSS layer. The substrates were then dried under vacuum at room temperature for 12 hours. The thicknesses of films were recorded by a profilometer (Alpha-Step 200, Tencor Instruments). The devices were finished for measurement after thermal deposition of 100 nm aluminum film as the cathode at a pressure of ˜1×10⁻⁶ mbar. There are 8 devices per substrate, with an active area of 18 mm² per device. Device characterization was carried out under AM 1.5G irradiation with the intensity of 100 mW/m² (Oriel 91160, 300 W) calibrated by a NREL certified standard silicon cell. Current versus potential (I-V) curves were recorded with a Keithley 2400 digital source meter. IPCE were detected under monochromatic illumination (Oriel Cornerstone 260¼ m monochromator equipped with Oriel 70613NS QTH lamp) and the calibration of the incident light was performed with a monocrystalline silicon diode. All fabrication steps after adding the PEDOT:PSS layer onto ITO substrate, and characterizations were performed in gloveboxes under nitrogen atmosphere. For mobility measurements, the hole-only devices in a configuration of ITO/PEDOT:PSS (45 nm)/copolymer-PCBM (1:1.6, w/w)/Pd (40 nm) were fabricated. The experimental dark current densities J of polymer: PCBM blends were measured when applied with voltage from 0 to 6V. The applied voltage V was corrected from the built-in voltage V_{bi}³⁰ which was taken as a compensation voltage V_{bi}=V_{oc}+0.05 V and the voltage drop Vᵣₛ across the indium tin oxide/poly(3,4-ethylene-dioxythiophene):poly(styrene sulfonic acid) (ITO/PEDOT:PSS) series resistance and contact resistance, which is found to be around 35Ω from a reference device without the polymer layer. From the plots of J^{0.5} vs. V (supporting information), hole mobilities of copolymers can be deduced from³⁵

$$J = \frac{9}{8} {ɛ}_{r} {ɛ}_{0} {μ}_{h} \frac{{V}^{2}}{{L}^{3}} \qquad ( 4 )$$

where ε₀ is the permittivity of free space, εᵣ is the dielectric constant of the polymer which is assumed to be around 3 for the conjugated polymers in our experiment,³⁶ μₕ is the hole mobility, V is the voltage drop across the device, and L is the film thickness of active layer.

### Synthesis

(6S,11S)-9-hydroxy-2,6,11,15-tetramethylhexadeca-2,14-dien-8-one (1).³⁷ To a 250 mL of two-necked round-botton(RB) flask containing (−)citronellal (25.0 g, 163 mmol) in 50 mL of ethanol under argon was added the catalyst of 3-ethyl-5-(2-hydroxyethyl)-4-methylthiazolium bromide (4.1 g, 16.3 mmol) and triethylamine (17.0 mL, 120 mmol). The mixture was then heated to reflux over night. After removal of the solvent under reduced pressure, the resulted mixture was poured into 100 mL of water and extracted by ethyl ether (3×60 mL). The combined organic layer was dried over anhydrous MgSO₄ and concentrated under vacuum. The residue was purified by flash chromatography on silica gel (hexane:ethyl acetate=20:1, v/v) to afford 18.5 g of product as a colorless oil (yield: 75%). ¹H NMR (400 MHz, CDCl₃) δ 5.08 (m, 2H), 4.15 (m, 1H), 3.47 (dd, 1H, J=5.04 Hz), 2.40 (m, 1H), 2.2 (m, 1H), 1.9-2.1 (m, 6H), 1.60 (d, 12H), 1.1-1.3 (m, 4H), 0.8-1.0 (m, 6H). ¹³C NMR (400 MHz, CDCl₃) δ 212.63, 131.63, 131.30, 124.52, 124.40, 124.00, 75.59, 74.70, 45.16, 45.15, 41.15, 41.00, 37.95, 36.85, 36.83, 35.56, 29.36, 29.04, 28.92, 28.81, 25.65, 25.42, 25.39, 25.36, 25.22, 20.29, 19.82, 19.65, 18.46, 17.60.

(6S,11S)-2,6,11,15-tetramethylhexadeca-2,14-diene-8,9-dione (2). To a solution of 1 (7.0 g, 22.7 mmol) in 100 mL of methylene chloride was added 7.5 g of PCC. The mixture was heated to reflux. After 16 hours, the mixture was cooled to room temperature and filtered. The solution was concentrated under reduced pressure. The crude compound was purified by flash chromatography on silica gel (hexane:ethyl acetate=20:1, v/v) to afford the product as a colorless oil. Yield: 3.5 g (50%). ¹H NMR (400 MHz, CDCl₃) δ 5.06 (m, 2H), 2.66-2.74 (dd, 2H, J=5.64, 16.71 Hz), 2.51-2.60 (dd, 2H, J=7.98, 16.73 Hz), 1.92-2.02 (m, 6H), 1.5-1.67 (s, 6H), 1.58 (s, 6H), 1.19-1.35 (m, 4H), 0.89 (d, 6H, J=6.66 Hz). ¹³C NMR (400 MHz, CDCl₃) δ 200.05, 131.60, 124.07, 52.96, 36.94, 28.49, 25.66, 25.36, 19.71, 17.60.

(6S,11S)-2,6,11,15-tetramethylhexadeca-2,14-diene-8,9-dione dioxime (3). A 250 mL of two-necked RB flask containing a solution of 2 (6.12 g, 20.0 mmol) in ethanol (60 mL) and pyridine (8.0 mL) was purged with argon. Hydroxyammonium chloride (7.0 g, 100.0 mmol) was then added in one portion. The mixture was heated to reflux for 5 hours. After removal of the solvent under reduced pressure, 100 mL of water/ethanol (2:1, v/v) was added and ultrasonicated before filtration. The solid was then rinsed by 20 mL of cold hexane and dried under vacuum to afford a white pure solid. Yield: 5.5 g (95%). mp: 131-131.6° C. ¹H NMR (400 MHz, CD₃OD) δ 4.93 (m, 2H), 3.16 (m, 2H), 2.32-2.47 (m, 4H), 1.72-1.90 (m, 6H), 1.51 (s, 6H), 1.44 (s, 6H), 1.17-1.20 (m, 2H), 1.01-1.05 (m, 2H), 0.72 (d, 6H, J=6.7 Hz). ¹³C NMR (400 MHz, CD₃OD) δ 157.95, 131.66, 126.05, 38.51, 32.19, 31.42, 26.68, 25.86, 20.16, 17.68.

(6S,11S)-2,6,11,15-tetramethylhexadecane-8,9-diamine dihydrogen chloride (4). To a solution of 3 (1.9 g) in 50 mL of absolute ethanol at room temperature was added platinum oxide (0.4 g) and 2.0 mL of concentrated hydrogen chloride. The mixture was then purged with hydrogen and was kept stirring under hydrogen (with a hydrogen balloon) over 10 hours. After removing the solvent under reduced pressure, the residue was rinsed with cold hexane and directly used in the next step without further purification.

2,3-bis((S)-2,6-dimethylheptyl)dithieno[3,2-f:2′,3′-h]quinoxaline (6). To a 100 mL of two-necked RB flask equipped with a condenser was added the solution of 4 (1.20 g) in 50 mL of methanol, 5 (0.66 g, 3 mmol) and 2.0 mL of pyridine. The mixture was then heated to reflux with stirring over night. After removing the solvent under reduced pressure, the residue was re-dissolved in 30 mL of methylene chloride and washed by water and dried over anhydrous MgSO₄. The organic layer was then concentrated and the residue was purified by flash chromatography on silica gel (hexane:methylene chloride=4:1, v/v) to afford 0.91 g of pure product as a white solid (yield: 60%). mp: 57.5-58.7° C. ¹H NMR (300 MHz, CDCl₃) δ 8.31 (d, 2H, J=5.28 Hz), 7.51 (d, 2H, J=5.22 Hz), 2.94-3.16 (dd, 2H, J=6.08 Hz, 14.2 Hz), 2.87-2.90 (dd, 2H, J=8.0 Hz, 14.2 Hz), 2.33 (m, 2H), 1.55 (m, 2H), 1.2-1.52 (m, 12H), 1.03 (d, 6H, J=6.58 Hz). 0.88 (d, 12H, J=7.45 Hz). ¹³C NMR (400 MHz, CDCl₃) δ 154.06, 136.04, 135.11, 133.95, 124.12, 42.13, 39.23, 37.36, 32.91, 27.98, 24.87, 22.71, 22.59, 19.82. MS: m/z=496.6 [M+2H]⁺ (Calcd.: 494.3)

6,9-dibromo-2,3-bis((S)-2,6-dimethylheptyl)dithieno[3,2-f:2′,3′-h]quinoxaline (7). To a solution of 6 (0.99 g, 2.0 mmol) in 20 mL of chloroform/acetic acid (1:1, v/v) at room temperature was added NBS (0.75 g, 4.2 mmol). The resulting mixture was stirred at room temperature for 24 hours and then diluted by 50 mL of water. The organic layer was washed by 5% sodium hydroxide solution, water and brine and dried over anhydrous MgSO₄. After removing the solvent, the crude product was further purified by flash chromatography on silica gel (hexane:methylene chloride=5:1, v/v) to afford 0.85 g of pure product as a white solid (yield: 65%). mp: 106.4-107.9° C. ¹H NMR (300 MHz, CDCl₃) δ 8.15 (s, 2H), 3.00-3.07 (dd, 2H, J=6.05 Hz, 14.38 Hz), 2.78-2.85 (dd, 2H, J=7.96 Hz, 14.38 Hz), 2.28 (m, 2H), 1.55 (m, 2H), 1.17-1.45 (m, 12H), 0.98 (d, 6H, J=6.60 Hz). 0.88 (d, 12H, J=6.58 Hz). ¹³C NMR (400 MHz, CDCl₃) δ 155.12, 135.13, 134.73, 133.60, 126.73, 112.97, 42.17, 39.27, 37.39, 32.73, 28.03, 24.88, 22.73, 22.61, 19.86. MS: m/z=652.6 [M+2H]⁺ (Calcd.: 650.1). Anal. Calcd. for C₃₀H₄₀Br₂N₂S₂: C, 55.21; H, 6.18; Br, 24.49; N, 4.29; S, 9.83. Found: C, 55.49; H, 6.24; Br, 24.59; N, 4.18; S, 9.78.

2,6-Bis(4,4,5,5-tetramethyl-1,3,2-dioxaborolan-2-yl)-4,4-dioctyl-4H-cyclopenta[2,1-b:3,4-b′]dithiophene (8). A solution of 4,4-dioctyl-4H-cyclopenta[2,1-b:3,4-b′]dithiophene (1.2 g, 3.0 mmol) in 20 mL of dry THF under argon was cooled to −78° C., and n-BuLi in hexane (2.5 M, 4.8 mL, 12 mmol) was added over 10 min with stirring. The mixture was kept at −78° C. for another 1 hour before 2-isopropoxy-4,4,5,5-tetramethyl[1,3,2]dioxaborolane (3.2 mL, 25 mmol) was added. The cooling bath was removed after 3 hours and the mixture was allowed to warm to room temperature overnight (16 h). After subsequent dilution with ethyl ether and washing with brine and large amount of water, the organic layer was dried over anhydrous MgSO₄, and concentrated under reduced pressure to give a solid which was washed further with cold methanol and dried in vacuo to afford the pure product as a pale yellow solid (1.4 g, 71%). nip: 109-110° C. ¹H NMR (300 MHz, CDCl₃): δ 7.42 (s, 2H), 1.79 (m, 4H), 1.36 (s, 24H), 1,1-1.24 (m, 10H), 0.90-0.93 (m, 4H), 0.84 (t, 6H, J=7.08 Hz). ¹³C NMR (400 MHz, CDCl₃): δ 161.41, 143.84, 131.04, 83.97, 52.74, 37.79, 31.75, 29.99, 29.24, 24.76, 24.46, 22.56, 14.02. MS: m/z=654.6 [M]⁺ (Calcd.: 654.4). Anal. Calcd. for C₃₇H₆₀B₂O₄S₂: C, 67.89; H, 9.24; S, 9.80. Found: C, 68.05; H, 9.41; S, 9.72.

4,5-dioctylbenzo[2,1-b:3,4-b′]dithiophene.²³ To a two-necked RB flask under nitrogen was added 1.39 g (3.3 mmol) of 3,3′-diiodo-2,2′-bithiophene, 222 mg (0.33 mmol) of Pd(OAc)₂, 2.5 g (10 mmol) of 9-octadecyne, tributyl amine 1.85 g (10 mmol), and 10 mL of anhydrous DMF. The mixture was heated at 130° C. for 4 hours. After cooling down to room temperature, 50 mL of ether ethyl was. The organic phase was washed with water several times, dried by MgSO₄, concentrated under reduced pressure. The residue was further purified by flash chromatography on silica gel (hexane as eluent) to afford the pure product as a colorless liquid (1.16 g, yield: 85%). ¹H NMR (300 MHz, CDCl₃) δ 7.46 (d, 2H, J=5.43 Hz), 7.37 (d, 2H, J=5.42 Hz), 3.01 (t, 4H, J=7.89 Hz), 1.68 (m, 4H), 1.51 (m, 4H), 1.30 (m, 16H), 0.89 (t, 6H, J=6.7 Hz). ¹³C NMR (400 MHz, CDCl₃) δ 137.47, 131.58, 131.32, 123.55, 123.38, 31.87, 31.52, 30.43, 30.16, 29.48, 29.27, 22.63, 14.04. MS: m/z=414.7 [M]⁺ (Calcd.: 414.7).

2,7-dibromo-4,5-dioctylbenzo[2,1-b:3,4-b′]dithiophene. To a solution of 4,5-dioctylbenzo[2,1-b:3,4-b′]dithiophene (0.83 g, 2 mmol) in 10 mL of chloroform/glacial acetic acid (1:1, v/v) was added 0.72 g of NBS at room temperature. After the reaction was completed, 20 mL of chloroform was added. The mixture was then washed by water, 5% NaOH and brine. After drying with anhydrous MgSO₄, the organic phase was concentrated and the residue was further purified by flash chromatography on silica gel (hexane as eluent) to give 0.8 g final product (yield: 70%). mp: 63.8-65.2° C. ¹H NMR (300 MHz, CDCl₃) δ 7.39 (s, 2H), 2.88 (t, 4H, J=8.25 Hz), 1.58 (m, 4H), 1.48 (m, 4H), 1.32 (m, 16H), 0.89 (t, 6H, J=6.9 Hz). ¹³C NMR (400 MHz, CDCl₃) δ 137.28, 131.46, 131.40, 126.26, 112.53, 31.89, 31.41, 30.36, 30.10, 29.44, 29.28, 22.67, 14.08. MS: m/z=572.6 [M]⁺ (Calcd.: 572.5). Anal. Calcd. for C₂₆H₃₆Br₂S₂: C, 54.55; H, 6.34; Br, 27.91; S, 11.20. Found: C, 54.75; H, 6.32; Br, 27.89; S, 11.35.

1,2-dioctylbenzene. The synthesis of 1,2-dioctylbenzene and 4,5-dioctyl-1,2-diiodobenzene was adopted from the reported procedure.³⁸ A flame dried, 250 mL of three-necked RB flask equipped with a condenser and an addition funnel was loaded with magnesium metal turnings (12.0 g, 0.48 mol) in 20 mL of anhydrous ethyl ether under an argon atmosphere. A solution of 1-bromooctane (80.5 mL, 0.46 mol) in 50 mL of anhydrous ethyl ether was added dropwise in a rate that a gentle reflux was maintained. After the addition of the bromide solution, the resulting mixture was heated under reflux for additional 2 hours. After cooling to room temperature, the clear solution of the Grignard reagent was transferred through a cannula into a flame dried addition funnel and added dropwise to a stirred solution of 1,2-dichlorobenzene (23 mL, 0.2 mol) and 1,3-bis(diphenylphosphino)propane)-nickel(II) chloride (0.7 g, 1.29 mmol) in 50 mL of anhydrous ethyl ether at room temperature. The reaction mixture was then heated to reflux overnight and then cooled to room temperature and poured into 200 mL of hydrochloric acid (2 M) with ice. The organic layer was separated and washed with water, Na₂CO₃, brine and water and dried over anhydrous MgSO₄. After removing the solvent under reduced pressure, the residue was purified by passing through a short silica gel column (hexane as eluent). The distillation of the resulting oil under reduced pressure gave 39.0 g of pure 1,2-dioctyllbenzene (138° C./0.25 mmHg) as a colorless liquid (yield: 65%). ¹H NMR (400 MHz, CDCl₃) δ 7.12 (m, 4H), 2.59 (t, 4H, J=7.76 Hz), 1.56 (m, 4H), 1.27-1.37 (m, 20H), 0.88 (t, 6H, J=6.36 Hz). ¹³C NMR (400 MHz, CDCl₃) δ 140.56, 129.08, 125.66, 32.69, 31.90, 31.35, 29.81, 29.51, 29.28, 22.68, 14.11. MS: m/z=302.3 [M]⁺ (Calcd.: 302.3).

4,5-dioctyl-1.2-diiodobenzene. 1,2-Dioctylbenzene (12.3 g, 40.6 mol) was added to a RB flask loaded with glacial acetic acid (150 mL), H₂SO₄ (concd, 9.0 mL), H₂O (1 mL), NaIO₃ (4.016 g, 20.3 mmol), and I₂ (11.34 g, 44.7 mmol) at room temperature. The resulting mixture was then heated under reflux overnight. After cooling to room temperature, a saturated aqueous Na₂S₂O₄ solution was added until the color of the mixture changed from purple to light brown. The mixture was then extracted with CH₂Cl₂ (50 mL×3), washed with saturated Na₂S₂O₄, H₂O, and brine, and then dried over anhydrous Na₂SO₄. After removing the solvent under reduced pressure, the brownish residue was purified by flash chromatography on silica gel to afford a colorless oil (18.7 g, yield: 83%). ¹H NMR (400 MHz, CDCl₃) δ 7.60 (s, 2H), 2.46 (t, 4H, J=8.0 Hz), 1.52 (m, 4H), 1.27-1.37 (m, 20H), 0.88 (t, 6H, J=7.04 Hz). ¹³C NMR (400 MHz, CDCl₃) δ 142.62, 139.69, 103.97, 31.87, 31.80, 30.85, 29.54, 29.35, 29.16, 22.60, 14.05. MS: m/z=554.1 [M]⁺ (Calcd.: 554.1).

4,5-Bis(3-thienyl)-1,2-dioctylbenzene.¹⁸ To a three-necked 250 mL RB flask equipped with a condenser was added 4,5-dioctyl-1.2-diiodobenzene (11.08 g, 20 mmol), 3-thiophene boronic acid (6.4 g, 50.0 mmol), Na₂CO₃ (24.0 g, 226 mmol) in a mixed solvent of toluene (50 mL), EtOH (50 mL), and H₂O (50 mL). The resulting mixture was vigorously stirred during the cycle of evacuation/refilling with argon three times. The catalyst Pd(PPh₃)₄ (1% equiv., 575 mg, 0.54 mmol) was then added to the mixture under a gentle argon stream and the system was heated at reflux overnight. After cooling to room temperature, the reaction mixture was diluted with 100 mL of ethyl ether and the aqueous layer was removed. The organic layer was washed with water, brine and dried with anhydrous MgSO₄. After the solvent removal under reduced pressure, the residue was purified by flash chromatography on silica gel (hexane:ethyl acetate=20:1, v/v) to provide 7.2 g of the product (yield: 78%). ¹H NMR (400 MHz, CDCl₃) δ 7.25 (s, 2H), 7.17 (dd, 2H, J=4.92 Hz, 2.98 Hz), 7.05 (dd, 2H, J=1.22 Hz, 2.97 Hz), 6.80 (dd, 2H, J=1.23 Hz, 4.94 Hz), 2.66 (t, 4H, J=7.80 Hz), 1.65 (m, 4H), 1.30-1.50 (m, 20H), 0.90 (t, 6H, J=6.96 Hz). ¹³C NMR (400 MHz, CDCl₃) δ 142.20, 140.03, 132.65, 130.92, 129.08, 124.39, 122.38, 32.45, 31.91, 31.40, 29.88, 29.51, 29.29, 22.68, 14.13. MS: m/z=466.3 [M]⁺ (Calcd.: 466.2).

5,6-Dioctylnaphtho[2,1-b:3,4-b′]dithiophene. In a quartz tube was added a solution of 4,5-bis(3-thienyl)-1,2-dioctylbenzene (0.5 g, 1.1 mmol) and iodine (30 mg) in toluene (500 mL). The system was then irradiated by a 400 W mercury lamp equipped with an efficient cooling system for 16 hours under magnetic stirring and air bubbling. The reaction mixture was washed with a saturated aqueous solution of Na₂S₂O₃, dried over MgSO₄ and concentrated. The residue was purified by flash chromatography on silica gel (hexane as eluent), and a white solid was obtained (0.33 g, yield: 65%). mp: 62.2-63.1° C. ¹H NMR (400 MHz, CDCl₃) δ 8.11 (s, 2H), 7.97 (d, 2H, J=5.34 Hz), 7.47 (d, 2H, J=5.30), 2.86 (t, 4H, J=7.76 Hz), 1.74 (m, 4H), 1.32-1.50 (m, 20H), 0.92 (t, 6H, J=6.80 Hz). ¹³C NMR (400 MHz, CDCl₃) δ 139.22, 134.12, 131.25, 126.09, 124.04, 123.34, 122.74, 33.14, 31.29, 31.52, 29.86, 29.58, 29.34, 22.70, 14.14. MS: m/z=464.4 [M]⁺ (Calcd.: 464.2).

2,9-dibromo-5,6-dioctylnaphtho[2,1-b:3,4-b′]dithiophene. To a stirred solution of 5,6-dioctylnaphtho[2,1-b:3,4-b′]dithiophene (1 g, 2.16 mmol) in a mixture of chloroform-acetic acid (1/1, v/v, 10 mL) at room temperature was added NBS (0.773 g, 4.3 mmol). The resulting solution was stirred overnight. The mixture was poured into 100 mL of water and extracted with chloroform (50 mL). The combined organic layer was further washed with 5% aqueous NaOH solution, brine and water, dried with MgSO₄, and was concentrated under reduced pressure. The residue was purified by flash chromatography on silica gel to give 0.92 g of the pure product (yield: 68%). mp: 105.3-106.4° C. ¹H NMR (400 MHz, CDCl₃) δ 7.84 (s, 2H), 7.80 (d, 2H), 2.79 (m, 4H, J=7.63 Hz), 1.70 (m, 4H), 1.31-1.48 (m, 20H), 0.91 (t, 6H, J=6.91 Hz). ¹³C NMR (400 MHz, CDCl₃) δ 139.84, 133.84, 130.90, 125.51, 124.90, 123.64, 111.98, 33.08, 31.93, 31.31, 29.88, 29.57, 29.35, 22.71, 14.14. MS: m/z=620.2 [M]⁺ (Calcd.: 620.0). Anal. Calcd. for C₃₀H₃₃Br₂S₂: C, 57.88; H, 6.15; Br, 25.67; S, 10.30. Found: C, 57.97; H, 6.20; Br, 25.84; S, 10.38.

Synthesis of alternating copolymers via Suzuki coupling polymerization. A representative procedure is as follows. To a flame dried 25 mL of two-necked RB flask equipped with a condenser was added 7 (195.8 mg, 0.3 mmol), 8 (196.4 mg, 0.3 mmol), 6.0 mL of 2 M Na₂CO₃, 10 mL of toluene, 2 drops of Aliquat 336 under a gentle argon stream with vigorous stirring. The resulting mixture was evacuated and refilled with argon for three cycles to remove oxygen and then was added Pd(PPh₃)₄ (17 mg, 0.015 mmol, 5% equiv.) under argon stream. The mixture was heated under reflux over 7 days. After cooling to room temperature, the organic layer was separated and washed by water. Addition of 100 mL of methanol to organic solution offered the precipitate, which was collected by filtration and successively washed with water and methanol and dried under air. The crude polymer was then extracted subsequently with methanol, acetone, and chloroform in a Soxhlet extractor. The fraction from chloroform was concentrated under reduced pressure and precipitated into methanol to give the polymer PQDT as a blue solid (0.23 g, 86%). ¹H NMR (400 MHz, CDCl₃): δ 7.79 (2H), 7.32 (2H), 3.08 (2H), 2.86 (2H), 2.32 (2H), 1.98 (4H), 1.20-1.80 (H), 0.95 (6H), 0.85 (6H).

PBDT yield: 0.23 g (88%). ¹H NMR (400 MHz, CDCl₃) δ 7.46 (2H), 7.17 (2H), 2.98 (4H), 1.92 (4H), 1.80-1.22 (48H), 0.93 (6H), 0.85 (6H).

PNDT yield: 0.22 g (90%). ¹H NMR (400 MHz, CDCl₃) δ 7.97 (2H), 7.85 (2H), 7.38 (2H), 2.85 (4H), 2.01 (4H), 1.9-1.2 (48H), 0.98-0.7 (12H).

### REFERENCES

(1) Ma, W. L.; Yang, C. Y.; Gong, X.; Lee, K.; Heeger, A. J. Adv. Funct. Mater. 2005, 15, 1617-1622.
(2) Li, G.; Shrotriya, V.; Huang, J. S.; Yao, Y.; Moriarty, T.; Emery, K.; Yang, Y. Nature Mater. 2005, 4, 864-868.
(3) Thompson, B. C.; Frechet, J. M. J. Angew. Chem. Int. Ed. 2008, 47, 58-77.
(4) Scharber, M. C.; Wuhlbacher, D.; Koppe, M.; Denk, P.; Waldauf, C.; Heeger, A. J.; Brabec, C. L. Adv. Mater. 2006, 18, 789-794.
(5) Brabec, C. J.; Winder, C.; Sariciftci, N. S.; Hummelen, J. C.; Dhanabalan, A.; van Hal, P. A.; Janssen, R. A. J. Adv. Funct. Mater. 2002, 12, 709-712.
(6) Muhlbacher, D.; Scharber, M.; Morana, M.; Zhu, Z. G.; Waller, D.; Gaudiana, R.; Brabec, C. Adv. Mater. 2006, 18, 2884-2889.
(7) Peet, J.; Kim, J. Y.; Coates, N. E.; Ma, W. L.; Moses, D.; Heeger, A. J.; Bazan, G. C. Nature Mater. 2007, 6, 497-500.
(8) Zhang, F. L.; Jespersen, K. G.; Bjorstrom, C.; Svensson, M.; Andersson, M. R.; Sundstrom, V.; Magnusson, K.; Moons, E.; Yartsev, A.; Inganas, O. Adv. Funct. Mater. 2006, 16, 667-674.
(9) Andersson, L. M.; Zhang, F. L.; Inganas, O. Appl. Phys. Lett. 2007, 91, 071108/1-071108/3.
(10) Slooff, L. H.; Veenstra, S. C.; Kroon, J. M.; Moet, D. J. D.; Sweelssen, J.; Koetse, M. M. Appl. Phys. Lett. 2007, 90, 143506/1-143506/3.
(11) Wienk, M. M.; Turbiez, M. G. R.; Struijk, M. P.; Fonrodona, M.; Janssen, R. A. J. Appl. Phys. Lett. 2006, 88, 153511/1-153511/3.
(12) Yao, Y.; Shi, C. J.; Li, G.; Shrotriya, V.; Pei, Q. B.; Yang, Y. Appl. Phys. Lett. 2006, 89, 153507/1-153507/3.
(13) Ashraf, R. S.; Shahid, M.; Klemm, E.; Al-Ibrahim, M.; Sensfuss, S. Macromol. Rapid Commun. 2006, 27, 1454-1459.
(14) Blouin, N.; Michaud, A.; Leclerc, M. Adv. Mater. 2007, 19, 2295-2300.
(15) Blouin, N.; Michaud, A.; Gendron, D.; Wakim, S.; Blair, E.; Neagu-Plesu, R.; Belletete, M.; Durocher, G.; Tao, Y.; Leclerc, M. J. Am. Chem. Soc. 2008, 130, 732-742.
(16) Roncali, J. Chem. Rev. 1997, 97, 173-205.
(17) Tovar, J. D.; Rose, A.; Swager, T. M. J. Am. Chem. Soc. 2002, 124, 7762-7769.
(18) Tovar, J. D.; Swager, T. M. Adv. Mater. 2001, 13, 1775-1780.
(19) Polycyclic hydrocarbons I and II; Clar, E. Ed. Academic Press: London 1964.
(20) Watson, M. D.; Fechtenkotter, A.; Müllen, K. Chem. Rev. 2001, 101, 1267-1300.
(21) Shklyarevskiy, I. O.; Jonkheijm, P.; Stutzmann, N.; Wasserberg, D.; Wondergem, H. J.; Christianen, P. C. M.; Schenning, A.; de Leeuw, D. M.; Tomovic, Z.; Wu, J. S.; Müllen, K.; Maan, J. C. J. Am. Chem. Soc. 2005, 127, 16233-16237.
(22) Miura, M.; Satoh, T.; Watanabe, H.; Ueda, M. WO/2007/105638, 2007.
(23) Watanabe, H.; Kumagai, J.; Tsurugi, H.; Satoh, T.; Miura, M. Chem. Lett. 2007, 36, 1336-1337.
(24) Jayasuriya, N.; Kagan, J.; Owens, J. E.; Kornak, E. P.; Perrine, D. M. J. Org. Chem. 1989, 54, 4203-4205.
(25) Nicolas, Y.; Blanchard, P.; Levillain, E.; Allain, M.; Mercier, N.; Roncali, J. Org. Lett. 2004, 6, 273-276.
(26) Pommerehne, J.; Vestweber, H.; Guss, W.; Mahrt, R. F.; Bassler, H.; Porsch, M.; Daub, J. Adv. Mater. 1995, 7, 551-554.
(27) Zhan, X. W.; Liu, Y. Q.; Wu, X.; Wang, S. A.; Zhu, D. B. Macromolecules 2002, 35, 2529-2537.
(28) Arkhipov, V. I.; Bassler, H. Phys. Status Solidi A 2004, 201, 1152-1187.
(29) Melzer, C.; Koop, E. J.; Mihailetchi, V. D.; Blom, P. W. M. Adv. Funct. Mater. 2004, 14, 865-870.
(30) Mihailetchi, V. D.; Koster, L. J. A.; Blom, P. W. M.; Melzer, C.; de Boer, B.; van Duren, J. K. J.; Janssen, R. A. J. Adv. Funct. Mater. 2005, 15, 795-801.
(31) Mihailetchi, V. D.; Blom, P. W. M.; Hummelen, J. C.; Rispens, M. T. J. Appl. Phys. 2003, 94, 6849-6854.
(32) Coppo, P.; Cupertino, D. C.; Yeates, S. G.; Turner, M. L. J. Mater. Chem. 2002, 12, 2597-2599.
(33) Wynberg, H.; Sinnige, H. J. M. Rec. Tray. Chim. 1969, 88, 1244-1245.
(34) Ohnishi, H.; Kozaki, M.; Okada, K. Synth. Met. 2003, 135, 85-86.
(35) Goodman, A. M.; Rose, A. J. Appl. Phys. 1971, 42, 2823-2830.
(36) Goh, C.; Kline, R. J.; McGehee, M. D.; Kadnikova, E. N.; Fréchet, J. M. J. Appl. Phys. Lett. 2005, 86, 122110-122113.
(37) Popp, F. D. J. Heterocyclic Chem. 1974, 11, 79-82.
(38) Zhou, Q.; Carroll, P. J.; Swager, T. M. J. Org. Chem. 1994, 59, 1294-1301.

### ADDITIONAL EXPERIMENTAL

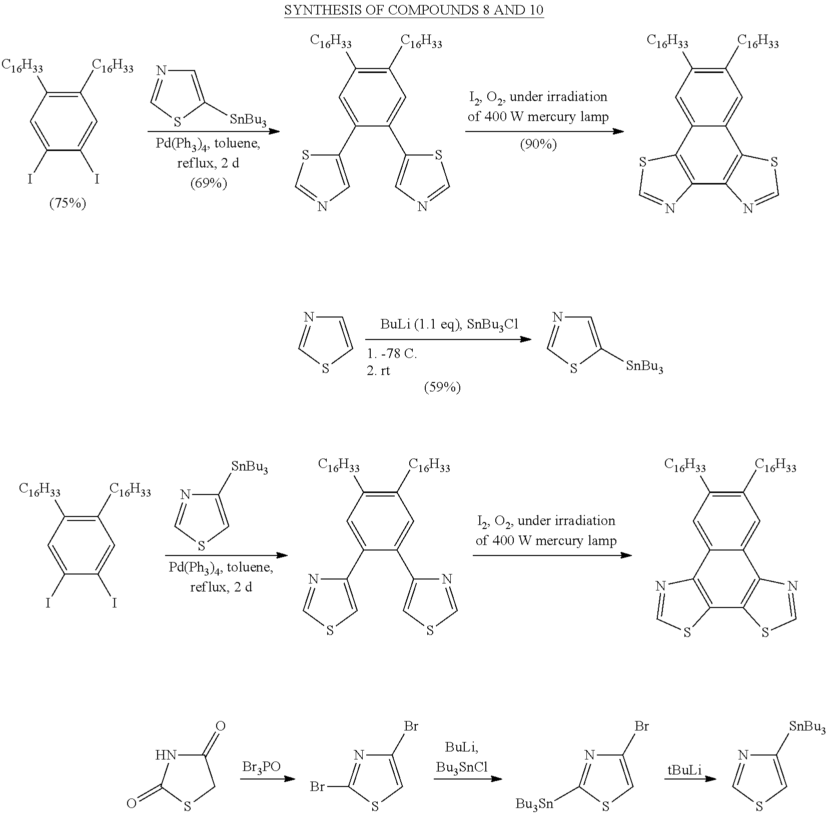

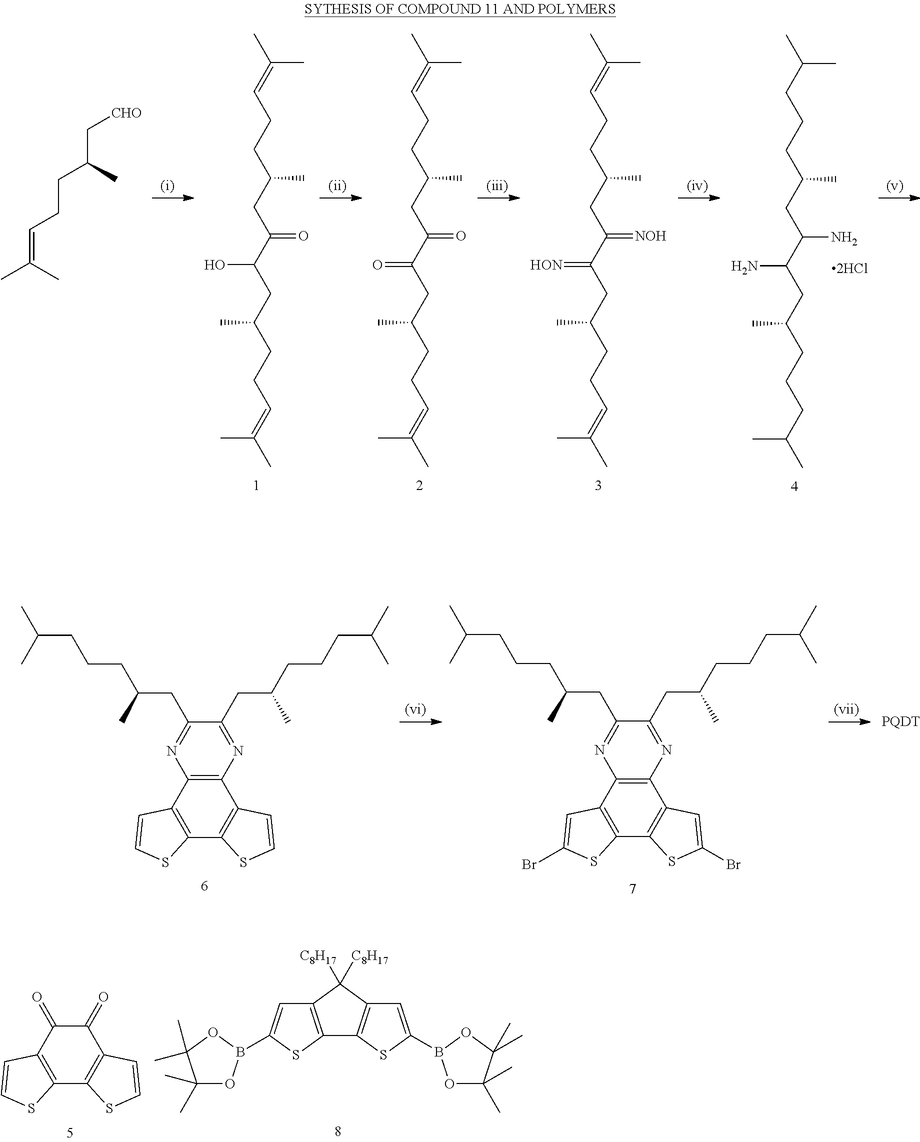

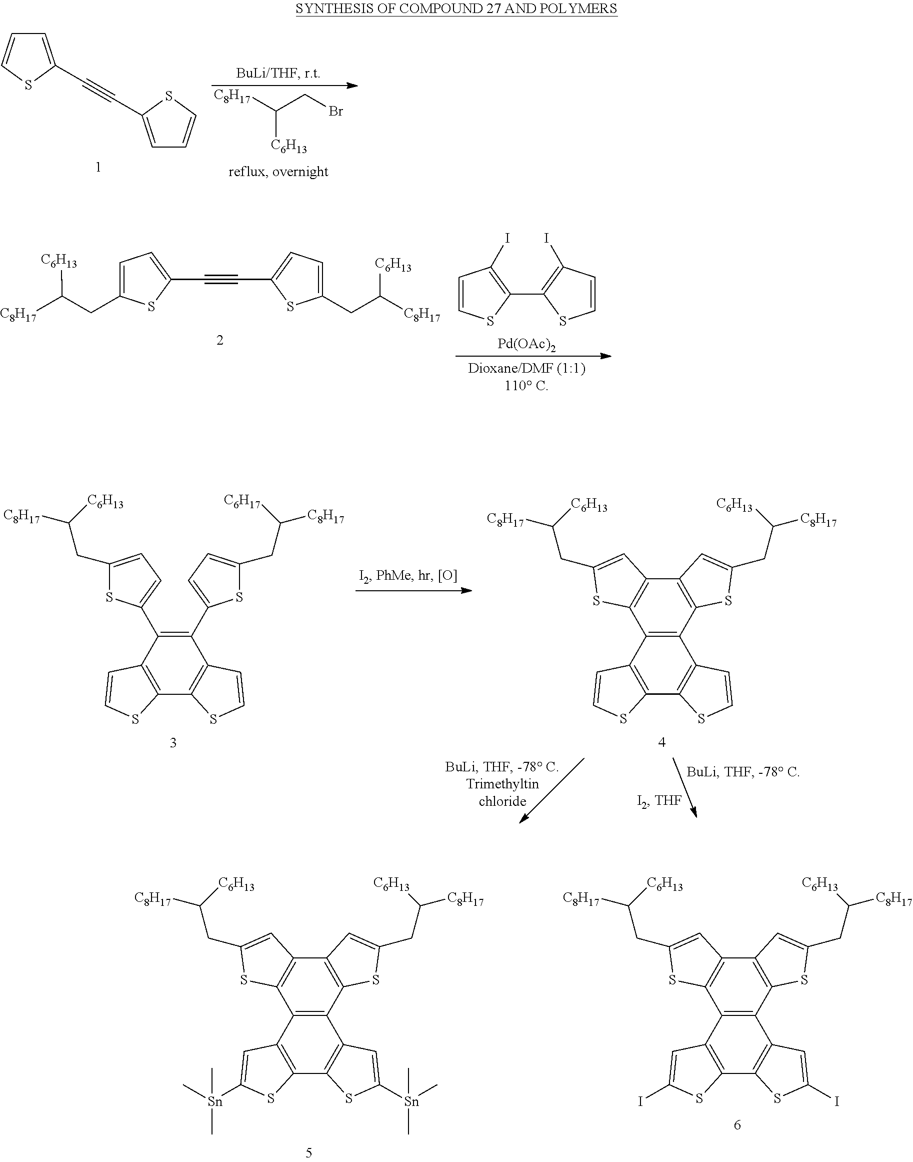

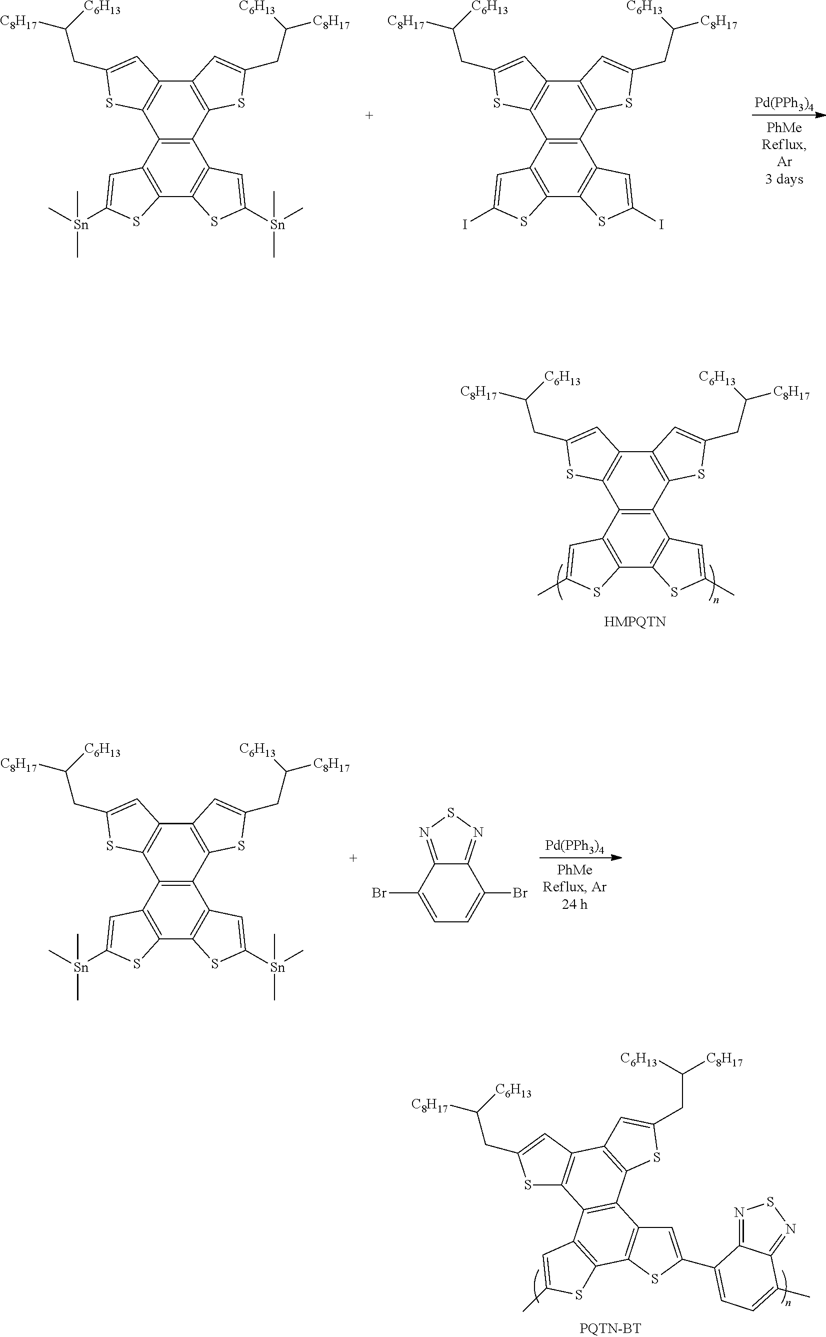

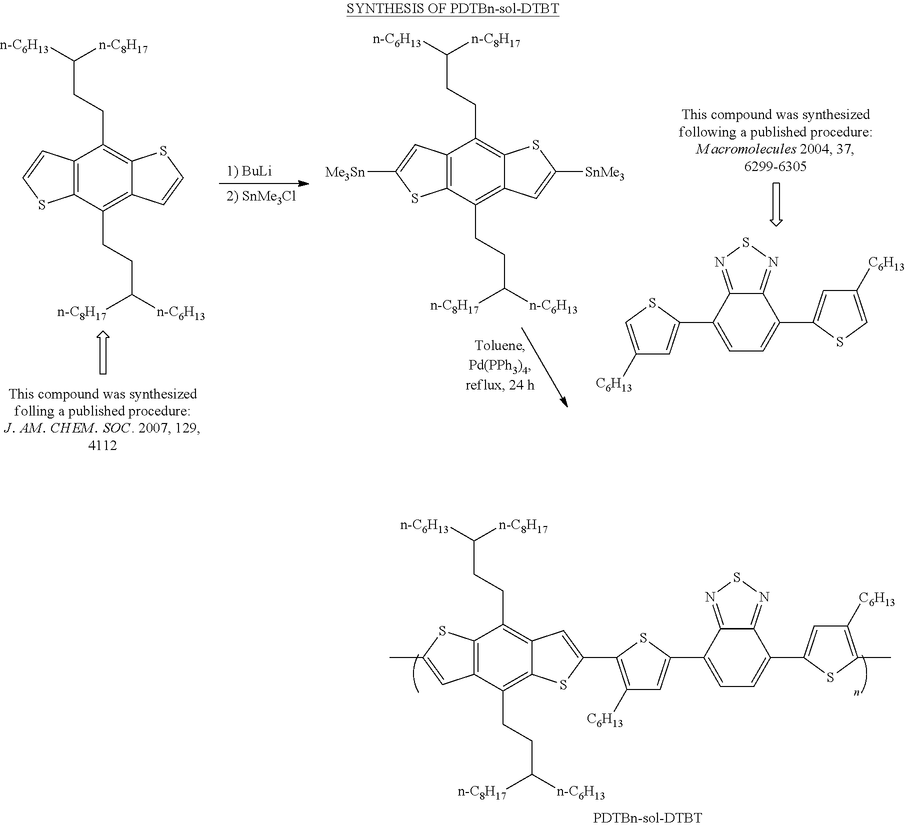

### Synthesis of Compound 11 and Polymers

(6S,11S)-9-hydroxy-2,6,11,15-tetramethylhexadeca-2,14-dien-8-one (1). To a 250 mL of two-necked round-botton(RB) flask containing (−)citronellal (25.0 g, 163 mmol) in 50 mL of ethanol under argon was added the catalyst of 3-ethyl-5-(2-hydroxyethyl)-4-methylthiazolium bromide (4.1 g, 16.3 mmol) and triethylamine (17.0 mL, 120 mmol). The mixture was then heated to reflux over night. After removal of the solvent under reduced pressure, the resulted mixture was poured into 100 mL of water and extracted by ethyl ether (3×60 mL). The combined organic layer was dried over anhydrous MgSO₄ and concentrated under vacuum. The residue was purified by flash chromatography on silica gel (hexane:ethyl acetate=20:1, v/v) to afford 18.5 g of product as a colorless oil (yield: 75%). ¹H NMR (400 MHz, CDCl₃) δ 5.08 (m, 2H), 4.15 (m, 1H), 3.47 (dd, 1H, J=5.04 Hz), 2.40 (m, 1H), 2.2 (m, 1H), 1.9-2.1 (m, 6H), 1.60 (d, 12H), 1.1-1.3 (m, 4H), 0.8-1.0 (m, 6H). ¹³C NMR (400 MHz, CDCl₃) δ 212.63, 131.63, 131.30, 124.52, 124.40, 124.00, 75.59, 74.70, 45.16, 45.15, 41.15, 41.00, 37.95, 36.85, 36.83, 35.56, 29.36, 29.04, 28.92, 28.81, 25.65, 25.42, 25.39, 25.36, 25.22, 20.29, 19.82, 19.65, 18.46, 17.60.

(6S,11S)-2,6,11,15-tetramethylhexadeca-2,14-diene-8,9-dione (2). To a solution of 1 (7.0 g, 22.7 mmol) in 100 mL of methylene chloride was added 7.5 g of PCC. The mixture was heated to reflux. After 16 hours, the mixture was cooled to room temperature and filtered. The solution was concentrated under reduced pressure. The crude compound was purified by flash chromatography on silica gel (hexane:ethyl acetate=20:1, v/v) to afford the product as a colorless oil. Yield: 3.5 g (50%). ¹H NMR (400 MHz, CDCl₃) δ 5.06 (m, 2H), 2.66-2.74 (dd, 2H, J=5.64, 16.71 Hz), 2.51-2.60 (dd, 2H, J=7.98, 16.73 Hz), 1.92-2.02 (m, 6H), 1.5-1.67 (s, 6H), 1.58 (s, 6H), 1.19-1.35 (m, 4H), 0.89 (d, 6H, J=6.66 Hz). ¹³C NMR (400 MHz, CDCl₃) δ 200.05, 131.60, 124.07, 52.96, 36.94, 28.49, 25.66, 25.36, 19.71, 17.60.

(6S,11S)-2,6,11,15-tetramethylhexadeca-2,14-diene-8,9-dione dioxime (3). A 250 mL of two-necked RB flask containing a solution of 2 (6.12 g, 20.0 mmol) in ethanol (60 mL) and pyridine (8.0 mL) was purged with argon. Hydroxyammonium chloride (7.0 g, 100.0 mmol) was then added in one portion. The mixture was heated to reflux for 5 hours. After removal of the solvent under reduced pressure, 100 mL of water/ethanol (2:1, v/v) was added and ultrasonicated before filtration. The solid was then rinsed by 20 mL of cold hexane and dried under vacuum to afford a white pure solid. Yield: 5.5 g (95%). mp: 131-131.6° C. ¹H NMR (400 MHz, CD₃OD) δ 4.93 (m, 2H), 3.16 (m, 2H), 2.32-2.47 (m, 4H), 1.72-1.90 (m, 6H), 1.51 (s, 6H), 1.44 (s, 6H), 1.17-1.20 (m, 2H), 1.01-1.05 (m, 2H), 0.72 (d, 6H, J=6.7 Hz). ¹³C NMR (400 MHz, CD₃OD) δ 157.95, 131.66, 126.05, 38.51, 32.19, 31.42, 26.68, 25.86, 20.16, 17.68.

(6S,11S)-2,6,11,15-tetramethylhexadecane-8,9-diamine dihydrogen chloride (4). To a solution of 3 (1.9 g) in 50 mL of absolute ethanol at room temperature was added platinum oxide (0.4 g) and 2.0 mL of concentrated hydrogen chloride. The mixture was then purged with hydrogen and was kept stirring under hydrogen (with a hydrogen balloon) over 10 hours. After removing the solvent under reduced pressure, the residue was rinsed with cold hexane and directly used in the next step without further purification.

2,3-bis((S)-2,6-dimethylheptyl)dithieno[3,2-f:2′,3′-h]quinoxaline (6). To a 100 mL of two-necked RB flask equipped with a condenser was added the solution of 4 (1.20 g) in 50 mL of methanol, 5 (0.66 g, 3 mmol) and 2.0 mL of pyridine. The mixture was then heated to reflux with stirring over night. After removing the solvent under reduced pressure, the residue was re-dissolved in 30 mL of methylene chloride and washed by water and dried over anhydrous MgSO₄. The organic layer was then concentrated and the residue was purified by flash chromatography on silica gel (hexane:methylene chloride=4:1, v/v) to afford 0.91 g of pure product as a white solid (yield: 60%). mp: 57.5-58.7° C. ¹H NMR (300 MHz, CDCl₃) δ 8.31 (d, 2H, J=5.28 Hz), 7.51 (d, 2H, J=5.22 Hz), 2.94-3.16 (dd, 2H, J=6.08 Hz, 14.2 Hz), 2.87-2.90 (dd, 2H, J=8.0 Hz, 14.2 Hz), 2.33 (m, 2H), 1.55 (m, 2H), 1.2-1.52 (m, 12H), 1.03 (d, 6H, J=6.58 Hz). 0.88 (d, 12H, J=7.45 Hz). ¹³C NMR (400 MHz, CDCl₃) δ 154.06, 136.04, 135.11, 133.95, 124.12, 42.13, 39.23, 37.36, 32.91, 27.98, 24.87, 22.71, 22.59, 19.82. MS: m/z=496.6 [M+2H]⁺ (Calcd.: 494.3)

Synthesis of alternating copolymers via Suzuki coupling polymerization. A representative procedure is as follows. To a flame dried 25 mL of two-necked RB flask equipped with a condenser was added 7 (195.8 mg, 0.3 mmol), 8 (196.4 mg, 0.3 mmol), 6.0 mL of 2 M Na₂CO₃, 10 mL of toluene, 2 drops of Aliquat 336 under a gentle argon stream with vigorous stirring. The resulting mixture was evacuated and refilled with argon for three cycles to remove oxygen and then was added Pd(PPh₃)₄ (17 mg, 0.015 mmol, 5% equiv.) under argon stream. The mixture was heated under reflux over 7 days. After cooling to room temperature, the organic layer was separated and washed by water. Addition of 100 mL of methanol to organic solution offered the precipitate, which was collected by filtration and successively washed with water and methanol and dried under air. The crude polymer was then extracted subsequently with methanol, acetone, and chloroform in a Soxhlet extractor. The fraction from chloroform was concentrated under reduced pressure and precipitated into methanol to give the polymer PQDT as a blue solid (0.23 g, 86%). ¹H NMR (400 MHz, CDCl₃): δ 7.79 (2H), 7.32 (2H), 3.08 (2H), 2.86 (2H), 2.32 (2H), 1.98 (4H), 1.20-1.80 (H), 0.95 (6H), 0.85 (6H).

### Synthesis of Compound 27 and Polymers

Synthesis of compound 4 (27). In a 1000 mL of round bottom flask was added a solution of compound 3 (2.0 g, 2.5 mmol) and iodine (100 mg) in toluene (800 mL). The mixture was then put into the irradiation from a 400 W mercury lamp equipped with an efficient cooling system for 16 hours under magnetic stirring and air bubbling. The reaction mixture was then washed with a saturated aqueous solution of Na₂S₂O₃, dried over MgSO₄ and concentrated. After chromatography on silica gel (eluent: hexane), a colorless liquid was obtained (1.1 g, yield: 55%). ¹H NMR (300 MHz, CDCl₃) δ 8.64 (d, 2H, J=5.51 Hz), 7.64 (d, 2H, J=5.51 Hz), 7.52 (s, 2H), 3.01 (d, 4H, J=6.71 Hz), 1.88 (m, 2H), 1.40-1.20 (m, 48H), 0.88-0.83 (m, 12H). ¹³C NMR (400 MHz, CDCl₃) 145.01, 133.68, 132.92, 132.70, 132.62, 126.03, 123.46, 121.30, 120.24, 40.14, 35.25, 35.16, 33.46, 31.93, 31.60, 30.00, 29.68, 29.65, 29.35, 26.71, 26.88, 22.69, 14.14.

Synthesis of compound 5 Compound 4 (0.8 g, 1.0 mmol) was dissolved in dry THF (30 mL) under argon at room temperature. 2.5M of n-BuLi in hexane (0.84 mL, 2.1 mmol) was added dropwise. After stirring at room temperature for 20 minutes, trimethyltin chloride (1 M in hexanes, 3 mL, 3 mmol) was injected in by a syringe. The reaction was then quenched by 20 mL of water ten minutes later. The mixture was extracted with ethyl ether. The organic layer was washed with water several times and dried over anhydrous magnesium sulfate. After removal of the solvent, the residue was dried under high vacuum to afford 0.8 g of pure product (yield 70%) as a pale yellow viscous liquid which was directly used in the next step without any purification.

¹H NMR (300 MHz, CDCl₃) δ 8.67 (s, 2H), 7.53 (s, 2H), 3.04 (d, 4H, J=6.61 Hz), 1.90 (m, 2H), 1.40-1.20 (m, 48H), 0.83-0.88 (m, 12H), 0.55 (s, 18H). ¹³C NMR (400 MHz, CDCl₃) 144.67, 137.52, 137.42, 133.89, 133.61, 133.54, 132.69, 120.99, 120.26, 53.37, 39.94, 35.09, 33.43, 31.93, 30.02, 29.68, 29.36, 26.72, 26.68, 22.68, 14.08, 8.07.

Synthesis of compound 6 Compound 4 (0.8 g, 1.0 mmol) was dissolved in dry THF (30 mL) under argon at room temperature. 2.5M of n-BuLi in hexane (0.84 mL, 2.1 mmol) was added dropwise. After stirring at room temperature for 20 minutes, 3 g of iodine[3 mmol) dissolved in 10 mL of anhydrous THF was transferred into the reaction mixture. The reaction was then quenched by 20 mL of water ten minutes later. 20 mL of 5% sodium hydroxide solution was added into the mixture and kept stirring for about 10 minutes. The mixture was then extracted with ethyl ether. The organic layer was washed with water several times and dried over anhydrous magnesium sulfate. After removal of the solvent, the residue was purified by flash chromatography on silica gel (hexane as eluent) to afford the 0.63 g of pure product (yield 60%) as a pale yellow liquid. ¹H NMR (300 MHz, CDCl₃) δ 8.54 (s, 2H), 7.43 (s, 2H) 3.00 (d, 4H, J=6.69 Hz), 1.90 (m, 2H), 1.40-1.20 (m, 48H), 0.88-0.83 (m, 12H). ¹³C NMR (400 MHz, CDCl₃) 144.76, 135.20, 134.88, 133.44, 132.94, 131.45, 120.07, 119.02, 74.18, 40.18, 35.44, 33.50, 32.00, 31.96, 30.18, 29.84, 29.75, 29.41, 26.77, 26.71, 22.75, 22.71, 14.16, 14.16.

Synthesis of homopolymer HMPQTN via Stilly coupling polymerization. To a 25 mL of round bottom flask equipped with a condenser was added 3 (450.8 mg, 0.4 mmol), 4 (421.2 mg, 0.4 mmol) and 20 mL of anhydrous toluene. The mixture was then evacuated and refilled with argon over three cycles to remove oxygen and finally was added Pd(PPh₃)₄ (23 mg, 0.02 mmol, 5% equiv.) under argon stream. The mixture was heated under reflux over 2 days. After cooling to room temperature, the organic solution was added dropwise to 100 mL of methanol to obtain precipitate, which was collected by filtration and washed with methanol and dried. The crude polymer was then extracted subsequently with methanol, acetone, hexane, and chloroform in a Soxhlet's extractor. The fraction from chloroform was concentrated under reduced pressure, and the residue was added dropwise to excess methanol to precipitate the polymer HMPQTN as a red solid (0.28 g, 44% yield).

Synthesis of D-A copolymer PQTN-BT. The polymerization was carried out in a scale of 0.5 mmol for each monomer using the same procedure as the preparation of HMPQTN. Subsequent Soxhlet extraction with methanol, acetone, hexane, and chloroform finally afforded 0.28 g of the polymer from chloroform fraction with the yield of 60%.

The foregoing is illustrative of the present invention, and is not to be construed as limiting thereof. The invention is defined by the following claims, with equivalents of the claims to be included therein.

That which is claimed is:

1. A copolymer comprising at least one donor monomer and at least one acceptor monomer,
said donor monomer selected from the group consisting of: 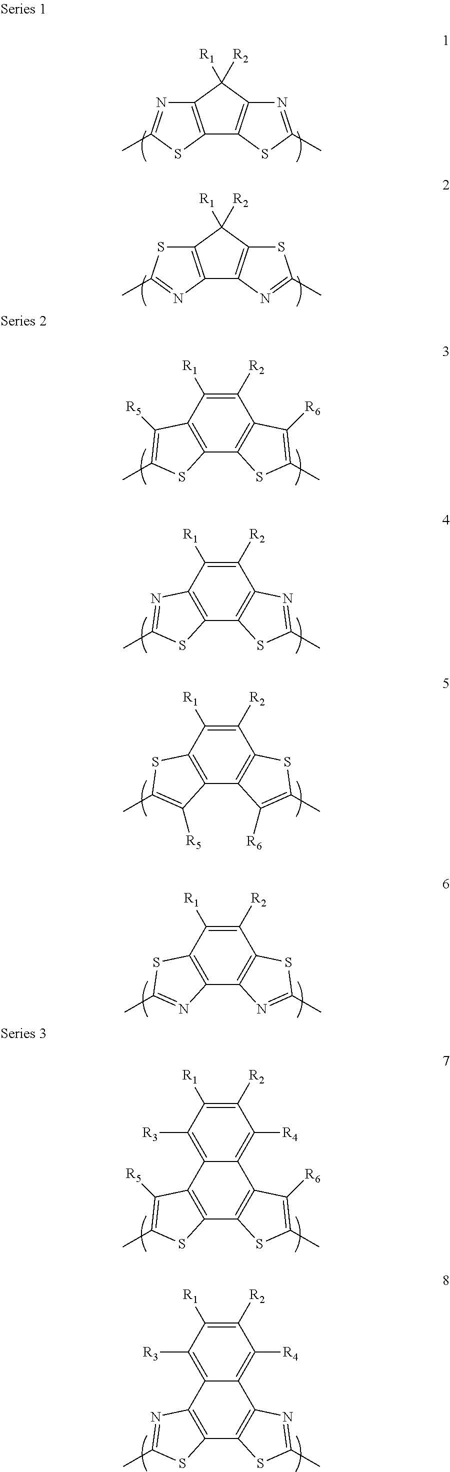 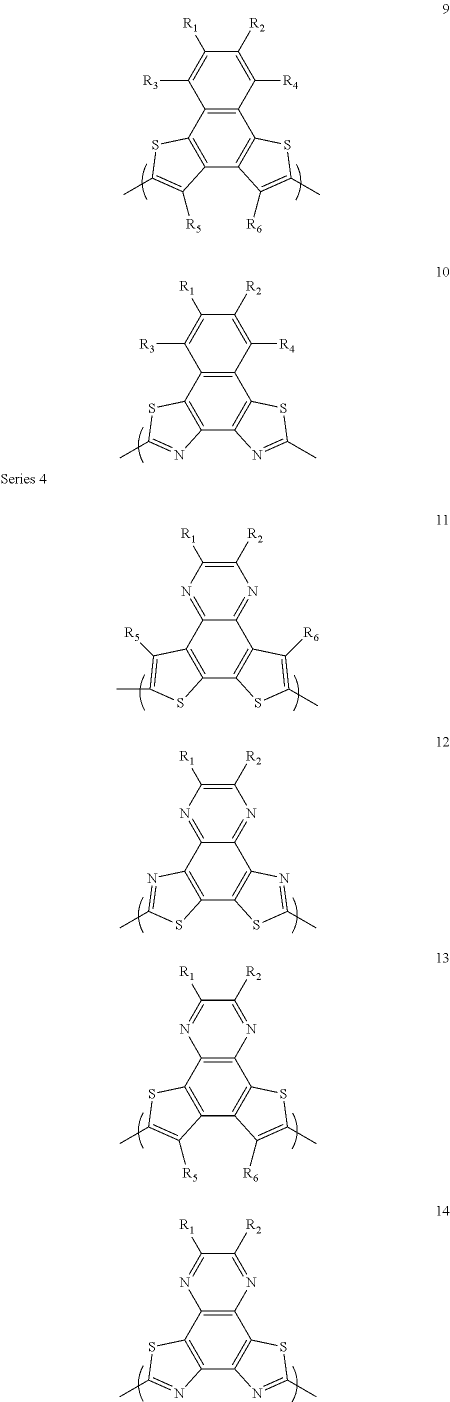 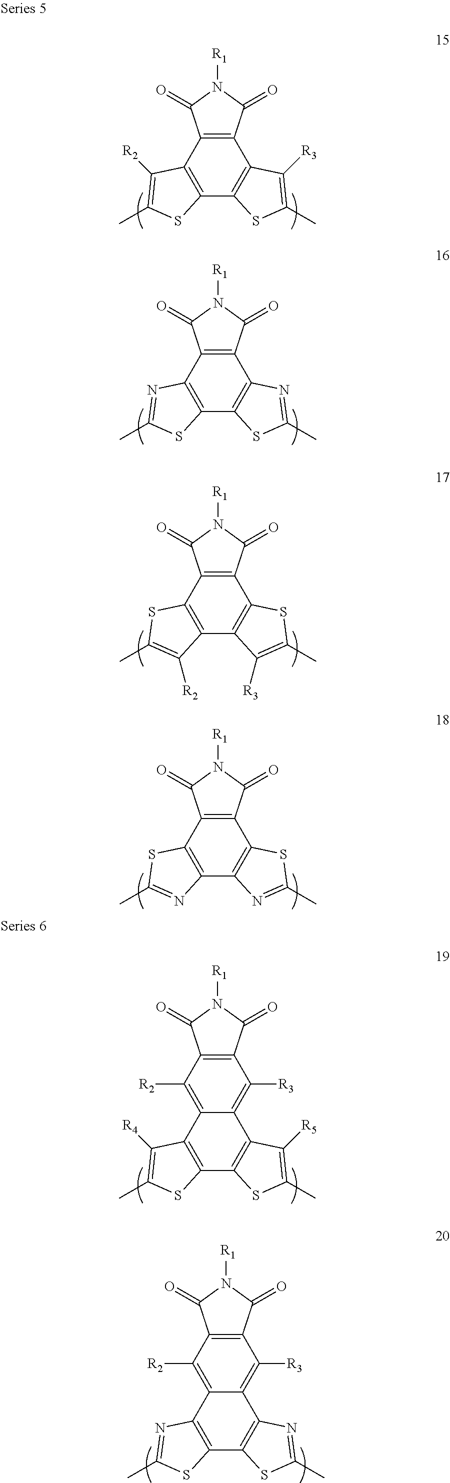 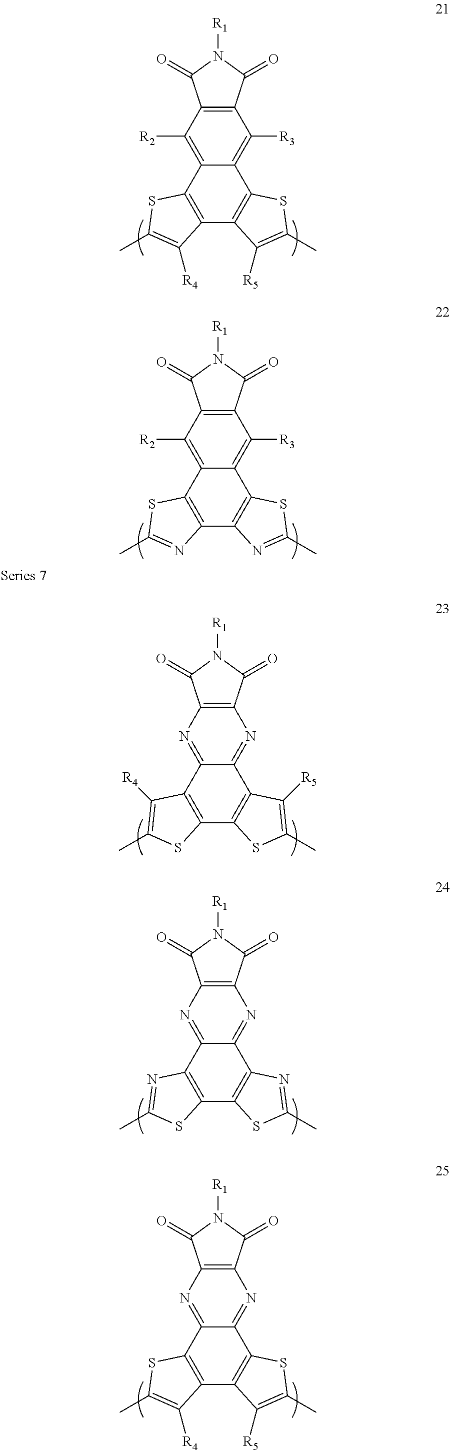 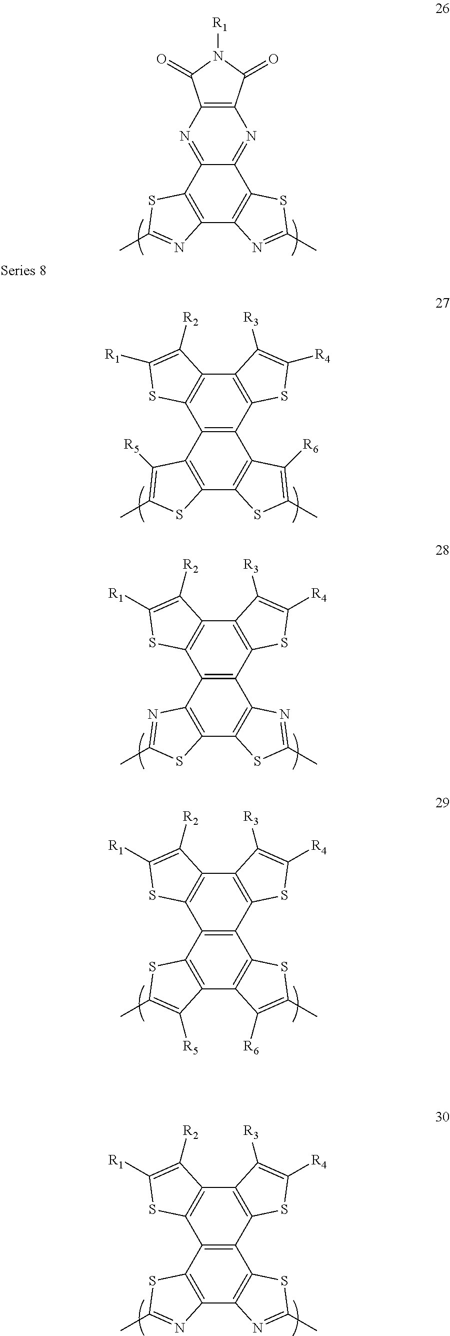 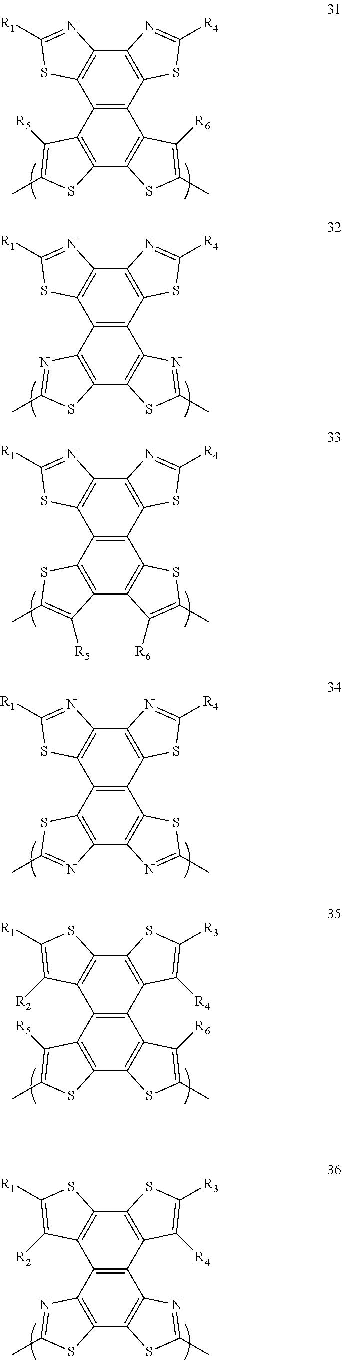 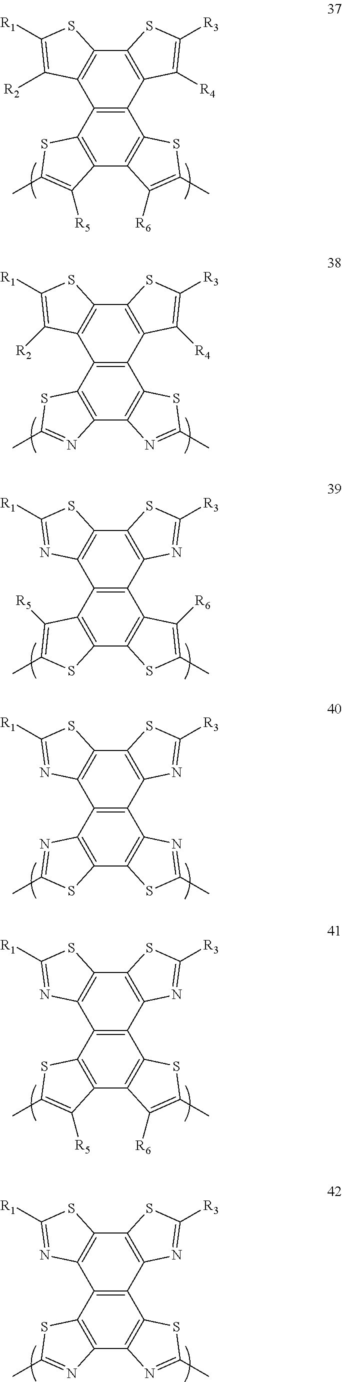 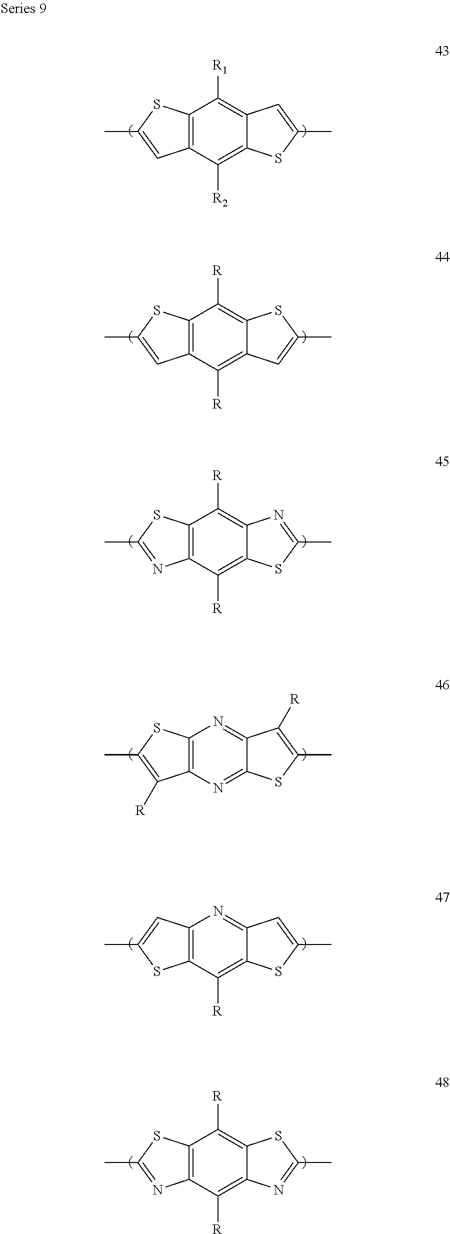
said acceptor monomer selected from the group consisting of: 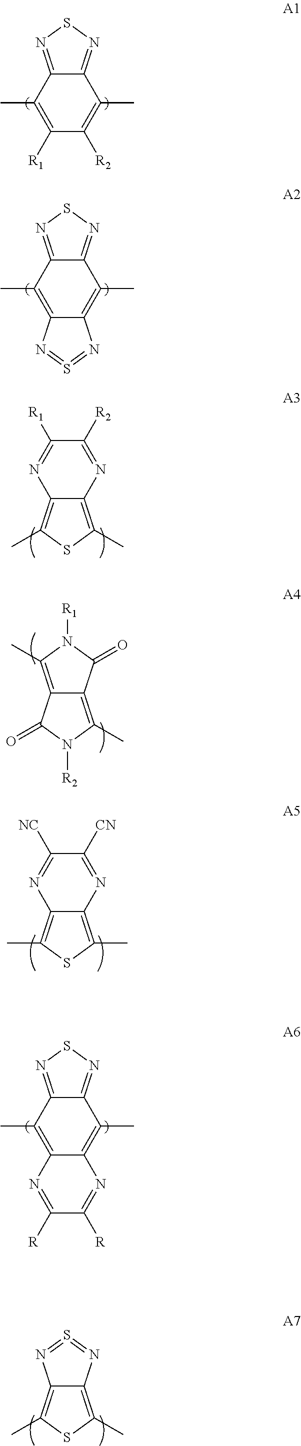 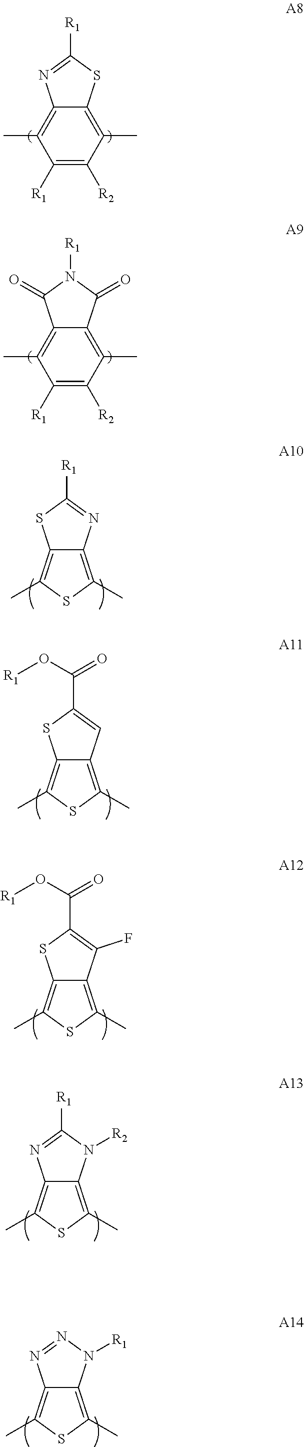 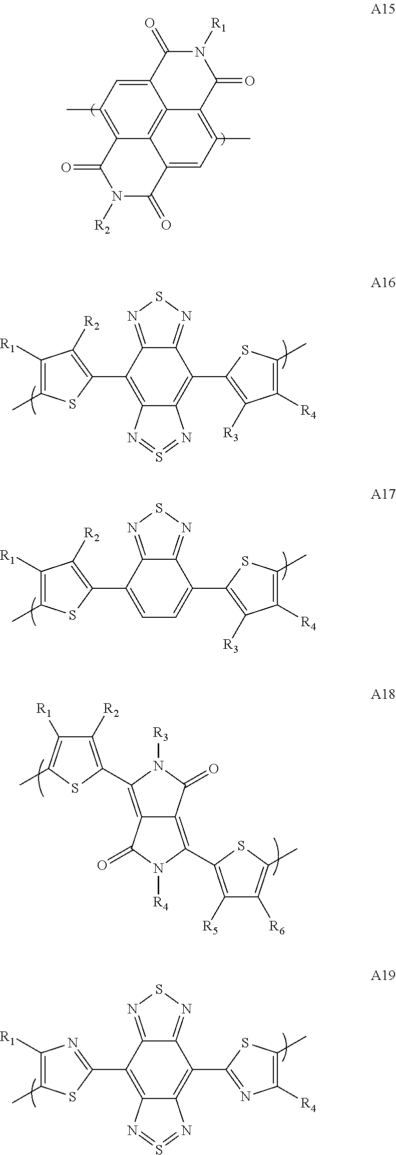 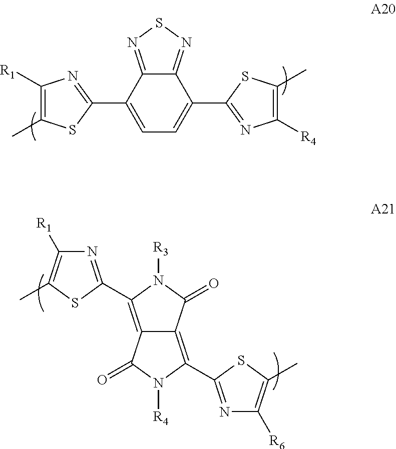
wherein each R₁, R₂, R₃, R₄, R₅, and R₆ is independently selected from the group consisting of H, C1-C20 alkyl, C1-C20 fluoroalkyl, C1-C20 alkoxy, C1-C20 fluoroalkoxy, halo, and aryl;
subject to the proviso that said acceptor monomer is not A1, A3, A11 or A12 when said donor monomer is 43.

2. The polymer of claim 1, subject to the proviso that said acceptor monomer is not A1, A2, A3, A10, A11, A12, A13 or A14 when said donor monomer is 43, 44, 46 or 47.

3. The polymer of claim 1 having a number average molecular weight of from 500 to 1,000,000 grams per mole.

4. The polymer of claim 1, further comprising at least one additional comonomers.

5. The polymer of claim 4, wherein said at least one additional comonomer is selected from the group consisting of: 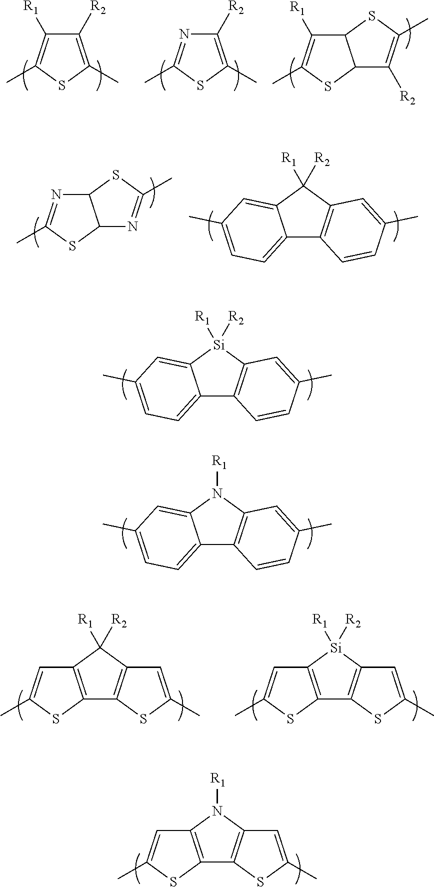
or combinations thereof, wherein R₁ and R₂ are as given above.

6. The polymer of claim 1, wherein said donor monomer is selected from the group consisting of:

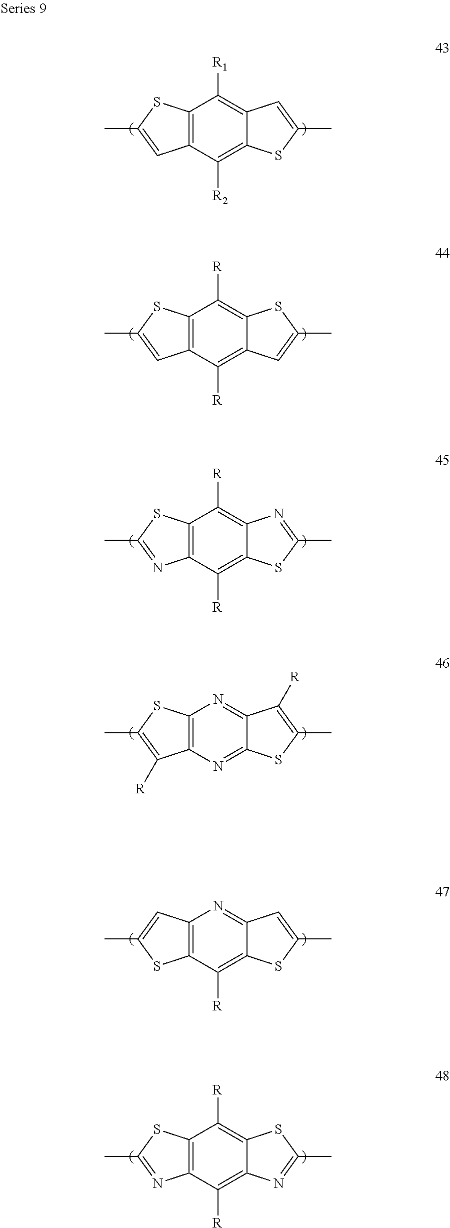

7. The polymer of claim 1, wherein said acceptor monomer is selected from the group consisting of:

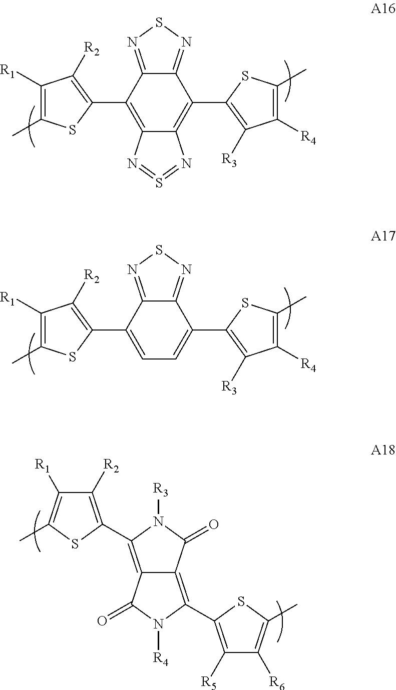

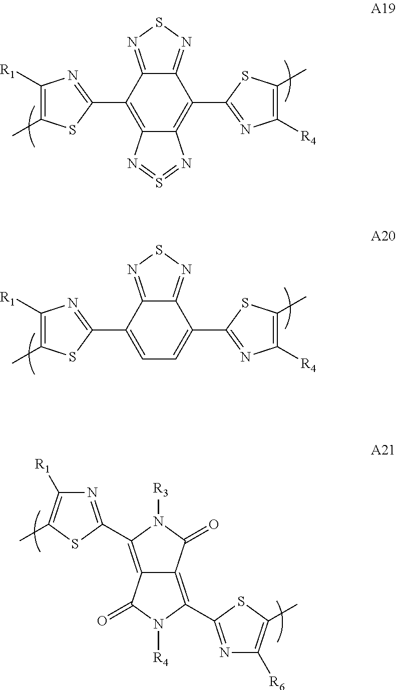

8. The polymer of claim 1 having the formula: 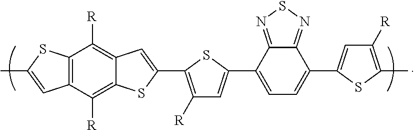
wherein each R is independently as given above.

9. The polymer of claim 1 having the formula: 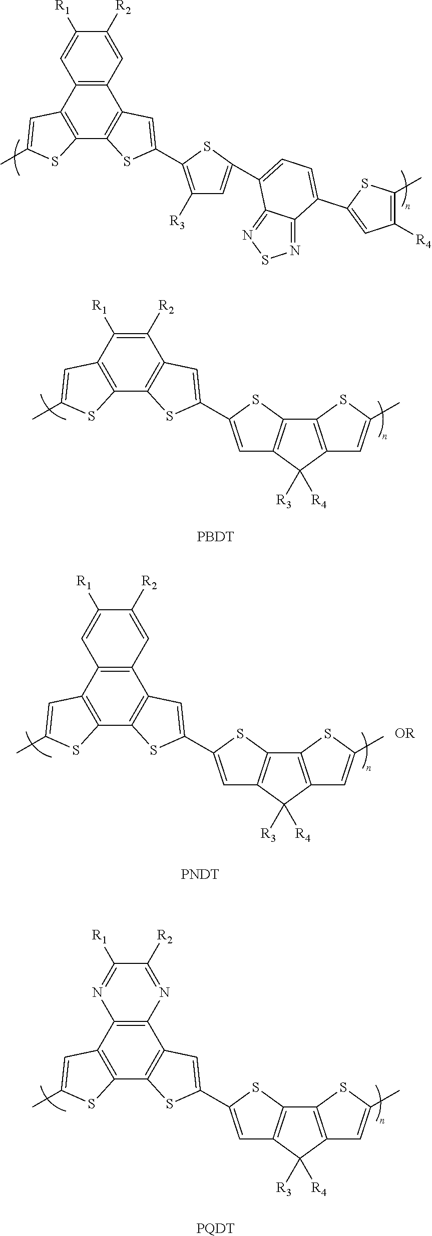
wherein each R₁, R₂, R₃ and R₄ is independently as given above.

10. A polymer comprising at least one donor monomer selected from the group consisting of: 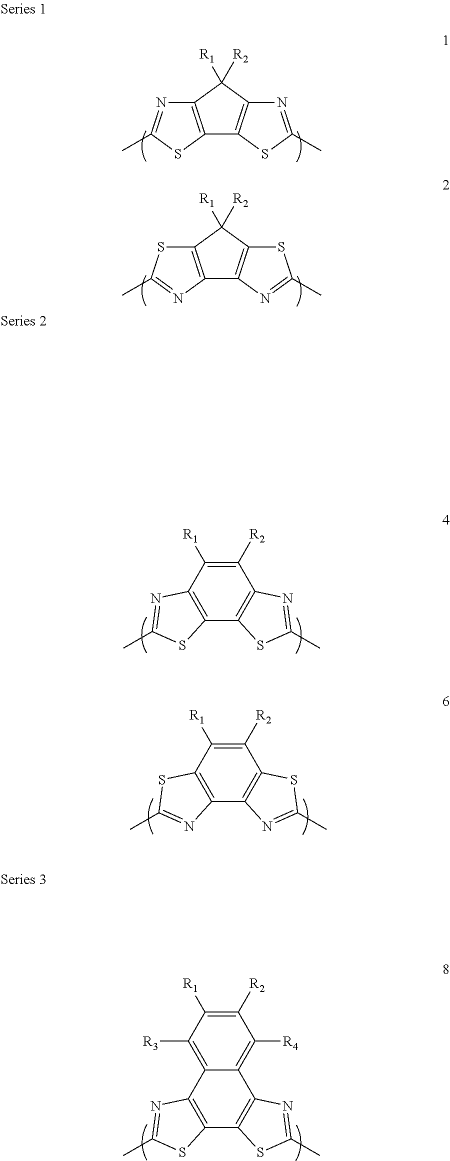 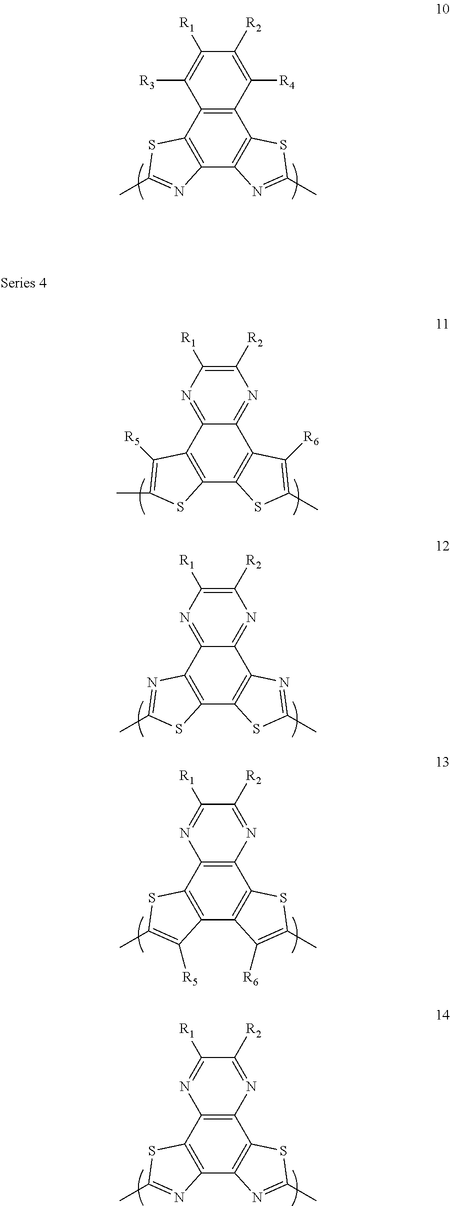 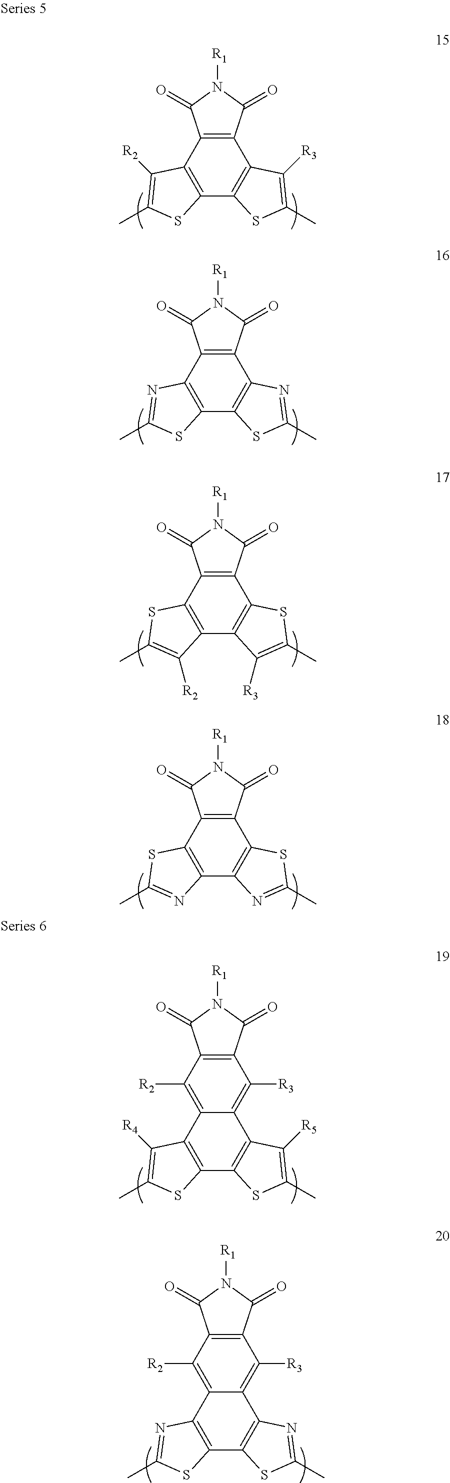 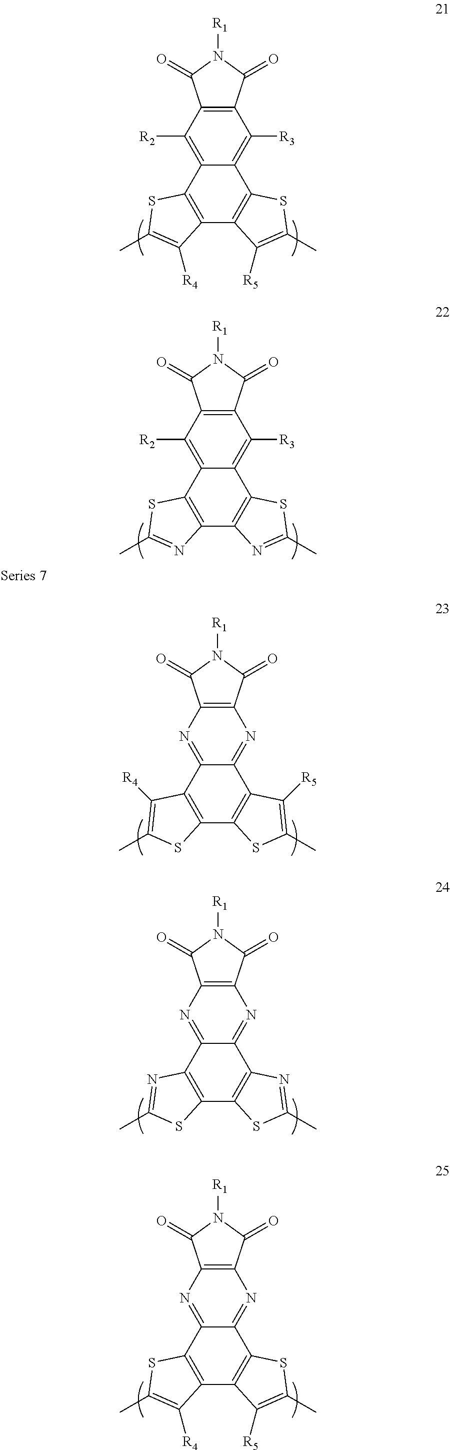 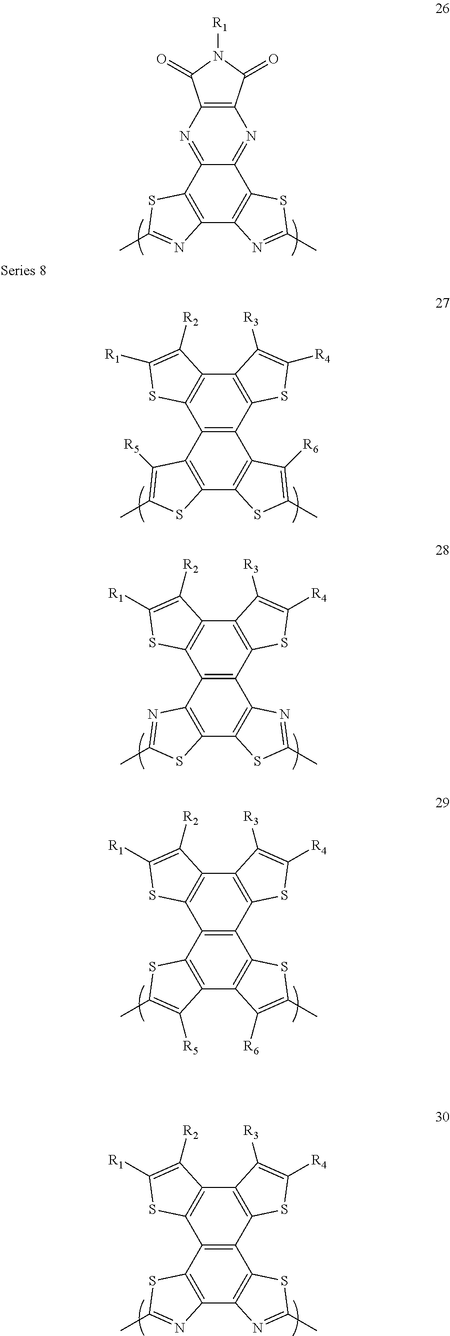 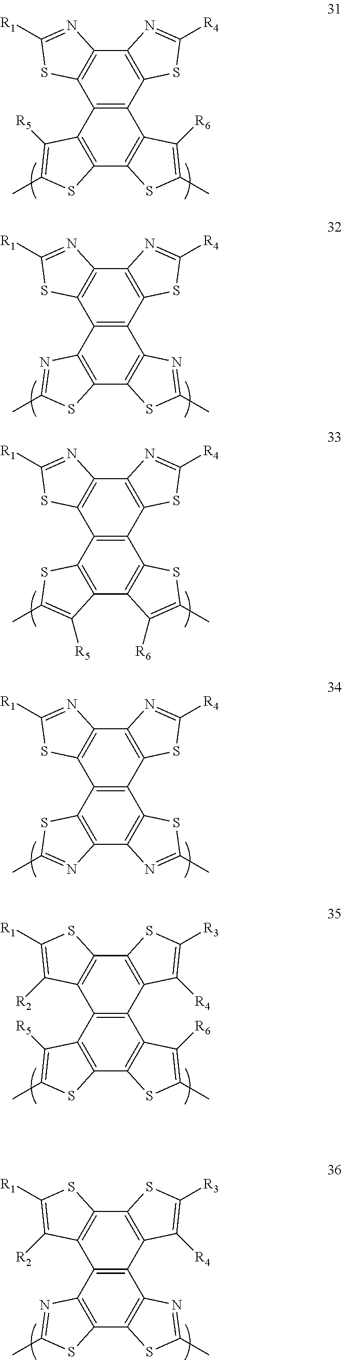 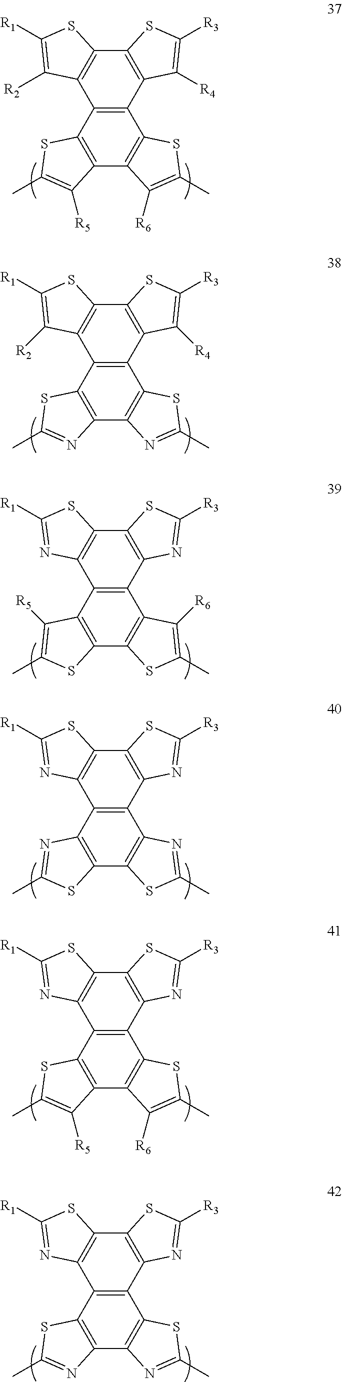 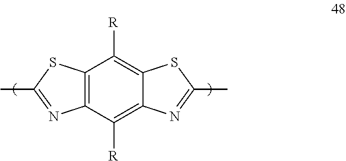
wherein each R₁, R₂, R₃, R₄, R₅, and R₆ is independently selected from the group consisting of H, C1-C20 alkyl, C1-C20 fluoroalkyl, C1-C20 alkoxy, C1-C20 fluoroalkoxy, halo, and aryl.

11. The polymer of claim 10 having a number average molecular weight of from 500 to 1,000,000 grams per mole.

12. The polymer of claim 10, wherein said polymer is a homopolymer.

13. The polymer of claim 10, wherein said polymer is a copolymer with at least one additional monomer.

14. The polymer of claim 13, wherein said copolymer comprises an acceptor monomer selected from the group consisting of:

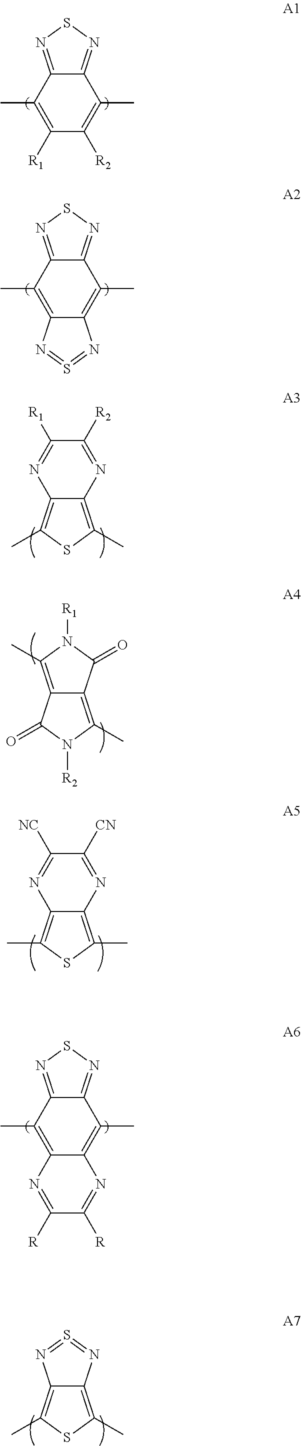

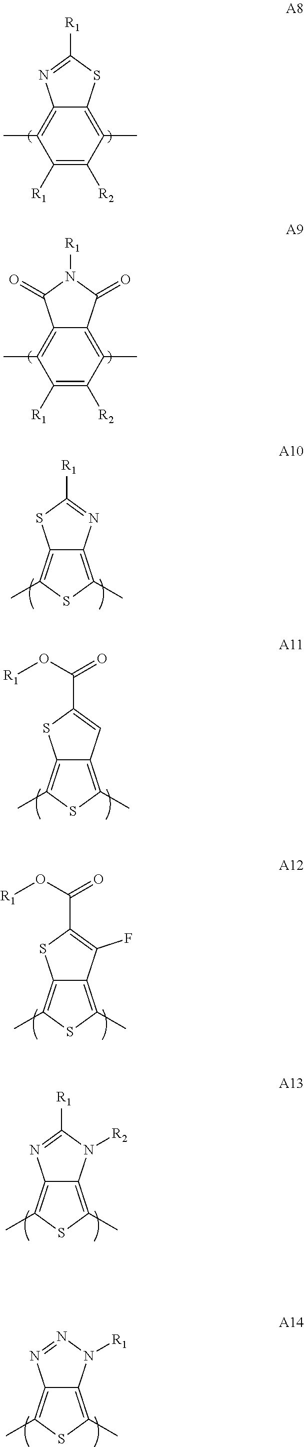

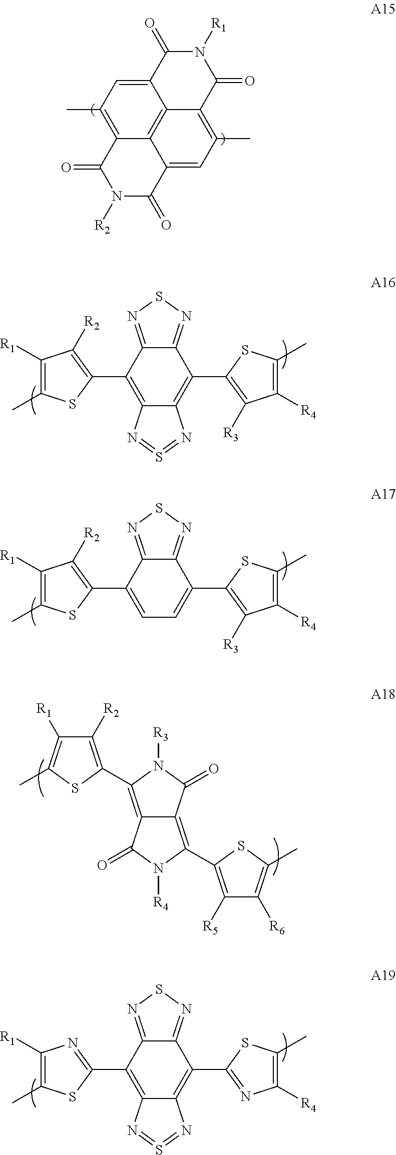

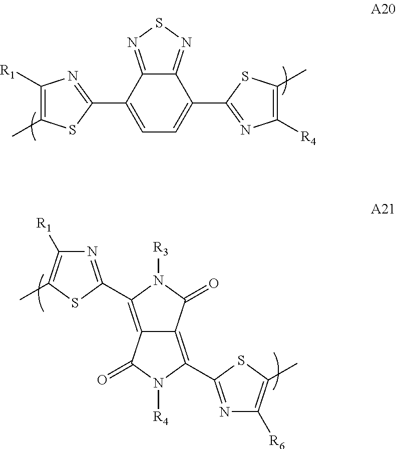

15. The copolymer of claim 13, wherein at least one of said additional comonomer is selected from the group consisting of: 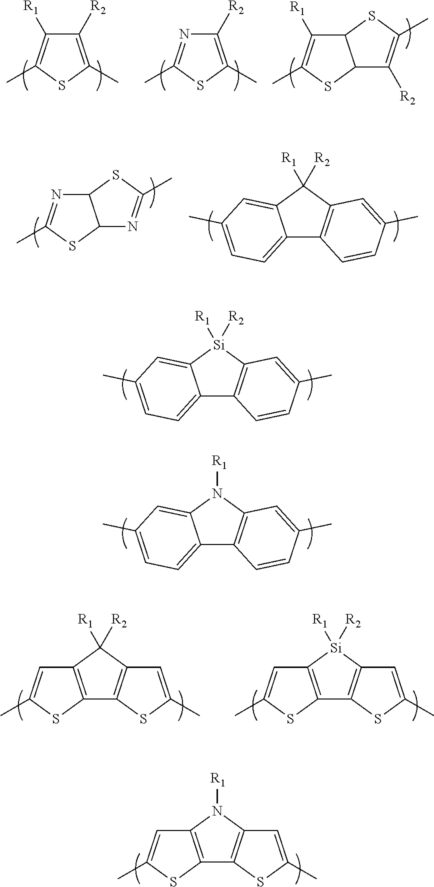
or combinations thereof, wherein R₁ and R₂ are as given above.

16. The polymer of claim 10, wherein said donor monomer is selected from the group consisting of:

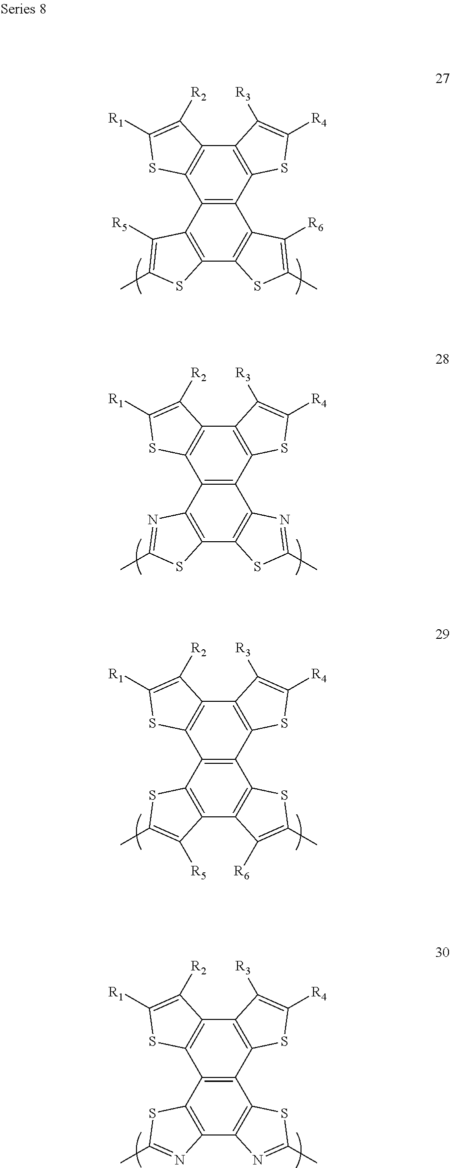

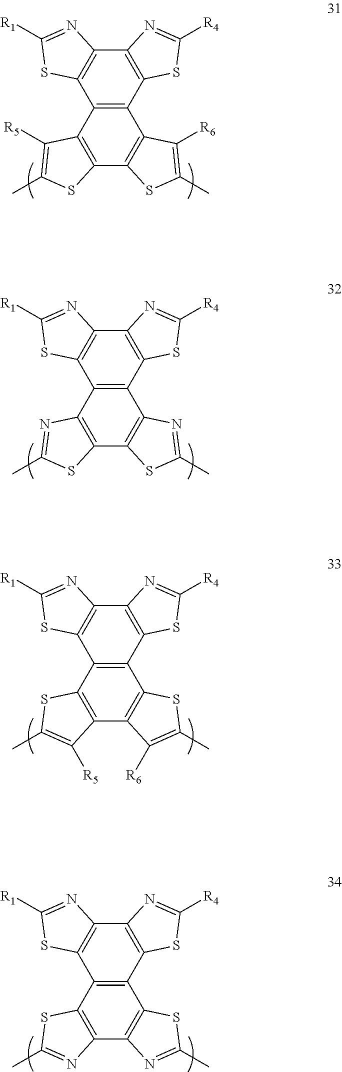

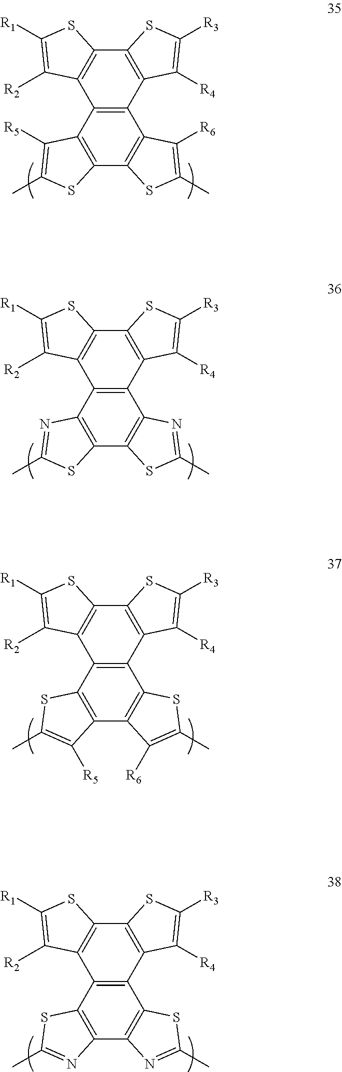

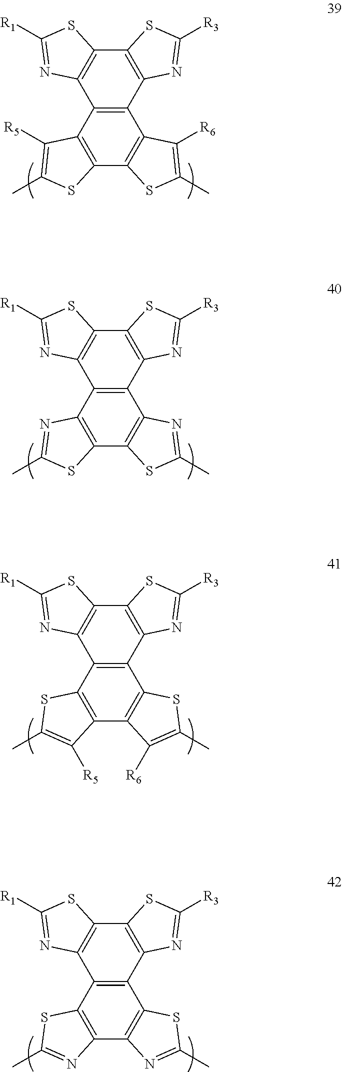

17. The polymer of claim 10, wherein said donor monomer is selected from the group consisting of:

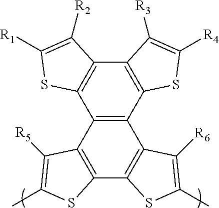

18. The polymer of claim 17 having the formula:

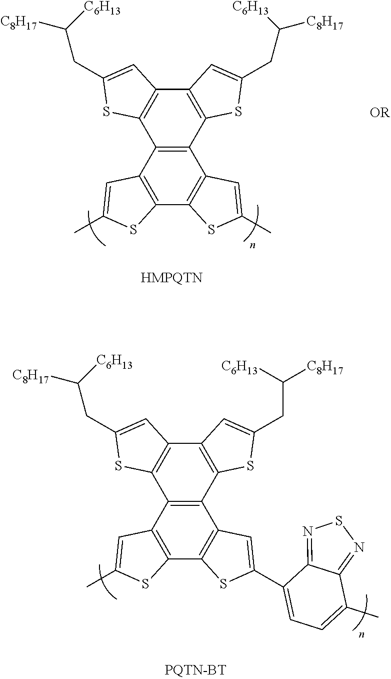

19. The polymer of claim 10, wherein said donor monomer is selected from the group consisting of:

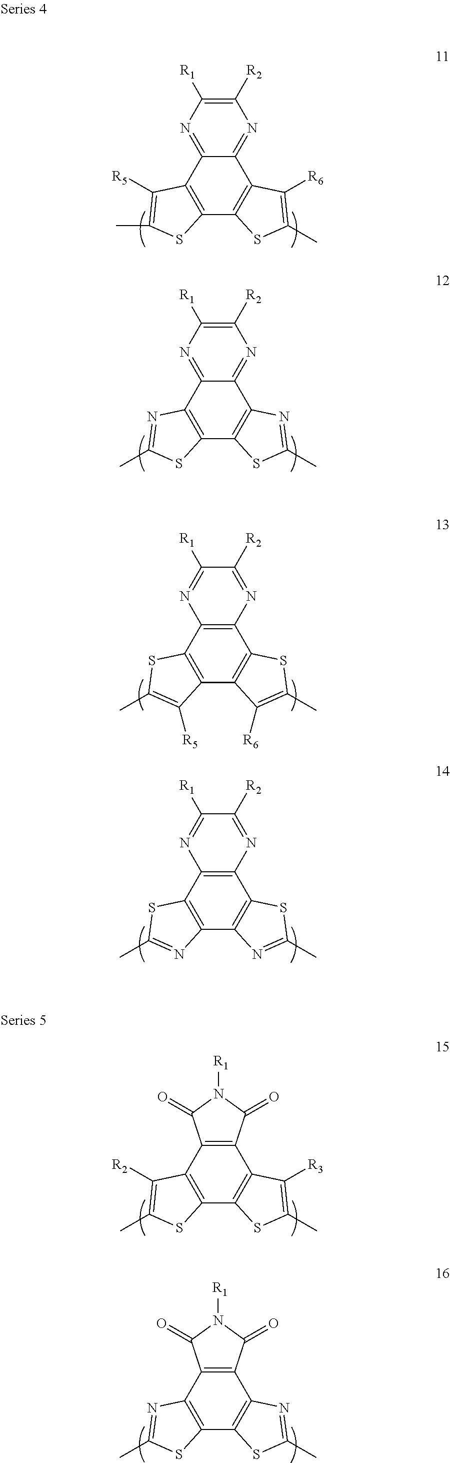

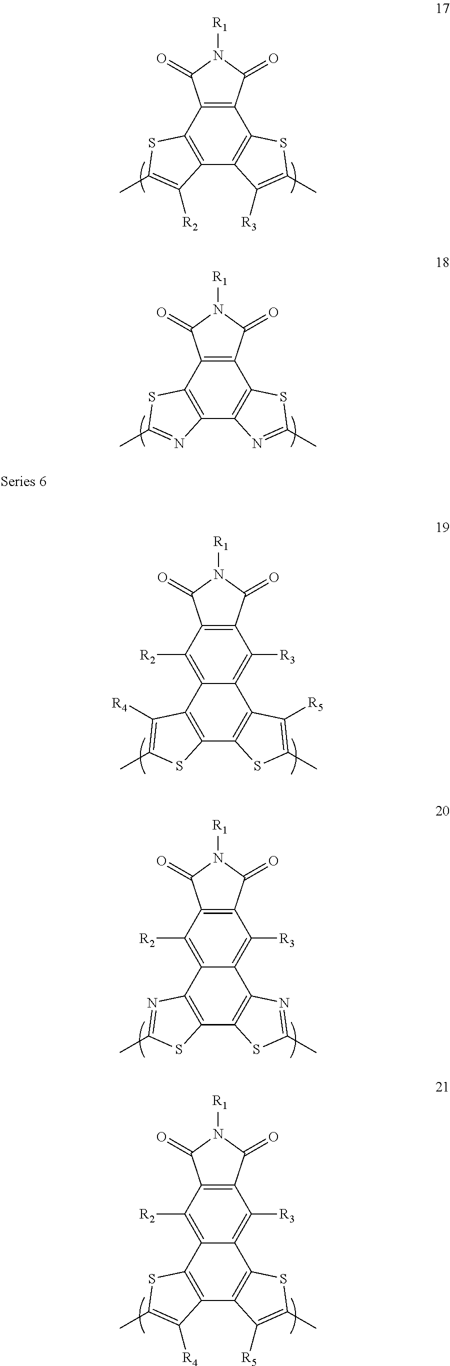

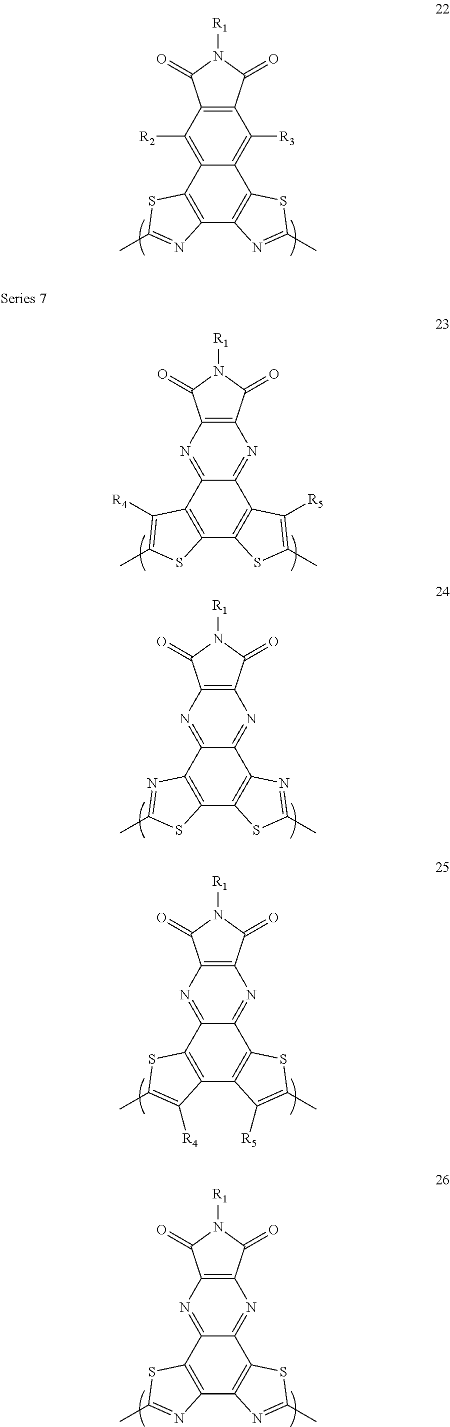

20. Use of a polymer of claim 1 as a charge-transport, semiconducting, el conducting, photoconducting, or light emitting material.

21. A microelectronic device comprising a polymer of claim 1.

22. The device of claim 21, wherein said device is an optoelectronic device.

23. The device of claim 21, wherein said polymer comprises a heterojunction in said device.

24. The device of claim 21, wherein said device is a photovoltaic cell, field effect transistor, light emitting diode, photodectector, photovoltaic detector, imaging device, lasing device, storage element, amplifier, emitter, or electrochromic display.

25. The device of claim 21, wherein said device comprises a first electrode, a second electrode, and a photoactive material disposed between said first and second electrode, said photoactive material comprising said polymer.

26. A monomer useful for the production of a polymer, said monomer selected from the group consisting of: 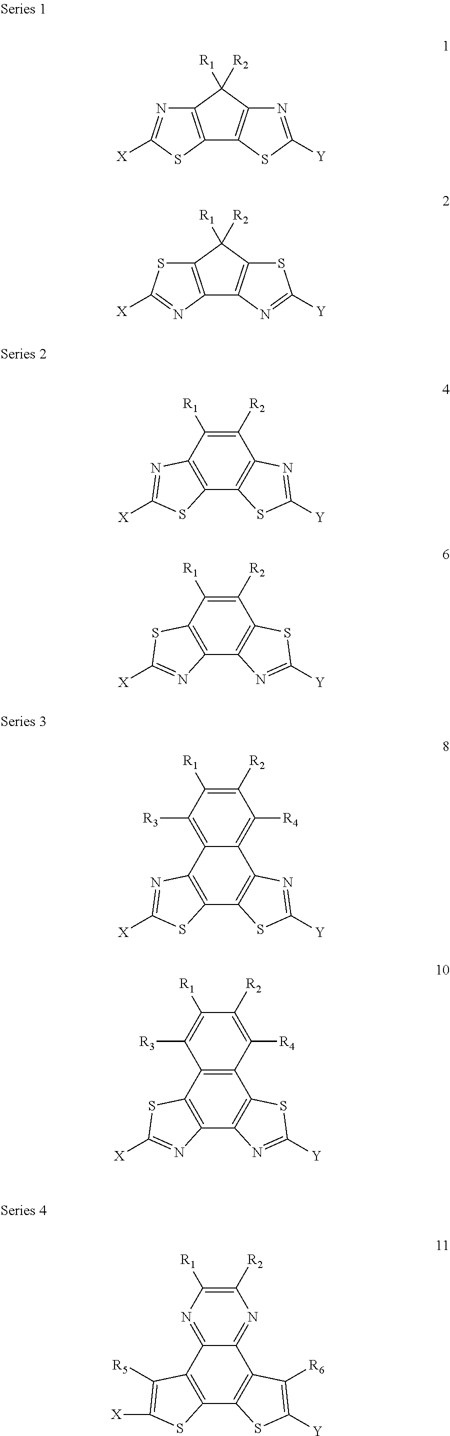 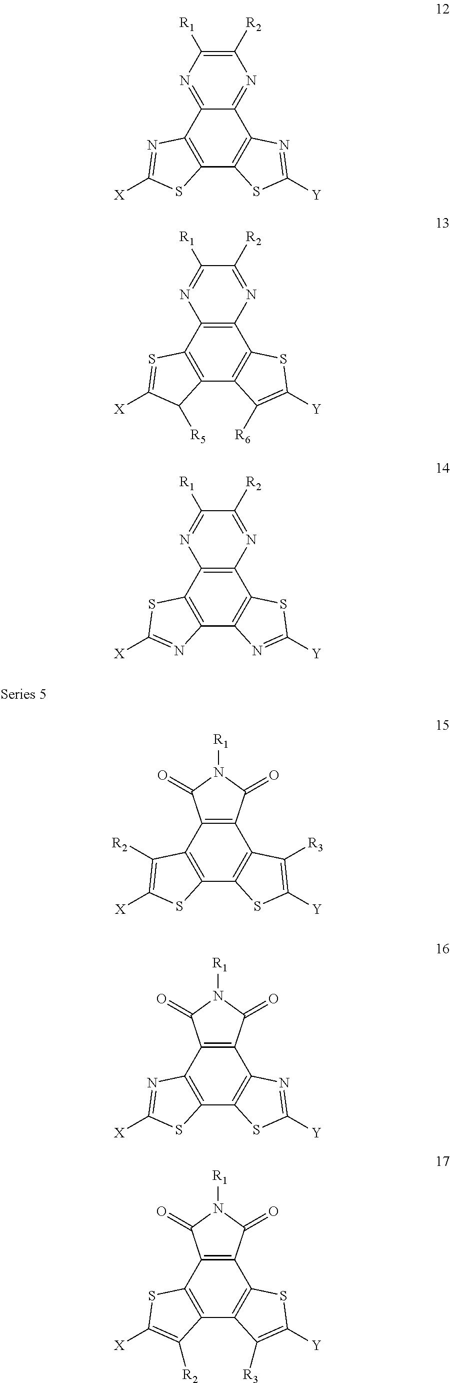 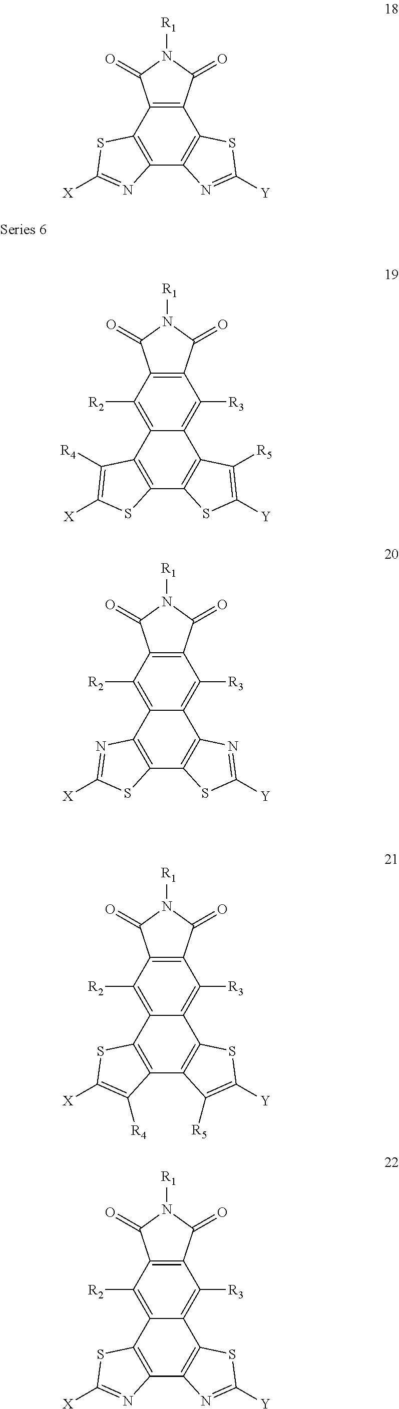 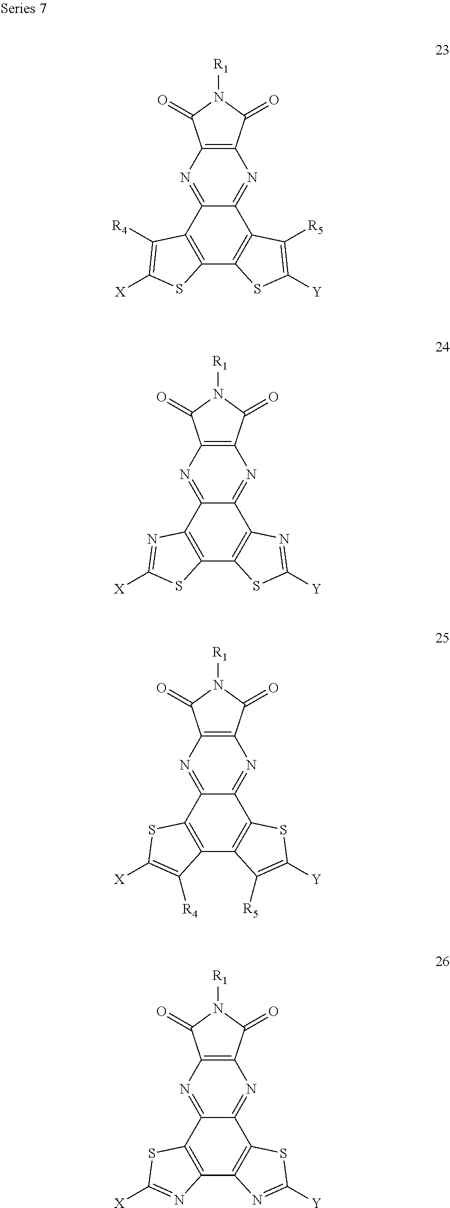 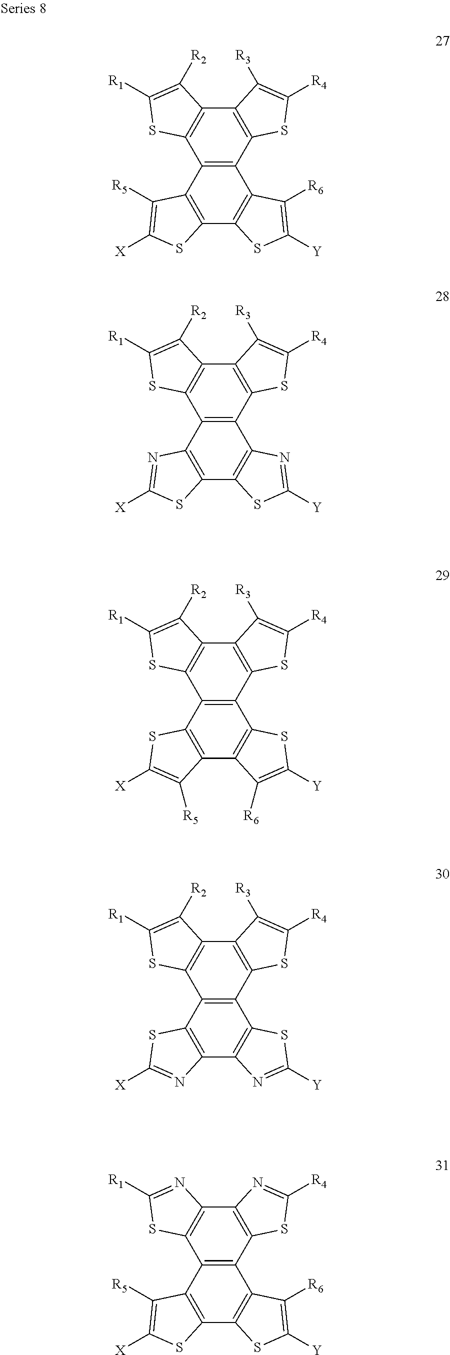 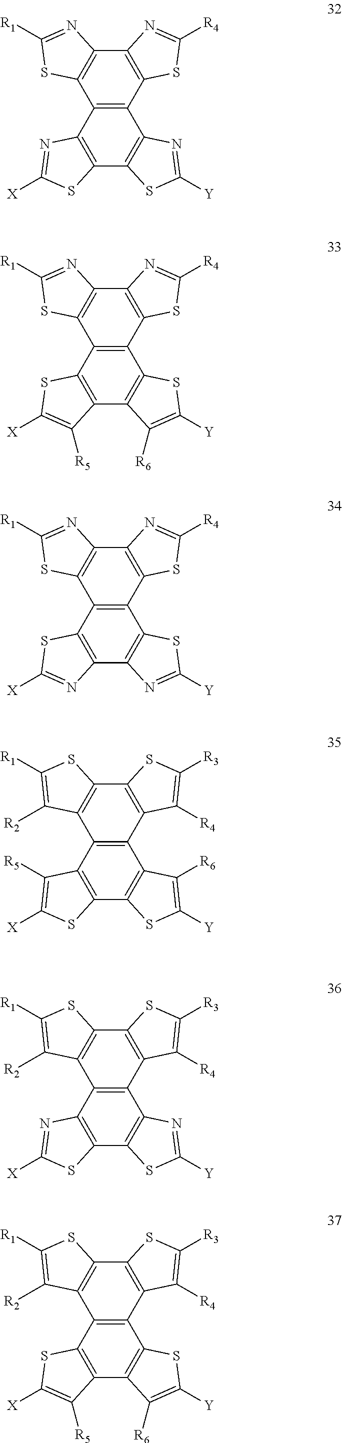 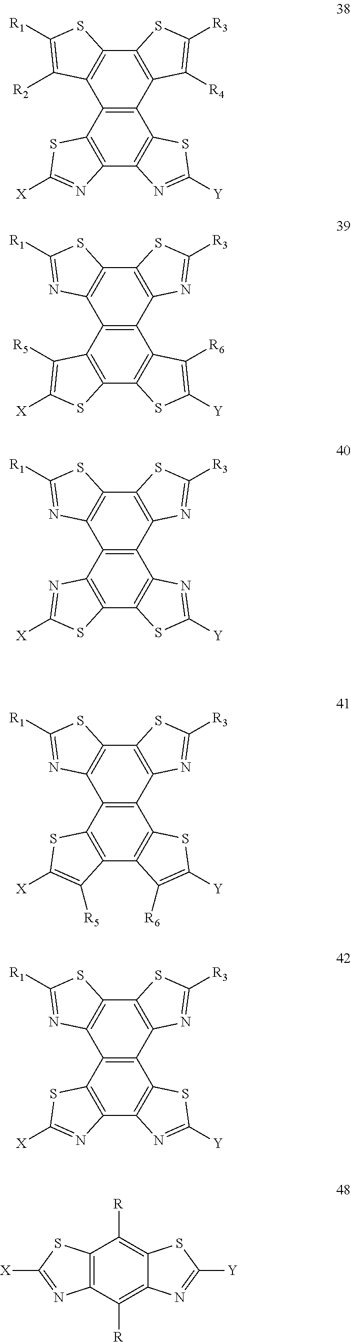
wherein each R₁, R₂, R₃, R₄, R₅, and R₆ is independently selected from the group consisting of H, C1-C20 alkyl, C1-C20 fluoroalkyl, C1-C20 alkoxy, C1-C20 fluoroalkoxy, halo, and aryl; and
X and Y are each independently selected from the group consisting of H and reactive functional groups.

27. The monomer of claim 26, wherein each of said reactive group is independently selected from reactive halide functional groups, reactive boron functional groups, and reactive tin functional groups.

28. The monomer of claim 26, wherein at least one of X and Y is a halide functional group.

29. The monomer of claim 26, wherein at least one of X and Y is either a boron functional group or a reactive tin functional group.

30. The monomer of claim 26, wherein one of X and Y is a halide functional group, and the other is either a boron functional group or a reactive tin functional group.

31. The monomer of claim 26, wherein both X and Y are halo.

32. The monomer of claim 26, where both X and Y are a trialkyltin.
